SEQUENCE LISTING (137 sequences)

SEQ ID NO: 1 - protein, 6 aa, synthetic construct
DRVSQS

SEQ ID NO: 2 - protein, 6 aa, synthetic construct
IYSNGD

SEQ ID NO: 3 - protein, 15 aa, synthetic construct
CAVRDSGAGSYQLTF

SEQ ID NO: 4 - protein, 5 aa, synthetic construct
SGDLS

SEQ ID NO: 5 - protein, 6 aa, synthetic construct
YYNGEE

SEQ ID NO: 6 - protein, 14 aa, synthetic construct
CASSVDSSHNEQFF

SEQ ID NO: 7 - DNA, 402 bp, synthetic construct
  source 1..402: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgagtttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagggactctggggctgggagttaccaactcactttcgggaaggggaccaaactctcggtcatacca

SEQ ID NO: 8 - DNA, 402 bp, synthetic construct
  source 1..402: mol_type = other DNA
atgaagtctctgagggtgctgctggtcatcctgtggctgcagctgtcttgggtgtggagccagcagaaggaggtggagcagaactccggaccactgtctgtgcctgagggagccatcgccagcctgaattgcacatactccgacagagtgagccagtccttcttttggtacaggcagtattccggcaagtctcccgagctgatcatgagcatctactccaacggcgacaaggaggatggcagattcaccgcccagctgaataaggccagccagtacgtgagcctgctgatccgcgactcccagccctctgatagcgccacatacctgtgcgccgtgcgggattctggagcaggcagctatcagctgacctttggcaagggcacaaagctgagcgtgatccct

SEQ ID NO: 9 - protein, 134 aa, synthetic construct
MKSLRVLLVILWLQLSWVWSQQKEVEQNSGPLSVPEGAIASLNCTYSDRVSQSFFWYRQYSGKSPELIMSIYSNGDKEDGRFTAQLNKASQYVSLLIRDSQPSDSATYLCAVRDSGAGSYQLTFGKGTKLSVIP

SEQ ID NO: 10 - DNA, 396 bp, synthetic construct
  source 1..396: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagattcctcccacaatgagcagttcttcgggccagggacacggctcaccgtgcta

SEQ ID NO: 11 - DNA, 396 bp, synthetic construct
  source 1..396: mol_type = other DNA
atgggcttccggctgctgtgctgcgtggcattttgcctgctgggagcaggaccagtggactccggcgtgacccagacacccaagcacctgatcaccgcaacaggacagagggtgaccctgagatgttcccctaggtctggcgacctgagcgtgtactggtatcagcagagcctggatcagggcctgcagttcctgatccagtactataacggcgaggagagagccaagggcaatatcctggagaggttcagcgcccagcagtttccagacctgcactccgagctgaatctgagctccctggagctgggcgacagcgccctgtacttctgcgcctctagcgtggattcctctcacaacgagcagttctttggaccaggaaccaggctgacagtgctg

SEQ ID NO: 12 - protein, 132 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDSSHNEQFFGPGTRLTVL

SEQ ID NO: 13 - protein, 274 aa, synthetic construct
MKSLRVLLVILWLQLSWVWSQQKEVEQNSGPLSVPEGAIASLNCTYSDRVSQSFFWYRQYSGKSPELIMSIYSNGDKEDGRFTAQLNKASQYVSLLIRDSQPSDSATYLCAVRDSGAGSYQLTFGKGTKLSVIPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDSSHNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 15 - protein, 5 aa, synthetic construct
SIFNT

SEQ ID NO: 16 - protein, 7 aa, synthetic construct
LYKAGEL

SEQ ID NO: 17 - protein, 13 aa, synthetic construct
CAGQLEGDDKIIF

SEQ ID NO: 18 - protein, 5 aa, synthetic construct
KGHSH

SEQ ID NO: 19 - protein, 6 aa, synthetic construct
LQKENI

SEQ ID NO: 20 - protein, 13 aa, synthetic construct
CASSRSWPPGYTF

SEQ ID NO: 21 - DNA, 387 bp, synthetic construct
  source 1..387: mol_type = other DNA
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcagctagagggggatgacaagatcatctttggaaaagggacacgacttcatattctcccc

SEQ ID NO: 22 - DNA, 387 bp, synthetic construct
  source 1..387: mol_type = other DNA
atgctccttgaacatttattaataatcttgtggatgcagctgacatgggtcagtggtcaacagctgaatcagagtcctcaatctatgtttatacaagagggagaagatgtctccatgaactgcacttcttcaagcatatttaacacctggctatggtacaagcaggaccctggggaaggtcctgtcctcttgattgccttatataaggctggtgaattgacctcaaatggaagactgactgctcagtttggtataaccagaaaggacagcttcctgaatatctcagcatccatacctagtgatgtaggcatctacttctgtgctggtcaactagagggggatgacaagatcatctttggaaaagggacacgacttcatattctcccc

SEQ ID NO: 23 - protein, 129 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQLEGDDKIIFGKGTRLHILP

SEQ ID NO: 24 - DNA, 396 bp, synthetic construct
  source 1..396: mol_type = other DNA
atggacaccagactactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcacgctcttggccccctggctacaccttcggttcggggaccaggttaaccgttgta

SEQ ID NO: 25 - DNA, 396 bp, synthetic construct
  source 1..396: mol_type = other DNA
atggacaccagactactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtataggcaattaccagaggaaggtctgaaattcatggtttatttacaaaaggaaaatatcatagatgagtcaggaatgccaaaggaacgattttctgctgaatttcccaaagagggccccagcatcctgaggatccagcaagtcgtgcgaggagattcggcggcttatttctgtgcgtcttctcgctcttggccccctggctacaccttcggttcggggaccaggttaaccgttgta

SEQ ID NO: 26 - protein, 132 aa, synthetic construct
MDTRLLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSRSWPPGYTFGSGTRLTVV

SEQ ID NO: 27 - protein, 269 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQLEGDDKIIFGKGTRLHILPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 309 aa, synthetic construct
MDTRLLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSRSWPPGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 7 aa, synthetic construct
TSESDYY

SEQ ID NO: 30 - protein, 8 aa, synthetic construct
QEAYKQQN

SEQ ID NO: 31 - protein, 15 aa, synthetic construct
CAYWGWETSGSRLTF

SEQ ID NO: 32 - protein, 5 aa, synthetic construct
MNHEY

SEQ ID NO: 33 - protein, 6 aa, synthetic construct
SVGAGI

SEQ ID NO: 34 - protein, 12 aa, synthetic construct
CASRDMTGKAFF

SEQ ID NO: 35 - DNA, 408 bp, synthetic construct
  source 1..408: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttattggggttgggaaaccagtggctctaggttgacctttggggaaggaacacagctcacagtgaatcct

SEQ ID NO: 36 - DNA, 408 bp, synthetic construct
  source 1..408: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccaaaaggcagccaaatccttctcgctcaagatctcagactcacagctgggggatgccgcgatgtatttctgtgcttattggggttgggaaaccagtggctctaggttgacctttggggaaggaacacagctcacagtgaatcct

SEQ ID NO: 37 - protein, 136 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYWGWETSGSRLTFGEGTQLTVNP

SEQ ID NO: 38 - DNA, 390 bp, synthetic construct
  source 1..390: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagggatatgacagggaaggctttctttggacaaggcaccagactcacagttgta

SEQ ID NO: 39 - DNA, 390 bp, synthetic construct
  source 1..390: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgccggtatcactgaccaaggagaagtccccaatggctacaatgtctcgcgttctacgactgaagatttcccgctcaggctgctgtcggctgctcccagtcaaacgtctgtgtacttctgtgcgtctagagatatgacagggaaggctttctttggacaaggcaccagactcacagttgta

SEQ ID NO: 40 - protein, 130 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRDMTGKAFFGQGTRLTVV

SEQ ID NO: 41 - protein, 276 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYWGWETSGSRLTFGEGTQLTVNPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 307 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRDMTGKAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 9 aa, Homo sapiens
VVGAVGVGK

SEQ ID NO: 44 - protein, 10 aa, Homo sapiens
VVVGAVGVGK

SEQ ID NO: 45 - protein, 9 aa, Homo sapiens
AVGVGKSAL

SEQ ID NO: 46 - protein, 17 aa, synthetic construct
SARDRGLVSLPSVEAFF

SEQ ID NO: 47 - protein, 14 aa, synthetic construct
ASYLSGSIYNEQFF

SEQ ID NO: 48 - protein, 12 aa, synthetic construct
ASSYSTERGTIY

SEQ ID NO: 49 - protein, 11 aa, synthetic construct
ASSLADIYEQY

SEQ ID NO: 50 - protein, 12 aa, synthetic construct
CASSARNDEAFF

SEQ ID NO: 51 - protein, 12 aa, synthetic construct
CASSLGDSEQYF

SEQ ID NO: 52 - protein, 14 aa, synthetic construct
CASSQRSNTGELFF

SEQ ID NO: 53 - protein, 14 aa, synthetic construct
CASGGRDSTDTQYF

SEQ ID NO: 54 - protein, 14 aa, synthetic construct
ASSTSFWEVNTEAF

SEQ ID NO: 55 - protein, 12 aa, synthetic construct
ASSKRGWPYEQY

SEQ ID NO: 56 - protein, 11 aa, synthetic construct
ASSLADIYEQY

SEQ ID NO: 57 - protein, 11 aa, synthetic construct
ASSSTDRIEAF

SEQ ID NO: 58 - protein, 15 aa, synthetic construct
ASTTFKTGRAIEKLF

SEQ ID NO: 59 - protein, 13 aa, synthetic construct
ASSSRGHSGTEAF

SEQ ID NO: 60 - protein, 13 aa, synthetic construct
ASSSRGHSGTEAF

SEQ ID NO: 61 - protein, 10 aa, synthetic construct
ATYKVGDEQF

SEQ ID NO: 62 - protein, 14 aa, synthetic construct
ASSDWLAGAKDEQY

SEQ ID NO: 63 - protein, 11 aa, synthetic construct
ASSLVASNEQF

SEQ ID NO: 64 - protein, 13 aa, synthetic construct
ASSLGLLLYNEQF

SEQ ID NO: 65 - protein, 12 aa, synthetic construct
ASSLGDSYEQYF

SEQ ID NO: 66 - protein, 12 aa, synthetic construct
ASSEWGSTGELF

SEQ ID NO: 67 - protein, 12 aa, synthetic construct
ASSEWGSTGELF

SEQ ID NO: 68 - protein, 11 aa, synthetic construct
ASSEYTMGTQY

SEQ ID NO: 69 - protein, 271 aa, synthetic construct
MKSLRVLLVILWLQLSWVWSQQKEVEQNSGPLSVPEGAIASLNCTYSDRVSQSFFWYRQYSGKSPELIMSIYSNGDKEDGRFTAQLNKASQYVSLLIRDSQPSDSATYLCAVRDSGAGSYQLTFGKGTKLSVIPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 70 - protein, 305 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDSSHNEQFFGPGTRLTVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 71 - protein, 267 aa, synthetic construct
METLLGVSLVILWLQLARVNGQQLNQSPQSMFIQEGEDVSMNCTSSSIFNTWLWYKQDPGEGPVLLIALYKAGELTSNGRLTAQFGITRKDSFLNISASIPSDVGIYFCAGQLEGDDKIIFGKGTRLHILPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 72 - protein, 305 aa, synthetic construct
MGTRLLCWAALCLLGAELTNAGVMQNPRHLVRRRGQEARLRCSPMKGHSHVYWYRQLPEEGLKFMVYLQKENIIDESGMPKERFSAEFPKEGPSILRIQQVVRGDSAAYFCASSRSWPPGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 73 - protein, 272 aa, synthetic construct
METLLGVSLVILWLQLARVNAQTVTQSQPEMSVQEAETVTLSCTYDTSESDYYLFWYKQPPSRQMILVIRQEAYKQQNATENRFSVNFQKAAKSFSLKISDSQLGDAAMYFCAYWGWETSGSRLTFGEGTQLTVNPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 74 - protein, 303 aa, synthetic construct
MGTRLLCWAALCLLGAELTNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRDMTGKAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 75 - DNA, 813 bp, synthetic construct
  source 1..813: mol_type = other DNA
atgaagtctctgagggtgctgctggtcatcctgtggctgcagctgtcttgggtgtggagccagcagaaggaggtggagcagaactccggaccactgtctgtgcctgagggagccatcgccagcctgaattgcacatactccgacagagtgagccagtccttcttttggtacaggcagtattccggcaagtctcccgagctgatcatgagcatctactccaacggcgacaaggaggatggcagattcaccgcccagctgaataaggccagccagtacgtgagcctgctgatccgcgactcccagccctctgatagcgccacatacctgtgcgccgtgcgggattctggagcaggcagctatcagctgacctttggcaagggcacaaagctgagcgtgatccctgatatccaaaaccctgagcccgccgtgtaccagctgaaagatcctagatcccaggactccacactgtgcctctttaccgatttcgacagccagatcaatgtgcccaagaccatggagagcggcaccttcatcaccgataagacagtgctcgatatgaaagccatggacagcaagtccaatggcgccattgcttggagcaaccagacctccttcacctgccaggacatttttaaagagaccaacgccacctaccccagcagcgacgtgccttgtgatgccaccctgaccgagaagagcttcgaaaccgacatgaacctgaactttcagaacctgagcgtcatgggactcagaatcctcctgctcaaagtcgccggcttcaacctcctgatgaccctgagactgtggagctcc

SEQ ID NO: 76 - DNA, 915 bp, synthetic construct
  source 1..915: mol_type = other DNA
atgggcttccggctgctgtgctgcgtggcattttgcctgctgggagcaggaccagtggactccggcgtgacccagacacccaagcacctgatcaccgcaacaggacagagggtgaccctgagatgttcccctaggtctggcgacctgagcgtgtactggtatcagcagagcctggatcagggcctgcagttcctgatccagtactataacggcgaggagagagccaagggcaatatcctggagaggttcagcgcccagcagtttccagacctgcactccgagctgaatctgagctccctggagctgggcgacagcgccctgtacttctgcgcctctagcgtggattcctctcacaacgagcagttctttggaccaggaaccaggctgacagtgctgaaggatctgcggaatgtgaccccccctaaggtgtccctgtttgagccctctaaggccgagatcgccaacaagcagaaggccaccctggtgtgcctggcaaggggcttctttcctgaccacgtggagctgtcctggtgggtgaatggcaaggaggtgcacagcggcgtgtccaccgatccacaggcctacaaggagtctaactacagctattgcctgagctccaggctgcgcgtgagcgccacattctggcacaaccctcgcaatcacttccggtgtcaggtgcagtttcacggcctgtctgaggaggacaagtggccagagggcagcccaaagccagtgacccagaatatctccgccgaggcatggggaagggcagattgcggcatcacatctgccagctatcaccagggcgtgctgagcgccaccatcctgtacgagatcctgctgggcaaggccacactgtatgccgtgctggtgtctggcctggtgctgatggccatggtgaagaagaagaacagc

SEQ ID NO: 77 - protein, 6 aa, synthetic construct
NYSPAY

SEQ ID NO: 78 - protein, 7 aa, synthetic construct
IRENEKE

SEQ ID NO: 79 - protein, 17 aa, synthetic construct
CALVSYGSGGSQGNLIF

SEQ ID NO: 80 - protein, 5 aa, synthetic construct
SGHNS

SEQ ID NO: 81 - protein, 6 aa, synthetic construct
FNNNVP

SEQ ID NO: 82 - protein, 13 aa, synthetic construct
CASSLSLNTEAFF

SEQ ID NO: 83 - protein, 155 aa, synthetic construct
MAFWLRRLGLHFRPHLGRRMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALVSYGSGGSQGNLIFGKGTKLSVKP

SEQ ID NO: 84 - protein, 132 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLSLNTEAFFGQGTRLTVV

SEQ ID NO: 85 - protein, 6 aa, synthetic construct
NSASQS

SEQ ID NO: 86 - protein, 6 aa, synthetic construct
VYSSGN

SEQ ID NO: 87 - protein, 14 aa, synthetic construct
CVVIPMDSSYKLIF

SEQ ID NO: 88 - protein, 5 aa, synthetic construct
MDHEN

SEQ ID NO: 89 - protein, 6 aa, synthetic construct
SYDVKM

SEQ ID NO: 90 - protein, 12 aa, synthetic construct
CASTWGEGEAFF

SEQ ID NO: 91 - protein, 132 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVIPMDSSYKLIFGSGTRLLVRP

SEQ ID NO: 92 - protein, 130 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASTWGEGEAFFGQGTRLTVV

SEQ ID NO: 93 - protein, 14 aa, synthetic construct
CAGQELIDGNQFYF

SEQ ID NO: 94 - protein, 5 aa, synthetic construct
SGHDY

SEQ ID NO: 95 - protein, 13 aa, synthetic construct
CASSPGSSYEQYF

SEQ ID NO: 96 - protein, 130 aa, synthetic construct
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGQELIDGNQFYFGTGTSLTVIP

SEQ ID NO: 97 - protein, 132 aa, synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSPGSSYEQYFGPGTRLTVT

SEQ ID NO: 98 - protein, 5 aa, synthetic construct
KALYS

SEQ ID NO: 99 - protein, 7 aa, synthetic construct
LLKGGEQ

SEQ ID NO: 100 - protein, 16 aa, synthetic construct
CGTEIRAGGGADGLTF

SEQ ID NO: 101 - protein, 13 aa, synthetic construct
CASSFFLGETQYF

SEQ ID NO: 102 - protein, 134 aa, synthetic construct
METLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEIRAGGGADGLTFGKGTHLIIQP

SEQ ID NO: 103 - protein, 132 aa, synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFFLGETQYFGPGTRLLVL

SEQ ID NO: 104 - protein, 7 aa, synthetic construct
NIATNDY

SEQ ID NO: 105 - protein, 5 aa, synthetic construct
GYKTK

SEQ ID NO: 106 - protein, 13 aa, synthetic construct
CLVGDGDNARLMF

SEQ ID NO: 107 - protein, 5 aa, synthetic construct
SGHDT

SEQ ID NO: 108 - protein, 6 aa, synthetic construct
YYEEEE

SEQ ID NO: 109 - protein, 16 aa, synthetic construct
CASSLPRMGSDTEAFF

SEQ ID NO: 110 - protein, 127 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDGDNARLMFGDGTQLVVKP

SEQ ID NO: 111 - protein, 134 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQYPNYSSELNVNALLLGDSALYLCASSLPRMGSDTEAFFGQGTRLTVV

SEQ ID NO: 112 - protein, 15 aa, synthetic construct
CLVGDMDGGYQKVTF

SEQ ID NO: 113 - protein, 15 aa, synthetic construct
CASSPTRLGYNEQFF

SEQ ID NO: 114 - protein, 129 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDMDGGYQKVTFGTGTKLQVIP

SEQ ID NO: 115 - protein, 133 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSPTRLGYNEQFFGPGTRLTVL

SEQ ID NO: 116 - protein, 13 aa, synthetic construct
CLVGDLDGQKLLF

SEQ ID NO: 117 - protein, 12 aa, synthetic construct
CASSLRTGEAFF

SEQ ID NO: 118 - protein, 127 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDLDGQKLLFARGTMLKVDL

SEQ ID NO: 119 - protein, 130 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLRTGEAFFGQGTRLTVV

SEQ ID NO: 120 - protein, 15 aa, synthetic construct
CLVGDMDSGNTPLVF

SEQ ID NO: 121 - protein, 5 aa, synthetic construct
LNHDA

SEQ ID NO: 122 - protein, 6 aa, synthetic construct
SQIVND

SEQ ID NO: 123 - protein, 16 aa, synthetic construct
CASSIDYRGIGETQYF

SEQ ID NO: 124 - protein, 129 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDMDSGNTPLVFGKGTRLSVIA

SEQ ID NO: 125 - protein, 134 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIDYRGIGETQYFGPGTRLLVL

SEQ ID NO: 126 - protein, 12 aa, synthetic construct
CAWEGGSEKLVF

SEQ ID NO: 127 - protein, 5 aa, synthetic construct
LGHNA

SEQ ID NO: 128 - protein, 6 aa, synthetic construct
YNFKEQ

SEQ ID NO: 129 - protein, 15 aa, synthetic construct
CASSSSGGRIYGYTF

SEQ ID NO: 130 - protein, 133 aa, synthetic construct
METLLGVSLVILWLQLARVNAQTVTQSQPEMSVQEAETVTLSCTYDTSESDYYLFWYKQPPSRQMILVIRQEAYKQQNATENRFSVNFQKAAKSFSLKISDSQLGDAAMYFCAWEGGSEKLVFGKGTKLTVNP

SEQ ID NO: 131 - protein, 133 aa, synthetic construct
MGTRLLCWAALCLLGAELTETGVTQTPRHLVMGMTNKKSLKCEQHLGHNAMYWYKQSAKKPLELMFVYNFKEQTENNSVPSRFSPECPNSSHLFLHLHTLQPEDSALYLCASSSSGGRIYGYTFGSGTRLTVV

SEQ ID NO: 132 - protein, 10 aa, Homo sapiens
GADGVGKSAL

SEQ ID NO: 133 - protein, 10 aa, Homo sapiens
GAVGVGKSAL

SEQ ID NO: 134 - protein, 9 aa, Homo sapiens
GAVGVGKSA

SEQ ID NO: 135 - protein, 10 aa, Homo sapiens
VVVGADGVGK

SEQ ID NO: 136 - protein, 4 aa, synthetic construct
SGSG

SEQ ID NO: 137 - protein, 8 aa, Influenza virus
GILGFVTL

(19) United States
(12) Patent Application Publication
(10) Pub. No.: US 2026/0116948 A1
(43) Pub. Date: Apr. 30, 2026

(54) T CELL RECEPTOR CONSTRUCTS AND USES THEREOF

(71) Applicant: BioNTech US Inc., Cambridge, MA (US)

(72) Inventors: Kendra Foley, Weymouth, MA (US); Porkodi Panneerselvam, Clarksburg, MD (US); Vikram Juneja, Newton, MA (US)

(21) Appl. No.: 19/233,443

(22) Filed: Jun. 10, 2025

Related U.S. Application Data
(63) Continuation of application No. PCT/US23/83613, filed on Dec. 12, 2023.
(60) Provisional application No. 63/387,148, filed on Dec. 13, 2022.

(51) Int. Cl. C07K 14/725; C07K 16/28; C07K 16/40

(52) U.S. Cl. CPC C07K 14/7051; C07K 16/2833; C07K 16/40; C07K 2317/31

(57) ABSTRACT

The present disclosure provides T cell receptors (TCRs) against peptide-MHC complexes, isolated nucleic acid molecules encoding TCRs against peptide-MHC complexes, T cells expressing TCRs against peptide-MHC complexes, and pharmaceutical compositions for use in the treatment of diseases.

### CROSS REFERENCE

This application is a continuation of International Application No. PCT/US2023/083613, filed Dec. 12, 2023, which claims the benefit of U.S. Provisional Application No. 63/387,148, filed Dec. 13, 2022, each of which is incorporated herein by reference in its entirety.

### SEQUENCE LISTING

The instant application contains a Sequence listing which has been submitted electronically in XML format and is herein incorporated by reference in its entirety. Said XML copy, created on Jan. 30, 2024, is named 50401-767_601_SL.xml and is 132,444 bytes in size.

### BACKGROUND

T cell receptors (TCRs) are members of the immunoglobulin superfamily and usually consist of two subunits, namely the α- and β-subunits. These possess one N-terminal immunoglobulin (Ig)-variable (V) domain, one Ig-constant (C) domain, a transmembrane/cell membrane-spanning region, and a short cytoplasmic tail at the C-terminal end. The variable domains of both the TCR α-chain and β-chain have three hypervariable or complementarity determining regions (CDRs), whereas the variable region of the β-chain has an additional area of hypervariability (HV4) that does not normally contact antigen and therefore is not considered a CDR.

CDR3 is the principal CDR responsible for recognizing processed antigen, although CDR1 of the alpha chain has also been shown to interact with the N-terminal part of the antigenic peptide, whereas CDR1 of the n-chain interacts with the C-terminal part of the peptide. CDR2 is thought to recognize the MHC. The constant domain of the TCR domain consists of short connecting sequences in which a cysteine residue forms disulfide bonds, which forms a link between the two chains. The affinity of TCR's for a specific antigen makes them valuable for several therapeutic approaches. For example, cancer patients, such as melanoma patients, can be effectively treated by using adoptive immunotherapy as TCRs are exquisitely sensitive for their antigen and can direct immune responses at tumor cells expressing their cognate antigen. Accordingly, there is a need for TCRs against peptide-MHC complexes for development of new and effective therapeutics.

### SUMMARY

Provided herein is a recombinant nucleic acid encoding a T cell receptor (TCR) comprising a TCR beta chain construct comprising a complementarity determining region 3 (CDR3) having an amino acid sequence set forth in SEQ ID NO: 6. In some embodiments, the TCR beta chain construct further comprises a complementarity determining region 1 (CDR1), wherein the CDR1 has an amino acid sequence set forth in SEQ ID NO: 4. In some embodiments, the TCR beta chain construct further comprises a complementarity determining region 2 (CDR2), wherein the CDR2 has an amino acid sequence set forth in SEQ ID NO:5. In some embodiments, the TCR beta chain construct comprises a variable region having at least 80% sequence identity to an amino acid sequence set forth in SEQ ID NO: 12. In some embodiments, the TCR beta chain construct comprises a variable region having at least 90% sequence identity to an amino acid sequence set forth in SEQ ID NO: 12. In some embodiments, the TCR beta chain construct comprises a variable region having at least 95%, 96%, 97%, 98% or 99% sequence identity to an amino acid sequence set forth in SEQ ID NO: 12. In some embodiments, the TCR beta chain construct comprises a variable region having an amino acid sequence set forth in SEQ ID NO: 12. In some embodiments, the recombinant nucleic acid further comprises a sequence encoding a TCR alpha chain construct having a CDR1, a CDR2, and a CDR3, wherein, the CDR1 has an amino acid sequence set forth in SEQ ID NO: 1; the CDR2 has an amino acid sequence set forth in SEQ ID NO: 2; and the CDR3 has an amino acid sequence set forth in SEQ ID NO: 3. In some embodiments, the recombinant nucleic acid comprises: (a) a sequence having at least 80% sequence identity with SEQ ID NOs: 10 or 11; and (b) a sequence having at least 80% sequence identity with SEQ ID NOs: 7 or 8. In some embodiments, the TCR alpha chain construct comprises a variable region having at least 80% sequence identity to an amino acid sequence set forth in SEQ ID NO: 9. In some embodiments, the TCR alpha chain construct comprises a variable region having at least 90% sequence identity to an amino acid sequence set forth in SEQ ID NO: 9. In some embodiments, the TCR alpha chain construct comprises a variable region having at least 95%, 96%, 97%, 98% or 99% sequence identity to an amino acid sequence set forth in SEQ ID NO: 9. In some embodiments, the TCR alpha chain construct comprises a variable region having an amino acid sequence set forth in SEQ ID NO: 9. In some embodiments, the TCR comprises: (a) a beta chain having an amino acid sequence set forth in SEQ ID NOs: 14 or 70, or an amino acid sequence that is at least 80% identical to SEQ ID NOs: 14 or 70, (b) an alpha chain having an amino acid sequence set forth in SEQ ID NOs: 13 or 69, or an amino acid sequence that is at least 80% identical to SEQ ID NOs: 13 or 69. In some embodiments, the TCR binds to an epitope from human RAS comprising a mutation G12V. In some embodiments, the epitope from human RAS comprising the mutation G12V is SEQ ID NOs: 43 or 44. In some embodiments, the TCR binds to a complex comprising (i) the epitope from human RAS comprising the mutation G12V and (ii) an MHC protein encoded by an HLA-A11:01 allele.

Provided herein is a recombinant nucleic acid encoding a T cell receptor (TCR) comprising a TCR beta chain construct comprising a complementarity determining region 3 (CDR3) having an amino acid sequence set forth in SEQ ID NO: 20. In some embodiments, the TCR beta chain construct further comprises a complementarity determining region 1 (CDR1), wherein the CDR1 has an amino acid sequence set forth in SEQ ID NO: 18. In some embodiments, the TCR beta chain construct further comprises a complementarity determining region 2 (CDR2), wherein the CDR2 has an amino acid sequence set forth in SEQ ID NO: 19. In some embodiments, the TCR beta chain construct comprises a variable region having at least 80% sequence identity to an amino acid sequence set forth in SEQ ID NO: 26. In some embodiments, the TCR beta chain construct comprises a variable region having at least 90% sequence identity to an amino acid sequence set forth in SEQ ID NO: 26. In some embodiments, the TCR beta chain construct comprises a variable region having at least 95%, 96%, 97%, 98% or 99% sequence identity to an amino acid sequence set forth in SEQ ID NO: 26. In some embodiments, the TCR beta chain construct comprises a variable region having an amino acid sequence set forth in SEQ ID NO: 26. In some embodiments, the recombinant nucleic acid further comprises a sequence encoding a TCR alpha chain construct having a CDR1, a CDR2, and a CDR3, wherein, the CDR1 has an amino acid sequence set forth in SEQ ID NO: 15; the CDR2 has an amino acid sequence set forth in SEQ ID NO: 16; and the CDR3 has an amino acid sequence set forth in SEQ ID NO: 17. In some embodiments, the recombinant nucleic acid comprises: (a) a sequence having at least 80% sequence identity with SEQ ID NOs: 24 or 25; and (b) a sequence having at least 80% sequence identity with SEQ ID NOs: 21 or 22. In some embodiments, the TCR alpha chain construct comprises a variable region having at least 80% sequence identity to an amino acid sequence set forth in SEQ ID NO: 23. In some embodiments, the TCR alpha chain construct comprises a variable region having at least 90% sequence identity to an amino acid sequence set forth in SEQ ID NO: 23. In some embodiments, the TCR alpha chain construct comprises a variable region having at least 95%, 96%, 97%, 98% or 99% sequence identity to an amino acid sequence set forth in SEQ ID NO: 23. In some embodiments, the TCR alpha chain construct comprises a variable region having an amino acid sequence set forth in SEQ ID NO: 23. In some embodiments, the TCR comprises: (a) a beta chain having an amino acid sequence set forth in SEQ ID NOs: 28 or 72, or an amino acid sequence that is at least 80% identical to SEQ ID NOs: 28 or 72, (b) an alpha chain having an amino acid sequence set forth in SEQ ID NOs: 27 or 71, or an amino acid sequence that is at least 80% identical to SEQ ID NOs: 27 or 71. In some embodiments, the TCR binds to an epitope from human RAS comprising a mutation G12V. In some embodiments, the epitope from human RAS comprising the mutation G12V is SEQ ID NO: 45. In some embodiments, the TCR binds to a complex comprising (i) the epitope from human RAS comprising the mutation G12V and (ii) an MHC protein encoded by an HLA-C01:02 allele.

Provided herein is a recombinant nucleic acid encoding a T cell receptor (TCR) comprising a TCR beta chain construct comprising a complementarity determining region 3 (CDR3) having an amino acid sequence set forth in SEQ ID NO: 34. In some embodiments, the TCR beta chain construct further comprises a complementarity determining region 1 (CDR1), wherein the CDR1 has an amino acid sequence set forth in SEQ ID NO: 32. In some embodiments, the TCR beta chain construct further comprises a complementarity determining region 2 (CDR2), wherein the CDR2 has an amino acid sequence set forth in SEQ ID NO: 33. In some embodiments, the TCR beta chain construct comprises a variable region having at least 80% sequence identity to an amino acid sequence set forth in SEQ ID NO: 40. In some embodiments, the TCR beta chain construct comprises a variable region having at least 90% sequence identity to an amino acid sequence set forth in SEQ ID NO: 40. In some embodiments, the TCR beta chain construct comprises a variable region having at least 95%, 96%, 97%, 98% or 99% sequence identity to an amino acid sequence set forth in SEQ ID NO: 40. In some embodiments, the TCR beta chain construct comprises a variable region having an amino acid sequence set forth in SEQ ID NO: 40. In some embodiments, the recombinant nucleic acid further comprises a sequence encoding a TCR alpha chain construct having a CDR1, a CDR2, and a CDR3, wherein, the CDR1 has an amino acid sequence set forth in SEQ ID NO: 29; the CDR2 has an amino acid sequence set forth in SEQ ID NO: 30; and, the CDR3 has an amino acid sequence set forth in SEQ ID NO: 31. In some embodiments, the recombinant nucleic acid comprises: (a) a sequence having at least 80% sequence identity with SEQ ID NOs: 38 or 39; and (b) a sequence having at least 80% sequence identity with SEQ ID NOs: 35 or 36. In some embodiments, the TCR alpha chain construct comprises a variable region having at least 80% sequence identity to an amino acid sequence set forth in SEQ ID NO: 37. In some embodiments, the TCR alpha chain construct comprises a variable region having at least 90% sequence identity to an amino acid sequence set forth in SEQ ID NO: 37. In some embodiments, the TCR alpha chain construct comprises a variable region having at least 95%, 96%, 97%, 98% or 99% sequence identity to an amino acid sequence set forth in SEQ ID NO: 37. In some embodiments, the TCR alpha chain construct comprises a variable region having an amino acid sequence set forth in SEQ ID NO: 37. In some embodiments, the TCR comprises: (a) a beta chain having an amino acid sequence set forth in SEQ ID NOs: 41 or 74, or an amino acid sequence that is at least 80% identical to SEQ ID NOs: 41 or 74, (b) an alpha chain having an amino acid sequence set forth in SEQ ID NOs: 42 or 73, or an amino acid sequence that is at least 80% identical to SEQ ID NOs: 42 or 73. In some embodiments, the TCR binds to an epitope from human RAS comprising a mutation G12V. In some embodiments, the epitope from human RAS comprising the mutation G12V is SEQ ID NO: 45. In some embodiments, the TCR binds to a complex comprising (i) the epitope from human RAS comprising the mutation G12V and (ii) an MHC protein encoded by an HLA-C01:02 allele.

In some embodiments, the recombinant nucleic acid is a vector. In some embodiments, the epitope has a length of from 8-25 amino acids. In some embodiments, the epitope comprises a mutation that differs from a corresponding wild-type epitope by at least one amino acid. In some embodiments, the epitope binds to the human MHC with a greater affinity than a corresponding wild-type epitope. In some embodiments, the epitope binds to the human MHC with a K_{D} or an IC₅₀ less than 500 nM, 250 nM, 150 nM, 100 nM, 50 nM, 25 nM, or 10 nM. In some embodiments, the mutation is not present in non-cancer cells of a subject. In some embodiments, the TCR binds to a MHC-peptide complex with a K_{D} or an IC₅₀ of less than 500 nM, 250 nM, 150 nM, 100 nM, 50 nM, 25 nM, or 10 nM. In some embodiments, the recombinant nucleic acid is operably linked to a promoter.

Provided herein is a cell comprising a recombinant nucleic acid described herein. In some embodiments, the cell is a CD4+ T cell. In some embodiments, the cell is a CD8+ T cell. In some embodiments, the cell is isolated from a subject having a RAS mutation.

Provided herein is a pharmaceutical composition comprising: (a) a recombinant nucleic acid described herein, or a cell described herein; and (b) a pharmaceutically acceptable excipient or diluent. In some embodiments, the pharmaceutical composition further comprises an immunomodulatory agent or an adjuvant. In some embodiments, the adjuvant is poly I:C. In some embodiments, the pharmaceutical composition is for use in treating an immune disease or cancer.

Provided herein is use of a pharmaceutical composition described herein, for treating an immune disease or cancer.

Provided herein is a method of treating a subject having a disease or condition, comprising administering to the subject a pharmaceutical composition described herein.

Provided herein is a method of treating a subject with cancer comprising administering to the subject a pharmaceutical composition described herein.

Provided herein is a method of identifying a subject with cancer as a candidate for a therapeutic, the method comprising determining the subject as a subject that expresses a protein encoded by an HLA-A11:01 allele or an HLA C01:02 allele, wherein the therapeutic is a pharmaceutical composition described herein.

Provided herein is a nucleic acid encoding a TCR, wherein the TCR binds to a complex comprising (i) an epitope from human RAS comprising a mutation G12V and (ii) an MHC protein encoded by an HLA-A11:01 allele or an HLA C01:02 allele, wherein a complementarity determining region 3 (CDR3) of the TCR does not comprise a sequence of any one of SEQ ID NOs: 46-68.

Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-A68:01 allele, wherein the mutated epitope from human RAS is characterized by a G12V mutation. In some embodiments, the TCR beta chain construct comprises a complementarity determining region 3 (CDR3), wherein the CDR3 has an amino acid sequence of SEQ ID NO: 82. In some embodiments, the TCR beta chain construct comprises a complementarity determining region 1 (CDR1), wherein the CDR1 has an amino acid sequence of SEQ ID NO: 80. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 84. In some embodiments, the TCR beta chain construct comprises a variable region having at least 90% sequence identity to an amino acid sequence set forth in SEQ ID NO: 84. In some embodiments, the TCR beta chain construct comprises a variable region having at least 95%, 96%, 98%, or 99% sequence identity to an amino acid sequence set forth in SEQ ID NO: 84. In some embodiments, the TCR beta chain construct comprises a variable region having an amino acid sequence set forth in SEQ ID NO: 84. In some embodiments, the TCR further comprises a TCR alpha chain construct having a CDR1, a CDR2, and a CDR3, wherein, the CDR1 has an amino acid sequence set forth in SEQ ID NO: 77; the CDR2 has an amino acid sequence set forth in SEQ ID NO: 78; and, the CDR3 has an amino acid sequence set forth in SEQ ID NO: 79. In some embodiments, the TCR alpha chain construct comprises a variable region having at least 80% sequence identity to an amino acid sequence set forth in SEQ ID NO: 83. In some embodiments, the TCR alpha chain construct comprises a variable region having at least 90% sequence identity to an amino acid sequence set forth in SEQ ID NO: 83. In some embodiments, the TCR alpha chain construct comprises a variable region having at least 95%, 96%, 98%, or 99% sequence identity to an amino acid sequence set forth in SEQ ID NO: 83. In some embodiments, the TCR alpha chain construct comprises a variable region having an amino acid sequence set forth in SEQ ID NO: 83. In some embodiments, the epitope from human RAS comprising the mutation G12V is SEQ ID NO: 43 or 44.

Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-C03:03 allele, wherein the mutated epitope from human RAS is characterized by a G12V mutation. Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-C03:04 allele, wherein the mutated epitope from human RAS is characterized by a G12V mutation. Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-C03:03 allele and a human MHC encoded by an HLA-C03:04 allele, wherein the mutated epitope from human RAS is characterized by a G12V mutation. In some embodiments, the TCR binds to (i) the mutated epitope from human RAS in complex with the human MHC encoded by the HLA-C03:03 allele with a K_{D} of at most 500 nM, at most 250 nM, at most 50 nM, at most 25 nM, at most 10 nM, or at most 5 nM; and (ii) the mutated epitope from human RAS in complex with the human MHC encoded by the HLA-C03:04 allele with a K_{D} of at most 500 nM, at most 250 nM, at most 50 nM, at most 25 nM, at most 10 nM, or at most 5 nM. In some embodiments, the TCR beta chain construct comprises a complementarity determining region 3 (CDR3), wherein the CDR3 has an amino acid sequence of SEQ ID NO: 109. In some embodiments, the TCR beta chain construct comprises a complementarity determining region 1 (CDR1), wherein the CDR1 has an amino acid sequence of SEQ ID NO: 107. In some embodiments, the TCR beta chain construct comprises a complementarity determining region 2 (CDR2), wherein the CDR2 has an amino acid sequence of SEQ ID NO: 108. In some embodiments, the TCR beta chain construct comprises a variable region having at least 80% sequence identity to an amino acid sequence set forth in SEQ ID NO: 111. In some embodiments, the TCR beta chain construct comprises a variable region having at least 90% sequence identity to an amino acid sequence set forth in SEQ ID NO: 111. In some embodiments, the TCR beta chain construct comprises a variable region having at least 95%, 96%, 98%, or 99% sequence identity to an amino acid sequence set forth in SEQ ID NO: 111. In some embodiments, the TCR beta chain construct comprises a variable region having an amino acid sequence set forth in SEQ ID NO: 111. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 106. In some embodiments, the TCR alpha chain construct comprises a variable region having at least 80% sequence identity to an amino acid sequence set forth in SEQ ID NO: 110. In some embodiments, the TCR alpha chain construct comprises a variable region having at least 90% sequence identity to an amino acid sequence set forth in SEQ ID NO: 110. In some embodiments, the TCR alpha chain construct comprises a variable region at least 95%, 96%, 98%, or 99% sequence identity to an amino acid sequence set forth in SEQ ID NO: 110. In some embodiments, the TCR alpha chain construct comprises a variable region having an amino acid sequence set forth in SEQ ID NO: 110.

In some embodiments, the TCR beta chain construct comprises a complementarity determining region 3 (CDR3), wherein the CDR3 has an amino acid sequence of SEQ ID NO: 113. In some embodiments, the TCR beta chain construct comprises a complementarity determining region 1 (CDR1), wherein the CDR1 has an amino acid sequence of SEQ ID NO: 88.

In some embodiments, the TCR beta chain construct comprises a complementarity determining region 2 (CDR2), wherein the CDR2 has an amino acid sequence of SEQ ID NO: 89. In some embodiments, the TCR beta chain construct comprises a variable region having at least 80% sequence identity to an amino acid sequence set forth in SEQ ID NO: 115. In some embodiments, the TCR beta chain construct comprises a variable region having at least 90% sequence identity to an amino acid sequence set forth in SEQ ID NO: 115. In some embodiments, the TCR beta chain construct comprises a variable region having at least 95%, 96%, 98%, or 99% sequence identity to an amino acid sequence set forth in SEQ ID NO: 115. In some embodiments, the TCR beta chain construct comprises a variable region having an amino acid sequence set forth in SEQ ID NO: 115. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 112. In some embodiments, the TCR alpha chain construct comprises a variable region having at least 80% sequence identity to an amino acid sequence set forth in SEQ ID NO: 114. In some embodiments, the TCR alpha chain construct comprises a variable region having at least 90% sequence identity to an amino acid sequence set forth in SEQ ID NO: 114. In some embodiments, the TCR alpha chain construct comprises a variable region having at least 95%, 96%, 98%, or 99% sequence identity to an amino acid sequence set forth in SEQ ID NO: 114. In some embodiments, the TCR alpha chain construct comprises a variable region having an amino acid sequence set forth in SEQ ID NO: 114.

In some embodiments, the TCR beta chain construct comprises a complementarity determining region 3 (CDR3), wherein the CDR3 has an amino acid sequence of SEQ ID NO: 117. In some embodiments, the TCR beta chain construct comprises a complementarity determining region 1 (CDR1), wherein the CDR1 has an amino acid sequence of SEQ ID NO: 107. In some embodiments, the TCR beta chain construct comprises a complementarity determining region 2 (CDR2), wherein the CDR2 has an amino acid sequence of SEQ ID NO: 108. In some embodiments, the TCR beta chain construct comprises a variable region having at least 80% sequence identity to an amino acid sequence set forth in SEQ ID NO: 119. In some embodiments, the TCR beta chain construct comprises a variable region having at least 90% sequence identity to an amino acid sequence set forth in SEQ ID NO: 119. In some embodiments, the TCR beta chain construct comprises a variable region having at least 95%, 96%, 98%, or 99% sequence identity to an amino acid sequence set forth in SEQ ID NO: 119. In some embodiments, the TCR beta chain construct comprises a variable region having an amino acid sequence set forth in SEQ ID NO: 119. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 116. In some embodiments, the TCR alpha chain construct comprises a variable region having at least 80% sequence identity to an amino acid sequence set forth in SEQ ID NO: 118. In some embodiments, the TCR alpha chain construct comprises a variable region having at least 90% sequence identity to an amino acid sequence set forth in SEQ ID NO: 118. In some embodiments, the TCR alpha chain construct comprises a variable region having at least 95%, 96%, 98%, or 99% sequence identity to an amino acid sequence set forth in SEQ ID NO: 118. In some embodiments, the TCR alpha chain construct comprises a variable region having an amino acid sequence set forth in SEQ ID NO: 118.

In some embodiments, the TCR beta chain construct comprises a complementarity determining region 3 (CDR3), wherein the CDR3 has an amino acid sequence of SEQ ID NO: 123. In some embodiments, the TCR beta chain construct comprises a complementarity determining region 1 (CDR1), wherein the CDR1 has an amino acid sequence of SEQ ID NO: 121. In some embodiments, the TCR beta chain construct comprises a complementarity determining region 2 (CDR2), wherein the CDR2 has an amino acid sequence of SEQ ID NO: 122. In some embodiments, the TCR beta chain construct comprises a variable region having at least 80% sequence identity to an amino acid sequence set forth in SEQ ID NO: 125. In some embodiments, the TCR beta chain construct comprises a variable region having at least 90% sequence identity to an amino acid sequence set forth in SEQ ID NO: 125. In some embodiments, the TCR beta chain construct comprises a variable region having at least 95%, 96%, 98%, or 99% sequence identity to an amino acid sequence set forth in SEQ ID NO: 125. In some embodiments, the TCR beta chain construct comprises a variable region having an amino acid sequence set forth in SEQ ID NO: 125. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 120. In some embodiments, the TCR alpha chain construct comprises a variable region having at least 80% sequence identity to an amino acid sequence set forth in SEQ ID NO: 124. In some embodiments, the TCR alpha chain construct comprises a variable region having at least 90% sequence identity to an amino acid sequence set forth in SEQ ID NO: 124. In some embodiments, the TCR alpha chain construct comprises a variable region having at least 95%, 96%, 98%, or 99% sequence identity to an amino acid sequence set forth in SEQ ID NO: 124. In some embodiments, the TCR alpha chain construct comprises a variable region having an amino acid sequence set forth in SEQ ID NO: 124. In some embodiments, the epitope from human RAS comprising the mutation G12V is SEQ ID NO: 133 or 134.

Provided herein is a recombinant nucleic acid encoding a T cell receptor (TCR) comprising a TCR beta chain construct comprising a complementarity determining region 3 (CDR3) having an amino acid sequence set forth in SEQ ID NO: 129. In some embodiments, the TCR beta chain construct comprises a complementarity determining region 1 (CDR1), wherein the CDR1 has an amino acid sequence of SEQ ID NO: 127. In some embodiments, the TCR beta chain construct comprises a complementarity determining region 2 (CDR2), wherein the CDR2 has an amino acid sequence of SEQ ID NO: 128. In some embodiments, the TCR beta chain construct comprises a variable region having at least 80% sequence identity to an amino acid sequence set forth in SEQ ID NO: 131. In some embodiments, the TCR beta chain construct comprises a variable region having at least 90% sequence identity to an amino acid sequence set forth in SEQ ID NO: 131. In some embodiments, the TCR beta chain construct comprises a variable region having at least 95%, 96%, 98%, or 99% sequence identity to an amino acid sequence set forth in SEQ ID NO: 131. In some embodiments, the TCR beta chain construct comprises a variable region having an amino acid sequence set forth in SEQ ID NO: 131. In some embodiments, the TCR further comprises a TCR alpha chain construct having a CDR1, a CDR2, and a CDR3, wherein, the CDR1 has an amino acid sequence set forth in SEQ ID NO: 29; the CDR2 has an amino acid sequence set forth in SEQ ID NO: 30; and, the CDR3 has an amino acid sequence set forth in SEQ ID NO: 126. In some embodiments, the TCR alpha chain construct comprises a variable region having at least 80% sequence identity to an amino acid sequence set forth in SEQ ID NO: 130. In some embodiments, the TCR alpha chain construct comprises a variable region having at least 90% sequence identity to an amino acid sequence set forth in SEQ ID NO: 130. In some embodiments, the TCR alpha chain construct comprises a variable region having at least 95%, 96%, 98%, or 99% sequence identity to an amino acid sequence set forth in SEQ ID NO: 130. In some embodiments, the TCR alpha chain construct comprises a variable region having an amino acid sequence set forth in SEQ ID NO: 130. In some embodiments, the TCR binds to an epitope from human RAS comprising a mutation G12D. In some embodiments, the epitope from human RAS comprising the mutation G12D is SEQ ID NO: 135. In some embodiments, the TCR binds to a complex comprising (i) the epitope from human RAS comprising the mutation G12D and (ii) an MHC protein encoded by an HLA-A11:01 allele.

Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-C05:01 allele with a K_{D} of at most 1000 nM, wherein the mutated epitope from human RAS is characterized by a G12D mutation. Provided herein is a recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-C05:01 allele with a K_{D} of 1000 nM or more, wherein the mutated epitope from human RAS is characterized by a G12D mutation. In some embodiments, the TCR beta chain construct comprises a complementarity determining region 3 (CDR3), wherein the CDR3 has an amino acid sequence of SEQ ID NO: 90. In some embodiments, the TCR beta chain construct comprises a complementarity determining region 1 (CDR1), wherein the CDR1 has an amino acid sequence of SEQ ID NO: 88. In some embodiments, the TCR beta chain construct comprises a complementarity determining region 2 (CDR2), wherein the CDR2 has an amino acid sequence of SEQ ID NO: 89. In some embodiments, the TCR beta chain construct comprises a variable region having at least 80% sequence identity to an amino acid sequence set forth in SEQ ID NO: 92. In some embodiments, the TCR beta chain construct comprises a variable region having at least 90% sequence identity to an amino acid sequence set forth in SEQ ID NO: 92. In some embodiments, the TCR beta chain construct comprises a variable region having at least 95%, 96%, 98%, or 99% sequence identity to an amino acid sequence set forth in SEQ ID NO: 92. In some embodiments, the TCR beta chain construct comprises a variable region having an amino acid sequence set forth in SEQ ID NO: 92. In some embodiments, the TCR further comprises a TCR alpha chain construct having a CDR1, a CDR2, and a CDR3, wherein, the CDR1 has an amino acid sequence set forth in SEQ ID NO: 85; the CDR2 has an amino acid sequence set forth in SEQ ID NO: 86; and, the CDR3 has an amino acid sequence set forth in SEQ ID NO: 87. In some embodiments, the TCR alpha chain construct comprises a variable region having at least 80% sequence identity to an amino acid sequence set forth in SEQ ID NO: 91. In some embodiments, the TCR alpha chain construct comprises a variable region having at least 90% sequence identity to an amino acid sequence set forth in SEQ ID NO: 91. In some embodiments, the TCR alpha chain construct comprises a variable region having at least 95%, 96%, 98%, or 99% sequence identity to an amino acid sequence set forth in SEQ ID NO: 91. In some embodiments, the TCR alpha chain construct comprises a variable region having an amino acid sequence set forth in SEQ ID NO: 91.

In some embodiments, the TCR beta chain construct comprises a complementarity determining region 3 (CDR3), wherein the CDR3 has an amino acid sequence of SEQ ID NO: 95. In some embodiments, the TCR beta chain construct comprises a complementarity determining region 1 (CDR1), wherein the CDR1 has an amino acid sequence of SEQ ID NO: 94. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 97. In some embodiments, the TCR beta chain construct comprises a variable region having at least 90% sequence identity to an amino acid sequence set forth in SEQ ID NO: 97. In some embodiments, the TCR beta chain construct comprises a variable region having at least 95%, 96%, 98%, or 99% sequence identity to an amino acid sequence set forth in SEQ ID NO: 97. In some embodiments, the TCR beta chain construct comprises a variable region having an amino acid sequence set forth in SEQ ID NO: 97. In some embodiments, the TCR further comprises a TCR alpha chain construct having a CDR1, a CDR2, and a CDR3, wherein, the CDR1 has an amino acid sequence set forth in SEQ ID NO: 15; the CDR2 has an amino acid sequence set forth in SEQ ID NO: 16; and, the CDR3 has an amino acid sequence set forth in SEQ ID NO: 93. In some embodiments, the TCR alpha chain construct comprises a variable region having at least 80% sequence identity to an amino acid sequence set forth in SEQ ID NO: 96. In some embodiments, the TCR alpha chain construct comprises a variable region having at least 90% sequence identity to an amino acid sequence set forth in SEQ ID NO: 96. In some embodiments, the TCR alpha chain construct comprises a variable region having at least 95%, 96%, 98%, or 99% sequence identity to an amino acid sequence set forth in SEQ ID NO: 96. In some embodiments, the TCR alpha chain construct comprises a variable region having an amino acid sequence set forth in SEQ ID NO: 96.

In some embodiments, the TCR beta chain construct comprises a complementarity determining region 3 (CDR3), wherein the CDR3 has an amino acid sequence of SEQ ID NO: 101. In some embodiments, the TCR beta chain construct comprises a complementarity determining region 1 (CDR1), wherein the CDR1 has an amino acid sequence of SEQ ID NO: 94. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 103. In some embodiments, the TCR beta chain construct comprises a variable region having at least 90% sequence identity to an amino acid sequence set forth in SEQ ID NO: 103. In some embodiments, the TCR beta chain construct comprises a variable region having at least 95%, 96%, 98%, or 99% sequence identity to an amino acid sequence set forth in SEQ ID NO: 103. In some embodiments, the TCR beta chain construct comprises a variable region having an amino acid sequence set forth in SEQ ID NO: 103. In some embodiments, the TCR further comprises a TCR alpha chain construct having a CDR1, a CDR2, and a CDR3, wherein, the CDR1 has an amino acid sequence set forth in SEQ ID NO: 98; the CDR2 has an amino acid sequence set forth in SEQ ID NO: 99; and, the CDR3 has an amino acid sequence set forth in SEQ ID NO: 100. In some embodiments, the TCR alpha chain construct comprises a variable region having at least 80% sequence identity to an amino acid sequence set forth in SEQ ID NO: 102. In some embodiments, the TCR alpha chain construct comprises a variable region having at least 90% sequence identity to an amino acid sequence set forth in SEQ ID NO: 102. In some embodiments, the TCR alpha chain construct comprises a variable region having at least 95%, 96%, 98%, or 99% sequence identity to an amino acid sequence set forth in SEQ ID NO: 102. In some embodiments, the TCR alpha chain construct comprises a variable region having an amino acid sequence set forth in SEQ ID NO: 102. In some embodiments, the epitope from human RAS comprising the mutation G12D is SEQ ID NO: 132.

In some embodiments, the TCR specifically binds to the mutated epitope with a EC₅₀ of at least 1000 nM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a workflow for antigen-specific TCR identification and analysis. PBMCs from healthy donors can be stimulated with the antigen of interest, after which antigen-specific T cells can be identified with peptide-MHC multimers (left). Antigen-specific T cells can be isolated and the TCR can be sequenced and analyzed (middle left). The TCR can be synthesized and expressed in a cell line or PBMCs, and the specificity can be again confirmed by peptide-MHC multimer (middle right). The TCR-expressing cell line or PBMCs can then be co-cultured with antigen-expressing cell lines to confirm the functionality of the TCR (right).

FIG. 2 an example schematic of an antigen-specific CD8⁺ T cell expansion. PBMCs can be stimulated with the antigen of interest and cytokines. After expansion antigen-specific CD8⁺ T cells can be identified with peptide-MHC multimers.

FIG. 3A is an example schematic of recombinant TCR constructs and vector design for expression of the TCR constructs in cells.

FIG. 3B is an example schematic of a viral vector encoding recombinant TCRs for transduction or transfection into cells.

FIG. 4A depicts an example flow cytometry analysis of RAS antigen specific CD8⁺ T cell expansion in response to stimulation with a RAS peptide.

FIG. 4B depicts an example flow cytometry analysis of RAS antigen specific Jurkat cells after sorting of top 10% of multimer positive cells expressing a recombinant TCR after puromycin selection.

FIG. 5 depicts an example flow cytometry analysis of RAS-peptide-HLA-A11:01 complex specific CD8⁺ T cell expansion in response to stimulation with RAS G12V peptide (left), RAS-peptide-HLA-A11:01 complex specific CD8⁺ T cell expansion in response to stimulation with RAS G12C peptide (middle), and RAS-peptide-HLA-A11:01 complex specific CD8⁺ T cell expansion in response to stimulation with RAS G12D peptide (right).

FIG. 6 depicts example flow cytometry analyses of RAS-peptide-HLA-A11:01 complex specific CD8⁺ T cell expansion in response to stimulation with a G12V or G12C mutant RAS peptides (left), TCR clone abundance analysis after sequencing the TCR from pooled hits (middle), and confirmation of mutant versus wild-type specificity of the top 2 retrieved clones after recombinant TCR expression in Jurkat cells (right).

FIG. 7 depicts an example flow cytometry analysis of Jurkat cells transduced to express RAS TCR-1.

FIG. 8 depicts an example flow cytometry analysis of peripheral blood mononuclear cells transduced to express RAS TCR-1.

FIG. 9 depicts an example flow cytometry analysis of electroporated Jurkat cells that express RAS TCR-2 or RAS TCR-3.

FIG. 10 depicts experimental results of TCR functional assays to assess the specificity and avidity of RAS TCR-1. Graphs showing CD69 activation after co-culturing RAS TCR-transduced Jurkat cells with A375 cells expressing HLA-A11:01 loaded with either RAS wild-type (far right data point of each panel) or RAS-mutant 9mer peptide (left) or increasing amounts of RAS-mutant 10mer peptide (right).

FIG. 11 depicts experimental results of TCR functional assays to assess the specificity and avidity of RAS TCR-2 and RAS TCR-3. Graphs showing CD69 activation after co-culturing RAS TCRs-transduced Jurkat cells with K562 cells expressing HLA-C01:02 loaded with either RAS wild-type (far right data point of each panel) or RAS-mutant peptides.

FIG. 12 depicts experimental results of a cytotoxicity assay in which PBMCs transduced with an irrelevant TCR or RAS TCR-1 were co-cultured with SW620 (RAS G12V+) target tumor cells expressing HLA-A11:01 and GFP over an increasing range of CD4+ cells to target ratios (E:T). SW620 target tumor cells cultured alone or co-cultured with PBMCs transduced with the irrelevant TCR was used as negative control. Graph depicts the growth of the target cells over 100 hours (X-axis) as measured by GFP signal, as reflected by total green object area of the images of the cultures (Y-axis).

FIG. 13 depicts experimental results of a cytotoxicity assay in which CD4+ T cells, isolated from PBMCs transduced with an irrelevant TCR or RAS TCR-1, were co-cultured with SW620 (RAS G12V+) target tumor cells expressing HLA-A11:01 and GFP over an increasing range of PBMC to target ratios. SW620 target tumor cells cultured alone or without any PBMCs was used as a negative control. Graph depicts the growth of the target cells over 72 hours (X-axis) as measured by GFP signal, as reflected by total green object area of the images of the cultures (Y-axis).

FIG. 14 depicts exemplary data showing percent lysis of the tumor cell line SNG-M over time following treatment with PBMCs transduced with an irrelevant TCR or RAS TCR-1.

FIG. 15 depicts exemplary data showing tumor volume in mice subcutaneously inoculated with the tumor line A375-HLA-A11:01-KRAS G12V over time following treatment with PBMCs transduced with an irrelevant chimeric antigen receptor (CAR) or RAS TCR-1. Mice treated with RAS TCR-1 transduced T cells exhibited tumor regression compared to the PBS and irrelevant transduced T cell controls.

FIG. 16 depicts exemplary data showing functional avidity of RAS TCR-2 and RAS TCR-3.

FIG. 17A depicts exemplary data showing percent lysis of the colorectal cancer cell line SW620 engineered to express HLA C*01:02 following treatment with PBMCs transduced with RAS TCR-2 or RAS TCR-3 at the indicated effector to target cell ratios. % cytolysis was calculated based on tumor cells cocultured with non-transduced T cells.

FIG. 17B depicts exemplary data showing percent lysis of the pancreatic tumor cell line Panc 03.27 following treatment with PBMCs transduced with RAS TCR-2 or RAS TCR-3 at the indicated effector to target cell ratios. % cytolysis was calculated based on tumor cells cocultured with non-transduced T cells.

FIG. 18 depicts exemplary data showing percentage of CD69+ cells in the engineered cells after transduction with different concentrations of RAS TCR-5, RAS TCR-6, or RAS TCR-7 that bind to G12D RAS in complex with an MHC encoded by the HLA-C05:01 allele.

### DETAILED DESCRIPTION

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. The details of one or more particular embodiments are set forth in the description below.

### I. Definitions

Unless otherwise stated, the term “TCR” should be understood to encompass full TCRs as well as antigen-binding portions or antigen-binding fragments (also called MHC-peptide binding fragments) thereof. In some embodiments, the TCR is an intact or full-length TCR. In some embodiments, the TCR is an antigen-binding portion that is less than a full-length TCR but that binds to a specific antigenic peptide bound to (e.g., in the context of) an MHC molecule, e.g., an MHC-peptide complex. In some cases, an antigen-binding portion or fragment of a TCR can contain only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable α chain and variable β chain of a TCR, sufficient to form a binding site for binding to a specific MHC-peptide complex, such as generally where each chain contains three complementarity determining regions. Polypeptides or proteins having a binding domain which is an antigen-binding domain or is homologous to an antigen-binding domain are included. Complementarity determining region (CDR) grafted TCRs and other humanized TCRs (including CDR modifications and framework region modifications) are also contemplated by these terms. It should be noted that while reference may be made only to immunoglobulin chains (e.g., heavy chains and lights chains), the disclosed invention can be applied to multiple other different types of paired sequences, e.g., T cell receptor chain pairs (TCRα and TCRβ chains and TCRγ and TCRδ chains), and is not limited to immunoglobulins.

The terms “complementarity determining region,” and “CDR,” synonymous with “hypervariable region” or “HVR,” are known in the art to refer to non-contiguous sequences of amino acids within TCR variable regions, which confer specificity and/or binding affinity to an MHC-peptide complex. In general, there are three CDRs in each alpha chain variable region (CDR-H1, CDR-H2, CDR-H3) and three CDRs in each beta chain variable region (CDR-L1, CDR-L2, CDR-L3). “Framework regions” and “FR” are known in the art to refer to the non-CDR portions of the variable regions of the alpha and beta chains. In general, there are four FRs in each full-length alpha chain variable region (FR-H1, FR-H2, FR-H3, and FR-H4), and four FRs in each full-length beta chain variable region (FR-L1, FR-L2, FR-L3, and FR-L4).

The term “variable region” or “variable domain” refers to the domain of a TCR alpha, beta, gamma, or delta chain, that is involved in binding the TCR to antigen-MHC complexes. The variable domains of the alpha chain and beta chain (Va and VD, respectively), and the gamma chain and delta chain (Vγ and Vδ, respectively) of a native TCR generally have similar structures, with each domain comprising four conserved framework regions (FRs) and three CDRs. A single Vu or VP domain, or Vγ or Vδ domain, may be sufficient to confer binding specificity to a peptide-MHC complex.

Also provided herein are TCR fragments, including antigen-binding fragments. In some embodiments, the TCR is an antigen-binding portion thereof, such as a variant of a full-length TCR not containing the transmembrane and/or cytoplasmic region(s) thereof, which may be referred to as a full soluble TCR. In some embodiments, the TCR is a dimeric TCR (dTCR). In some embodiments, the TCR is a single-chain TCR (scTCR), such as a scTCR having a structure as described in PCT patent publication numbers WO2003/020763, WO2004/033685, or WO2011/044186. In certain embodiments, the TCR is a single-chain TCR fragment comprising an alpha chain variable region linked to a beta chain variable region, such as a scTv. In some embodiments, a scTv is also referred to as a scFv. A single-chain Tv or scTv refers in some aspects TCR fragments that comprise the variable alpha or gamma chain (Vα or Vγ) and variable beta or delta chain (Vβ or Vδ) domains of a TCR, wherein these domains are present in a single polypeptide chain. Generally, the Tv polypeptide further comprises a polypeptide linker between the Vα and Vβ domains or Vγ and Vδ domains which enables the scTv to form the desired structure for antigen binding. A diabody refers in some aspects to TCR fragments with two antigen-binding sites, which fragments comprise a Vu connected to a VP in the same polypeptide chain (Vα-Vβ) or a Vγ connected to a Vδ in the same polypeptide chain (Vγ-Vδ). By using a linker that is too short to allow pairing between the two domains on the same chain, the domains are forced to pair with the complementary domains of another chain and create two antigen-binding sites. Exemplary diabodies are described more fully in, for example, EP404097 and WO93111161. Fv refers in some aspects to a TCR fragment which contains a complete peptide-MHC complex recognition and peptide-MHC complex binding site. This region consists of a dimer of one TCRα chain and one TCRβ chain or one TCRγ chain and one TCRδ chain in tight, non-covalent association. It is in this configuration that the three CDRs of each variable domain interact to define a peptide-MHC complex binding site on the surface of the Vα-Vβ dimer or Vγ-Vδ dimer. Collectively, a combination of one or more of the CDRs from each of the Vα-Vγ chains or Vγ-Vδ chains confers peptide-MHC complex binding specificity to the TCR. For example, it would be understood that, for example, the CDRα3 and CDRβ3 or CDRγ3 and CDRδ3 could be sufficient to confer antigen-binding specificity to a TCR when transferred to Vu and VP chains or Vγ-Vδ chains of a recipient selected TCR or antigen-binding fragment thereof and this combination of CDRs can be tested for binding, affinity, etc. Furthermore, although the two domains of a Tv fragment (Vα and Vβ or Vγ and Vδ), are coded for by separate genes, they can be joined using recombinant methods by a synthetic linker that enables them to be made as a single protein chain in which the Vα and Vβ or Vγ and Vδ chain regions pair to form monovalent molecules (known as single chain Tv (scTv). Such scTvs are also intended to be encompassed within the peptide-MHC complex binding portion of a TCR.

A “bispecific TCR” refers in some aspects to a TCR that shows specificities to two different peptide-MHC complexes or two different types of peptide-MHC complexes. The terms as used herein specifically include, without limitation, TCRs which show binding specificity for a target peptide-MHC complex and to another peptide-MHC complex that facilitates delivery to a particular tissue. Similarly, multi-specific TCRs have two or more binding specificities. A linear TCR refers in some aspects to a pair of tandem Fd segments (e.g., Va-Cai-Va-Cai) which form a pair of antigen binding regions. Linear TCRs can be bispecific or monospecific.

An “antigen-binding domain” refers in some aspects to one or more fragments of a TCR that retain the ability to specifically bind to a peptide-MHC complex. Non-limiting examples of TCR fragments included within such terms include, but are not limited to, (i) a Tab fragment, a monovalent fragment consisting of the Vβ, Vα, C_{β} and Cα domains; (ii) a T(ab′)₂ fragment, a bivalent fragment containing two Tab fragments linked by a disulfide bridge at the hinge region; (iii) a Td fragment consisting of the Vα and Cα₁ domains; (iv) a Tv fragment containing the Vβ and Vα domains of a single arm of a TCR, including scTvs, (v) a dAb fragment (Ward et al., (1989) Nature 341:544 546), which contains a Vα domain; and (vi) an isolated CDR. Included in this definition are TCRs with a single alpha chain or a single beta chain.

Among the provided TCRs are humanized and human TCRs. A “humanized” TCR is a TCR in which all or substantially all CDR amino acid residues are derived from non-human CDRs and all or substantially all FR amino acid residues are derived from human FRs. A humanized TCR optionally may include at least a portion of a TCR constant region derived from a human TCR. A “humanized form” of a non-human TCR, refers to a variant of the non-human TCR that has undergone humanization, typically to reduce immunogenicity to humans, while retaining the specificity and affinity of the parental non-human TCR. In some embodiments, some FR residues in a humanized TCR are substituted with corresponding residues from a non-human TCR (e.g., the TCR from which the CDR residues are derived), e.g., to restore or improve TCR specificity or affinity. A “human TCR” is a TCR with an amino acid sequence corresponding to that of a TCR produced by a human or a human cell, or non-human source that utilizes human TCR repertoires or other human TCR-encoding sequences, including human TCR libraries. The term excludes humanized forms of non-human TCRs comprising non-human peptide-MHC complex binding regions, such as those in which all or substantially all CDRs are non-human. Human TCRs may be prepared by administering an immunogen to a transgenic animal that has been modified to produce intact human TCRs or intact TCRs with human variable regions in response to antigenic challenge. Such animals typically contain all or a portion of the human TCR loci, which replace the endogenous TCR loci, or which are present extrachromosomally or integrated randomly into the animal's chromosomes. In such transgenic animals, the endogenous TCR loci have generally been inactivated. Human TCRs also may be derived from human TCR libraries, including phage display and cell-free libraries, containing TCR-encoding sequences derived from a human repertoire.

The term “cancer neoantigen” or “neoantigen” or “neoepitope” can refer to antigens that are not encoded in a normal, non-mutated host genome. A neoantigen can relate to an antigen including one or more amino acid modifications compared to the parental antigen. For example, a neoantigen may be a tumor-associated neoantigen, wherein the term “tumor-associated neoantigen” can include a peptide or protein including amino acid modifications due to tumor-specific mutations. In some instances, a neoantigen represents either oncogenic viral proteins or abnormal proteins that arise as a consequence of somatic mutations. For example, a neoantigen can arise by the disruption of cellular mechanisms through the activity of viral proteins. Another example can be an exposure of a carcinogenic compound, which in some cases can lead to a somatic mutation. This somatic mutation can ultimately lead to the formation of a tumor/cancer. A neoantigen can be a class of tumor antigens which arise from tumor-specific changes in proteins. Neoantigens encompass, but are not limited to, tumor antigens which arise from, for example, a substitution in a protein sequence, a frame shift mutation, a fusion polypeptide, an in-frame deletion, an insertion, and expression of an endogenous retroviral polypeptide. A neoepitope can be an epitope that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope.

An “epitope” refers in some aspects to a portion of an antigen or other macromolecule capable of forming a binding interaction with the variable region binding pocket of a TCR. In some aspects, an epitope refers to a portion of a peptide-MHC complex capable of forming a binding interaction with the variable region binding pocket of a TCR. Such binding interactions can be manifested as an intermolecular contact with one or more amino acid residues of one or more CDRs. Peptide-MHC complex binding can involve, for example, a CDR3, a CDR3 pair, or in some instances, interactions of up to all six CDRs of the Vα and Vβ chains or Vγ or Vδ chains. An epitope can be a linear peptide sequence (e.g., “continuous”) or can be composed of noncontiguous amino acid sequences (e.g., “conformational” or “discontinuous”). A TCR can recognize one or more amino acid sequences. Therefore, an epitope can define more than one distinct amino acid sequence. In some aspects, a TCR can recognize one or more amino acid sequences or epitopes in the context of an MHC. Epitopes recognized by TCRs can be determined by peptide mapping and sequence analysis techniques well known to one of skill in the art. Binding interactions are manifested as intermolecular contacts with one or more amino acid residues of a CDR. An epitope can refer to an antigenic determinant in a molecule such as an antigen, e.g., to a part in or fragment of the molecule that is recognized by the immune system, for example, that is recognized by a T cell, in particular when presented in the context of MHC molecules. An epitope of a protein such as a tumor antigen can comprise a continuous or discontinuous portion of the protein and can be between 5 and 100, 5 and 50, 8 and 30, or 10 and 25 amino acids in length, for example, the epitope may be 9, 10, 11, 12, 13, 14, 15, 16, 17, 18, 19, 20 21, 22, 23, 24 or 25 amino acids in length.

The term “binding” refers to a direct association between two molecules, due to, for example, covalent, electrostatic, hydrophobic, and ionic and/or hydrogen-bond interactions under physiological conditions, and includes interactions such as salt bridges and water bridges, as well as any other conventional means of binding.

In some embodiments, reference to a TCR with “specific binding” refers to a situation in which a TCR will not show any significant binding to molecules other than the peptide-MHC complex containing the epitope recognized by the TCR. The term is also applicable where for example, an antigen binding domain is specific for a particular epitope which is carried by a number of peptide-MHC complexes, in which case the selected TCR or peptide-MHC complex binding fragment thereof carrying the peptide-MHC complex binding domain will be able to bind to the various peptide-MHC complexes carrying the epitope. The terms “preferentially binds” or “specifically binds” mean that the TCRs or fragments thereof bind to an epitope with greater affinity than it binds unrelated amino acid sequences, and, if cross-reactive to other polypeptides containing the epitope, are not toxic at the levels at which they are formulated for administration to human use. In one aspect, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the TCR or fragment thereof for unrelated amino acid sequences.

The term “affinity” refers to a measure of the strength of binding between two members of a binding pair (e.g., a human leukocyte antigen (HLA)-binding peptide and a class I or II HLA, or a peptide-HLA complex and a T cell receptor (TCR)). Affinity can be expressed as an equilibrium constant of the reversible binding of two agents and can be expressed as K_{D}, K_{A}, K_{off} or Kₒₙ. K_{D} refers to the dissociation constant between two members of a binding pair and has units of molarity. K_{A} refers to the affinity constant between two members of a binding pair is the inverse of the dissociation constant. Affinity may be determined experimentally, for example by surface plasmon resonance (SPR) using commercially available Biacore SPR units. K_{off} refers to the off-rate constant of two members of a binding pair, (e.g., the off-rate constant of an HLA-binding peptide and a class I or II HLA, or a peptide-HLA complex and a TCR). Kₒₙ refers to the on-rate constant of two members of a binding pair, (e.g., the on-rate constant of an HLA-binding peptide and a class I or II HLA, or a peptide-HLA complex and a TCR). Affinity of a binding protein to a ligand such as affinity of a TCR for an epitope can be, for example, from about 100 nanomolar (nM) to about 0.1 nM, from about 100 nM to about 1 picomolar (pM), or from about 100 nM to about 1 femtomolar (fM). The term “avidity” refers to the resistance of a complex of two or more agents to dissociation after dilution.

Throughout this disclosure, “binding data” results may be expressed in terms of an “IC₅₀.” Affinity may also be expressed as the inhibitory concentration 50 (IC₅₀), or the concentration at which 50% of a first member of a binding pair (e.g., a peptide) is displaced. Likewise, ln(IC₅₀) refers to the natural log of the IC₅₀. For example, an IC₅₀ may be the concentration of a tested peptide in a binding assay at which 50% inhibition of binding of a labeled reference peptide is observed. Given the conditions in which the assays are run (e.g., limiting HLA protein concentrations and/or labeled reference peptide concentrations), these values can approximate K_{D} values. Assays for determining binding are well known in the art and are described in detail, for example, in PCT publications WO 94/20127 and WO 94/03205, and other publications such as Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

The terms “major histocompatibility complex” and the abbreviation “MHC” can include MHC class I and MHC class II molecules and relate to a complex of genes which occurs in all vertebrates. MHC proteins or molecules can be important for signaling between lymphocytes and antigen presenting cells or diseased cells in immune reactions, wherein the MHC proteins or molecules bind peptides and present them for recognition by T cell receptors. The proteins encoded by the MHC can be expressed on the surface of cells, and display both self-antigens (peptide fragments from the cell itself) and non-self-antigens (e.g., fragments of invading microorganisms) to a T cell. The MHC region can be divided into three subgroups, class I, class II, and class III. MHC class I proteins can contain an α-chain and β2-microglobulin (not part of the MHC encoded by chromosome 15). They can present antigen fragments to cytotoxic T cells. MHC class II proteins can contain α- and β-chains and they can present antigen fragments to T-helper cells. MHC class III region can encode for other immune components, such as complement components and cytokines. The MHC can be both polygenic (there are several MHC class I and MHC class II genes) and polymorphic (there are multiple alleles of each gene).

The term “haplotype” can refer to the human leukocyte antigen (HLA) alleles found on one chromosome and the proteins encoded thereby. Haplotype may also refer to the allele present at any one locus within the MHC. Each class of MHC is represented by several loci: e.g., HLA-A (Human Leukocyte Antigen-A). HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-DQA1, HLA-DQB1, HLA-DPA1, HLA-DPB1, HLA-DPB2, HLA-DMA, HLA-DMB, HLA-DOA, and HLA-DOB for class II. The terms “HLA allele” and “MHC allele” are used interchangeably herein.

The terms “polynucleotide”, “nucleotide”, “nucleotide sequence”, “nucleic acid” and “oligonucleotide” are used interchangeably. They can refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. Polynucleotides can include nonstandard nucleotides, such as nucleotide analogs or modified nucleotides. In some embodiments, nonstandard nucleotides can stabilize hybrid formation. In some embodiments, nonstandard nucleotides can destabilize hybrid formation. In some embodiments, nonstandard nucleotides can enhance hybridization specificity. In some embodiments, nonstandard nucleotides can reduce hybridization specificity. Examples of nonstandard nucleotide modifications include 2′ O-Me, 2′ O-allyl, 2′ O-propargyl, 2′ O-alkyl, 2′ fluoro, 2′ arabino, 2′ xylo, 2′ fluoro arabino, phosphorothioate, phosphorodithioate, phosphoroamidates, 2′ Amino, 5-alkyl-substituted pyrimidine, 3′ deoxyguanosine, 5-halo-substituted pyrimidine, alkyl-substituted purine, halo-substituted purine, bicyclic nucleotides, 2′MOE, PNA molecules, LNA-molecules, LNA-like molecules, diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methyl guanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxy acetic acid methylester, uracil-5-oxy acetic acid (v), 5-methyl-2-thiouracil, 3-(3-amino-3-N-2-carboxypropyl) uracil, (acp3)w, 2,6-diaminopurine, and derivatives thereof. The sequence of nucleotides may be interrupted by non-nucleotide components. A polynucleotide may be further modified after polymerization, such as by conjugation with a labeling component.

“Complementarity” can refer to the ability of a nucleic acid to form hydrogen bond(s) with another nucleic acid sequence by either traditional Watson-Crick or other non-traditional types. A percent complementarity can indicate the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or can refer to two nucleic acids that hybridize under stringent conditions. Sequence identity, such as for the purpose of assessing percent complementarity, may be measured by any suitable alignment algorithm, including but not limited to the Needleman-Wunsch algorithm (e.g., the EMBOSS Needle aligner available at www.ebi.ac.uk/Tools/psa/emboss_needle/nucleotide.html, optionally with default settings), the BLAST algorithm (see e.g., the BLAST alignment tool available at blast.ncbi.nlm.nih.gov/Blast.cgi, optionally with default settings), or the Smith-Waterman algorithm. Optimal alignment may be assessed using any suitable parameters of a chosen algorithm, including default parameters.

The terms “polypeptide” and “protein” are used interchangeably to refer to a polymer of amino acid residues, and are not limited to a minimum length. For example, a polypeptide can comprise at least about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 peptides or amino acids. Examples of polypeptides include, but are not limited to, amino acid chains, proteins, peptides, hormones, polypeptide saccharides, lipids, glycolipids, phospholipids, antibodies, enzymes, kinases, receptors, transcription factors, and ligands. Polypeptides, including the provided TCRs and TCR chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, the twenty conventional amino acids and their abbreviations known to one skilled in the art follow conventional usage. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left hand direction is the amino terminal direction and the right hand direction is the carboxy-terminal direction, in accordance with standard usage and convention. Percent (%) sequence identity with respect to a reference polypeptide sequence (or nucleic acid sequence) is the percentage of amino acid residues (or nucleotides in case of nucleic acid sequence) in a candidate sequence that are identical with the amino acid residues (or nucleotides) in the reference polypeptide sequence (or nucleic acid sequence), after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

A “germline sequence” refers to a genetic sequence from the haploid gametes and those diploid cells from which they are formed. Germline DNA contains multiple gene segments that encode a single TCRα or TCRβ chain, or a single TCRγ or TCRδ chain. These gene segments are carried in the germ cells but cannot be transcribed and translated until they are arranged into functional genes. During T cell differentiation in the bone marrow, these gene segments are randomly shuffled by a dynamic genetic system capable of generating more than 10⁸ specificities.

Inhibition, “treatment” and “treating” are used interchangeably and refer to, for example, stasis of symptoms, prolongation of survival, partial or full amelioration of symptoms, and partial or full eradication of a condition, disease or disorder associated with excess levels of protein or correlated with protein activity. For example, treatment of cancer includes, but is not limited to, stasis, partial or total elimination of a cancerous growth or tumor. Treatment or partial elimination includes, for example, a fold reduction in growth or tumor size and/or volume such as about 2-fold, about 3-fold, about 4-fold, about 5-fold, about 10-fold, about 20-fold, about 50-fold, or any fold reduction in between. Similarly, treatment or partial elimination can include a percent reduction in growth or tumor size and/or volume of about 1%, 2%, 3%, 4%, 5%, 10%, 20%, 30%, 40%, 50%, 60%, 70%, 80%, 90%, 95% or any percentage reduction in between. Prevention refers to prophylaxis, prevention of onset of symptoms, prevention of progression of a disease or disorder associated with excess levels of protein or correlated with protein activity.

A “subject”, “individual”, “host” or “patient” refers to living organisms such as mammals. Examples of subjects and hosts include, but are not limited to, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks) fish (e.g., sharks) or frogs (e.g., Xenopus), and non-mammalian invertebrates, as well as transgenic species thereof. In certain aspects, a subject refers to a single organism (e.g., human). In certain aspects, or a group of individuals composing a small cohort having either a common immune factor to study and/or disease, and/or a cohort of individuals without the disease (e.g., negative/normal control) are provided. A subject from whom samples are obtained can either be inflicted with a disease and/or disorder (e.g., one or more allergies, infections, cancers or autoimmune disorders or the like) and can be compared against a negative control subject which is not affected by the disease.

A “kit” refers to a delivery system for delivering materials or reagents for carrying out a method disclosed herein. In some embodiments, kits include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains a plurality of primers. A packaging material refers to a physical structure housing the components of the kit. The packaging material can maintain the components sterilely and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method described herein.

The term “resistance mutation” refers to a mutation in a gene that allows the gene or the host cell containing the gene to become resistant to treatment with a drug. For example, BTK C481S mutation is a resistance mutation which can confer ibrutinib resistance.

### I. Overview

The present disclosure provides T cell receptors (TCRs) against neoantigens, isolated nucleic acid molecules encoding TCRs against neoantigens, T cells expressing said TCRs, and pharmaceutical compositions for use in the treatment of diseases involving malignant cells expressing said neoantigens.

Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a complementarity determining region 3 (CDR3) having at least 84% sequence identity to an amino acid sequence of SEQ ID NO: 3, and/or wherein the TCR beta chain construct comprises a complementarity determining region 3 (CDR3) having at least 84% sequence identity to an amino acid sequence selected from SEQ ID NO: 6. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 84% sequence identity to an amino acid sequence of SEQ ID NO: 17, and/or wherein the TCR beta chain construct comprises a complementarity determining region 3 (CDR3) having at least 84% sequence identity to an amino acid sequence selected from SEQ ID NO: 20. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 84% sequence identity to an amino acid sequence of SEQ ID NO: 31, and/or wherein the TCR beta chain construct comprises a complementarity determining region 3 (CDR3) having at least 84% sequence identity to an amino acid sequence selected from SEQ ID NO: 34. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 84% sequence identity to an amino acid sequence of selected from SEQ ID NOs: 3, 17, or 31. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR beta chain construct comprises a CDR3 having at least 84% sequence identity to an amino acid sequence of selected from SEQ ID NOs: 6, 20, or 34.

Provided herein is a nucleic acid encoding at least one T cell receptor (TCR) comprising a TCR alpha chain construct and/or TCR beta chain construct capable of specifically binding to an epitope from RAS in complex with a human MHC, wherein the CDR3 of the TCR does not comprise any one of SEQ ID NOs: 46-68. Provided herein is a nucleic acid encoding at least one T cell receptor (TCR) comprising a TCR alpha chain construct and/or TCR beta chain construct capable of specifically binding to an epitope from RAS in complex with a human MHC, wherein the epitope from RAS comprises a region having at least 70% sequence identity to an amino acid sequence selected from SEQ ID NOs: 43-45. In some embodiments, the epitope from RAS comprises a region having at least 70% sequence identity to an amino acid sequence selected from SEQ ID NOs: 43-44. In some embodiments, the epitope from RAS comprises a region having at least 70% sequence identity to an amino acid sequence selected from SEQ ID NO: 43. In some embodiments, the epitope from RAS comprises a region having at least 70% sequence identity to an amino acid sequence selected from SEQ ID NO: 44. In some embodiments, the epitope from RAS comprises a region having at least 70% sequence identity to an amino acid sequence selected from SEQ ID NO: 45.

Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a complementarity determining region 3 (CDR3) having at least 60%, 65%, 70%, 75%, 80%, 85%, 90%, 95%, 96%, 98%, 99% or 100% sequence identity to an amino acid sequence of SEQ ID NO: 77, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 80. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 60%, 65%, 70%, 75%, 80%, 85%, 90%, 95%, 96%, 98%, 99% or 100% sequence identity to an amino acid sequence of SEQ ID NO: 87, and/or wherein the TCR beta chain construct comprises a complementarity determining region 3 (CDR3) having at least 80% sequence identity to an amino acid sequence selected from SEQ ID NO: 90. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 60%, 65%, 70%, 75%, 80%, 85%, 90%, 95%, 96%, 98%, 99% or 100% sequence identity to an amino acid sequence of SEQ ID NO: 93, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 95. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 60%, 65%, 70%, 75%, 80%, 85%, 90%, 95%, 96%, 98%, 99% or 100% sequence identity to an amino acid sequence of SEQ ID NO: 100, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 101. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a complementarity determining region 3 (CDR3) having at least 60%, 65%, 70%, 75%, 80%, 85%, 90%, 95%, 96%, 98%, 99% or 100% sequence identity to an amino acid sequence of SEQ ID NO: 106, and/or wherein the TCR beta chain construct comprises a complementarity determining region 3 (CDR3) having at least 80% sequence identity to an amino acid sequence selected from SEQ ID NO: 109. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 60%, 65%, 70%, 75%, 80%, 85%, 90%, 95%, 96%, 98%, 99% or 100% sequence identity to an amino acid sequence of SEQ ID NO: 112, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 113. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 80% sequence identity to an amino acid sequence of SEQ ID NO: 116, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 117. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 80% sequence identity to an amino acid sequence of SEQ ID NO: 120, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 123. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a complementarity determining region 3 (CDR3) having at least 60%, 65%, 70%, 75%, 80%, 85%, 90%, 95%, 96%, 98%, 99% or 100% sequence identity to an amino acid sequence of SEQ ID NO: 126, and/or wherein the TCR beta chain construct comprises a complementarity determining region 3 (CDR3) having at least 60%, 65%, 70%, 75%, 80%, 85%, 90%, 95%, 96%, 98%, 99% or 100% sequence identity to an amino acid sequence selected from SEQ ID NO: 129. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a CDR3 having at least 60%, 65%, 70%, 75%, 80%, 85%, 90%, 95%, 96%, 98%, 99% or 100% sequence identity to an amino acid sequence of selected from SEQ ID NOs: 79, 87, 93, 100, 106, 112, 116, 120, or 126. Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR beta chain construct comprises a CDR3 having at least 60%, 65%, 70%, 75%, 80%, 85%, 90%, 95%, 96%, 98%, 99% or 100% sequence identity to an amino acid sequence of selected from SEQ ID NOs: 82, 90, 95, 101, 109, 113, 117, 123, or 129.

Provided herein is a nucleic acid encoding at least one T cell receptor (TCR) comprising a TCR alpha chain construct and/or TCR beta chain construct capable of specifically binding to an epitope from RAS in complex with a human MHC, wherein the epitope from RAS comprises a region having at least 60%, 65%, 70%, 75%, 80%, 85%, 90%, 95%, 96%, 98%, 99% or 100% sequence identity to an amino acid sequence selected from SEQ ID NOs: 132-135. In some embodiments, the epitope from RAS comprises a region having at least 60%, 65%, 70%, 75%, 80%, 85%, 90%, 95%, 96%, 98%, 99% or 100% sequence identity to an amino acid sequence selected from SEQ ID NOs: 132-135. In some embodiments, the epitope from RAS comprises a region having at least 70% sequence identity to an amino acid sequence selected from SEQ ID NO: 132. In some embodiments, the epitope from RAS comprises a region having at least 60%, 65%, 70%, 75%, 80%, 85%, 90%, 95%, 96%, 98%, 99% or 100% sequence identity to an amino acid sequence selected from SEQ ID NO: 133. In some embodiments, the epitope from RAS comprises a region having at least 60%, 65%, 70%, 75%, 80%, 85%, 90%, 95%, 96%, 98%, 99% or 100% sequence identity to an amino acid sequence selected from SEQ ID NO: 134. In some embodiments, the epitope from RAS comprises a region having at least 60%, 65%, 70%, 75%, 80%, 85%, 90%, 95%, 96%, 98%, 99% or 100% sequence identity to an amino acid sequence selected from SEQ ID NO: 135.

Provided herein is an isolated nucleic acid or a cell comprising a recombinant nucleic acid, wherein the nucleic acid encodes at least one T cell receptor (TCR) comprising a TCR alpha chain construct and/or a TCR beta chain construct, wherein the TCR specifically binds to an epitope from RAS in complex with a human MHC encoded by an HLA-A11:01 allele or an HLA-C01:02. In some embodiments, the TCR specifically binds to an epitope from RAS in complex with a human MHC encoded by an HLA-A11:01 allele. In some embodiments, the TCR specifically binds to an epitope from RAS in complex with a human MHC encoded by an HLA-C01:02.

Provided herein is an isolated nucleic acid or a cell comprising a recombinant nucleic acid, wherein the nucleic acid encodes at least one T cell receptor (TCR) comprising a TCR alpha chain construct and/or a TCR beta chain construct, wherein the TCR specifically binds to an epitope from RAS in complex with a human MHC encoded by an HLA-A68:01 allele, an HLA-C05:01, an HLA-C03:04, or an HLA-C03:03. In some embodiments, the TCR specifically binds to an epitope from RAS in complex with a human MHC encoded by an HLA-A68:01 allele. In some embodiments, the TCR specifically binds to an epitope from RAS in complex with a human MHC encoded by an HLA-C05:01. In some embodiments, the TCR specifically binds to an epitope from RAS in complex with a human MHC encoded by an HLA-C03:04. In some embodiments, the TCR specifically binds to an epitope from RAS in complex with a human MHC encoded by an HLA-C03:03.

In some embodiments, the TCR alpha chain construct comprises a variable region having at least 80% sequence identity an amino acid sequence selected from SEQ ID NO: 9. In some embodiments, the TCR beta chain construct comprises a variable region having at least 80% sequence identity to an amino acid sequence selected from SEQ ID NO: 12. In some embodiments, the TCR alpha chain construct and/or the TCR beta chain construct comprises a complementarity determining region 1 (CDR1) having at least 90% sequence identity to an amino acid sequence selected from SEQ ID NO: 1. In some embodiments, the TCR beta chain construct comprises a complementarity determining region 1 (CDR1) having at least 90% sequence identity to an amino acid sequence selected from SEQ ID NO: 4. In some embodiments, the TCR alpha chain construct comprises a complementarity determining region 2 (CDR2) having at least 90% sequence identity to an amino acid sequence selected from SEQ ID NO: 2. In some embodiments, the TCR beta chain construct comprises a complementarity determining region 2 (CDR2) having at least 90% sequence identity to an amino acid sequence selected from SEQ ID NO: 5. In some embodiments, the TCR alpha chain construct comprises a variable region having at least 80% sequence identity to SEQ ID NO: 9; and the TCR beta chain construct comprises a variable region having at least 80% sequence identity to SEQ ID NO: 12. In some embodiments, the TCR alpha chain construct comprises a CDR1 of SEQ ID NO: 1, a CDR2 of SEQ ID NO: 2, and a CDR3 of SEQ ID NO: 3; and the TCR beta chain construct comprises a CDR1 of SEQ ID NO: 4, a CDR2 of SEQ ID NO: 5, and a CDR3 of SEQ ID NO: 6.

In some embodiments, the TCR alpha chain construct comprises a variable region having at least 80% sequence identity an amino acid sequence selected from SEQ ID NO: 23. In some embodiments, the TCR beta chain construct comprises a variable region having at least 80% sequence identity to an amino acid sequence selected from SEQ ID NO: 26. In some embodiments, the TCR alpha chain construct and/or the TCR beta chain construct comprises a complementarity determining region 1 (CDR1) having at least 90% sequence identity to an amino acid sequence selected from SEQ ID NO: 15. In some embodiments, the TCR beta chain construct comprises a complementarity determining region 1 (CDR1) having at least 90% sequence identity to an amino acid sequence selected from SEQ ID NO: 18. In some embodiments, the TCR alpha chain construct comprises a complementarity determining region 2 (CDR2) having at least 90% sequence identity to an amino acid sequence selected from SEQ ID NO: 16. In some embodiments, the TCR beta chain construct comprises a complementarity determining region 2 (CDR2) having at least 90% sequence identity to an amino acid sequence selected from SEQ ID NO: 19. In some embodiments, the TCR alpha chain construct comprises a variable region having at least 80% sequence identity to SEQ ID NO: 23; and the TCR beta chain construct comprises a variable region having at least 80% sequence identity to SEQ ID NO: 26. In some embodiments, the TCR alpha chain construct comprises a CDR1 of SEQ ID NO: 15, a CDR2 of SEQ ID NO: 16, and a CDR3 of SEQ ID NO: 17; and the TCR beta chain construct comprises a CDR1 of SEQ ID NO: 18, a CDR2 of SEQ ID NO: 19, and a CDR3 of SEQ ID NO: 20.

In some embodiments, the TCR alpha chain construct comprises a variable region having at least 80% sequence identity an amino acid sequence selected from SEQ ID NO: 37. In some embodiments, the TCR beta chain construct comprises a variable region having at least 80% sequence identity to an amino acid sequence selected from SEQ ID NO: 40. In some embodiments, the TCR alpha chain construct and/or the TCR beta chain construct comprises a complementarity determining region 1 (CDR1) having at least 90% sequence identity to an amino acid sequence selected from SEQ ID NO: 29. In some embodiments, the TCR beta chain construct comprises a complementarity determining region 1 (CDR1) having at least 90% sequence identity to an amino acid sequence selected from SEQ ID NO: 32. In some embodiments, the TCR alpha chain construct comprises a complementarity determining region 2 (CDR2) having at least 90% sequence identity to an amino acid sequence selected from SEQ ID NO: 30. In some embodiments, the TCR beta chain construct comprises a complementarity determining region 2 (CDR2) having at least 90% sequence identity to an amino acid sequence selected from SEQ ID NO: 23. In some embodiments, the TCR alpha chain construct comprises a variable region having at least 80% sequence identity to SEQ ID NO: 37; and the TCR beta chain construct comprises a variable region having at least 80% sequence identity to SEQ ID NO: 40. In some embodiments, the TCR alpha chain construct comprises a CDR1 of SEQ ID NO: 29, a CDR2 of SEQ ID NO: 30, and a CDR3 of SEQ ID NO: 31; and the TCR beta chain construct comprises a CDR1 of SEQ ID NO: 32, a CDR2 of SEQ ID NO: 33, and a CDR3 of SEQ ID NO: 34.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 83. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 84. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 77. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 80. In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 78. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 81. In some embodiments, the TCR alpha chain construct comprises a CDR1 of SEQ ID NO: 77, a CDR2 of SEQ ID NO: 78, and a CDR3 of SEQ ID NO: 79; and the TCR beta chain construct comprises a CDR1 of SEQ ID NO: 80, a CDR2 of SEQ ID NO: 81, and a CDR3 of SEQ ID NO: 82.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 91. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 92. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 85. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 88.

In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 86. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 89. In some embodiments, the TCR alpha chain construct comprises a CDR1 of SEQ ID NO: 85, a CDR2 of SEQ ID NO: 86, and a CDR3 of SEQ ID NO: 87; and the TCR beta chain construct comprises a CDR1 of SEQ ID NO: 88, a CDR2 of SEQ ID NO: 89, and a CDR3 of SEQ ID NO: 90.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 96. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 97. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 15. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 94. In some embodiments, the TCR alpha chain construct comprises a CDR2 having at least 90% sequence identity to an amino acid sequence selected from SEQ ID NO: 16. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 81. In some embodiments, the TCR alpha chain construct comprises a CDR1 of SEQ ID NO: 15, a CDR2 of SEQ ID NO: 16, and a CDR3 of SEQ ID NO: 93; and the TCR beta chain construct comprises a CDR1 of SEQ ID NO: 94, a CDR2 of SEQ ID NO: 81, and a CDR3 of SEQ ID NO: 95.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 102. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 103. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 98. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 94. In some embodiments, the TCR alpha chain construct comprises a CDR2 having at least 90% sequence identity to an amino acid sequence selected from SEQ ID NO: 99. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 81. In some embodiments, the TCR alpha chain construct comprises a CDR1 of SEQ ID NO: 98, a CDR2 of SEQ ID NO: 99, and a CDR3 of SEQ ID NO: 100; and the TCR beta chain construct comprises a CDR1 of SEQ ID NO: 94, a CDR2 of SEQ ID NO: 81, and a CDR3 of SEQ ID NO: 101.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 110. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 111. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 104. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 107. In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 105. In some embodiments, the TCR beta chain construct comprises a CDR2 having at least 90% sequence identity to an amino acid sequence selected from SEQ ID NO: 108. In some embodiments, the TCR alpha chain construct comprises a CDR1 of SEQ ID NO: 104, a CDR2 of SEQ ID NO: 105, and a CDR3 of SEQ ID NO: 106; and the TCR beta chain construct comprises a CDR1 of SEQ ID NO: 107, a CDR2 of SEQ ID NO: 108, and a CDR3 of SEQ ID NO: 109.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 114. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 115. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 104. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 88. In some embodiments, the TCR alpha chain construct comprises a CDR2 having at least 90% sequence identity to an amino acid sequence selected from SEQ ID NO: 105. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 89. In some embodiments, the TCR alpha chain construct comprises a CDR1 of SEQ ID NO: 104, a CDR2 of SEQ ID NO: 105, and a CDR3 of SEQ ID NO: 112; and the TCR beta chain construct comprises a CDR1 of SEQ ID NO: 88, a CDR2 of SEQ ID NO: 89, and a CDR3 of SEQ ID NO: 113.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 118. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 119. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 104. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 107. In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 105. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 108. In some embodiments, the TCR alpha chain construct comprises a CDR1 of SEQ ID NO: 104, a CDR2 of SEQ ID NO: 105, and a CDR3 of SEQ ID NO: 116; and the TCR beta chain construct comprises a CDR1 of SEQ ID NO: 107, a CDR2 of SEQ ID NO: 108, and a CDR3 of SEQ ID NO: 117.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 124. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 125. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 104. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 121. In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 105. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 122. In some embodiments, the TCR alpha chain construct comprises a CDR1 of SEQ ID NO: 104, a CDR2 of SEQ ID NO: 105, and a CDR3 of SEQ ID NO: 120; and the TCR beta chain construct comprises a CDR1 of SEQ ID NO: 121, a CDR2 of SEQ ID NO: 122, and a CDR3 of SEQ ID NO: 123.

In some embodiments, the TCR alpha chain construct comprises a variable region having at least 60%, 65%, 70%, 75%, 80%, 85%, 90%, 95%, 96%, 98%, 99% or 100% sequence identity an amino acid sequence selected from SEQ ID NO: 130. In some embodiments, the TCR beta chain construct comprises a variable region having at least 60%, 65%, 70%, 75%, 80%, 85%, 90%, 95%, 96%, 98%, 99% or 100% sequence identity to an amino acid sequence selected from SEQ ID NO: 131. In some embodiments, the TCR alpha chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 29. In some embodiments, the TCR beta chain construct comprises a CDR1 having at least 60%, 65%, 70%, 75%, 80%, 85%, 90%, 95%, 96%, 98%, 99% or 100% sequence identity to an amino acid sequence selected from SEQ ID NO: 127. In some embodiments, the TCR alpha chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 30. In some embodiments, the TCR beta chain construct comprises a CDR2 having at least 60%, 65%, 70%, 75%, 80%, 85%, 90%, 95%, 96%, 98%, 99% or 100% sequence identity to an amino acid sequence selected from SEQ ID NO: 128. In some embodiments, the TCR alpha chain construct comprises a CDR1 of SEQ ID NO: 29, a CDR2 of SEQ ID NO: 30, and a CDR3 of SEQ ID NO: 126; and the TCR beta chain construct comprises a CDR1 of SEQ ID NO: 127, a CDR2 of SEQ ID NO: 128, and a CDR3 of SEQ ID NO: 129.

In yet another aspect, the present disclosure provides a host cell comprising the nucleic acid encoding a TCR against a neoantigen provided herein, a vector containing the nucleic acid sequence, or a protein encoded by the nucleic acid provided herein. In some embodiments, the host cell is a CD4⁺ T cell. In some embodiments, the host cell is a CD8⁺ T cell. The host cell may be a natural killer (NK) cell or a B cell. The host cell may be an immortalized cell line.

In yet another aspect, the present disclosure provides pharmaceutical compositions comprising the nucleic acid encoding a TCR against a neoantigen provided herein, a host cell comprising the nucleic acid encoding a TCR against a neoantigen provided herein, a vector containing the nucleic acid sequence, or a protein encoded by the nucleic acid provided herein. In some embodiments, provided herein also comprises a method of using the pharmaceutical compositions disclosed herein.

Also provided herein in an additional aspect is a method of treating a subject with a disease or condition, comprising administering to the subject a pharmaceutical composition disclosed herein. In some embodiments, the subject has cancer.

### II. T Cell Receptors (TCRs)

The ability of T cells to recognize antigens associated with various cancers or infectious organisms is conferred by its TCR, which is made up of both an alpha (α) chain and a beta (β) chain or a gamma (γ) and a delta (δ) chain. The proteins which make up these chains are encoded by DNA, which employs a unique mechanism for generating the tremendous diversity of the TCR. This multi-subunit immune recognition receptor associates with the CD3 complex and binds peptides presented by the MHC class I and II proteins on the surface of antigen-presenting cells (APCs). Binding of a TCR to the antigenic peptide on the APC is a central event in T cell activation, which occurs at an immunological synapse at the point of contact between the T cell and the APC.

Each TCR contains variable complementarity determining regions (CDRs), as well as framework regions (FRs) and a constant region. The constant region can comprise an a chain constant and a β chain constant. The α chain constant and the β chain constant can be derived from mouse. For example, mouse TCR α chain constant can be Uniprot accession P01849, and mouse β chain constant can be Uniprot accession P01852 or P01851. In some cases, the α chain constant and the β chain constant can be derived from human. For example, human TCR α chain constant can be Uniprot accession P01848, and human β chain constant can be Uniprot accession P01850 or A0A5B9. The amino acid sequence of the third complementarity-determining region (CDR3) loops of the α and β chain variable domains largely determines the sequence diversity of αβ T cells arising from recombination between variable (Vβ), diversity (Dβ), and joining (Jβ) gene segments in the β chain locus, and between analogous Vα and Jα gene segments in the α chain locus, respectively. The existence of multiple such gene segments in the TCR α and β chain loci allows for a large number of distinct CDR3 sequences to be encoded. Independent addition and deletion of nucleotides at the Vβ-Dβ, Dβ-Jβ, and Vα-Jα junctions during the process of TCR gene rearrangement further increases CDR3 sequence diversity. In this respect, immunocompetence is reflected in the diversity of TCRs. The γδ TCR is distinctive from the ap TCR in that it encodes a receptor that interacts closely with the innate immune system. TCRγδ, is expressed early in development, has specialized anatomical distribution, has unique pathogen and small-molecule specificities, and has a broad spectrum of innate and adaptive cellular interactions. Early in ontogeny, as the restricted subsets of TCRγδ cells populate various tissues prenatally, a biased pattern of TCRγ V and J segment expression is established.

The TCRs provided herein target may be engineered TCRs, for example, chimeric antigen receptors (CARs). CARs can be composed of three regions: an ectodomain, a transmembrane domain and an endodomain.

An ectodomain can be the region of the receptor that is exposed to the extracellular fluid and can consist of an antigen recognition region. In some embodiments, an ectodomain further comprises and a spacer. In some embodiments, an ectodomain further comprises and a signal peptide. A signal peptide can direct the nascent protein into the endoplasmic reticulum. A signal protein in a CAR may be a single-chain variable fragment (scFv). A fusion protein may be a protein that is formed by merging two or more genes that code originally for different proteins but when they are translated in the cell, the translation produces one or more polypeptides with functional properties derived for each of the original genes. A scFv is a chimeric protein made up of a light chain domain and heavy chain variable domain connected with a short linker peptide. The linker may comprise hydrophilic residues with stretches of glycine and/or serine residues. The linker may comprise stretches of glutamate and lysine residues, which can improve solubility.

A transmembrane domain can be a hydrophobic domain that spans the membrane. In some embodiments, a transmembrane domain comprises an alpha-helical domain. A transmembrane domain may be functional for the stability of the receptor as a whole. In some embodiments, a transmembrane domain comprises a transmembrane domain from the most membrane proximal component of an endodomain. In some embodiments, a transmembrane domain comprises a CD3-zeta transmembrane domain. In some embodiments, a transmembrane domain allows for incorporation of an artificial TCR into a native TCR complex. In some embodiments, a transmembrane domain comprises a CD28 transmembrane domain.

An endodomain can be a functional intracellular portion of a receptor, such as a TCR or CAR. After antigen recognition, receptors cluster and a signal may be transmitted to the cell. In some embodiments, an endodomain comprises aCD3-zeta intracellular domain. In some embodiments, an endodomain comprises at least one ITAM. In some embodiments, an endodomain comprises at least 3 or at least 3 ITAMs. In some embodiments, an endodomain comprises a CD28 intracellular domain. In some embodiments, an endodomain comprises an OX40 intracellular domain. In some embodiments, an endodomain comprises a chimeric intracellular domain. For example, an endodomain can comprises aCD28 intracellular domain, an OX40 intracellular domain and a CD3-zeta intracellular domain.

### IV. T Cells

T cells belong to a group of white blood cells known as lymphocytes, and play a central role in cell-mediated immunity. T cells include CD4⁺ T cells (helper T cells) and CD8⁺ T cells (cytotoxic T cells). CD4⁺ T cells can assist other white blood cells in immunologic processes, including maturation of B-cells and activation of cytotoxic T cells and macrophages. CD4⁺ T cells are activated when presented with peptide antigens by MHC class II molecules expressed on the surface of antigen presenting cells (APCs). Once activated, the T cells can divide rapidly and secrete cytokines that regulate the active immune response. CD8⁺ T cells can destroy virally infected cells and tumor cells, and can also be implicated in transplant rejection. CD8⁺ T cells can recognize their targets by binding to antigen associated with MHC class I, which is present on the surface of nearly every cell of the body. Most T cells have a T cell receptor (TCR). The ability of T cells to recognize antigens associated with various cancers or infectious organisms is conferred by its TCR, which is made up of both an alpha (α) chain and a beta (β) chain or a gamma (γ) and a delta (δ) chain. The proteins which make up these chains are encoded by DNA, which employs a unique mechanism for generating the diversity of the TCR. This multi-subunit immune recognition receptor can associate with the CD3 complex and bind peptides presented by the MHC class I and II proteins on the surface of antigen-presenting cells (APCs). The first signal in activation of T cells can be provided by binding of the T cell receptor to a short peptide presented by the MHC on another cell. This ensures that only a T cell with a TCR specific to that peptide is activated. The partner cell is usually an antigen presenting cell such as a professional antigen presenting cell, usually a dendritic cell in the case of naive responses, although B-cells and macrophages can be important APCs. Binding of a TCR to the antigenic peptide on the APC can be a central event in T cell activation, which occurs at an immunological synapse at the point of contact between the T cell and the APC.

T cells can be prepared according to methods known in the art. T cells can be an enriched T cell preparation, an APC-depleted cell preparation, or a substantially purified T cell preparation. T cells can be a mixed T cell population or a purified T cell subset. T cells can be an enriched T cell preparation containing a number or percentage of T cells that is increased with respect to an isolated population of T cells.

T cells, or a subset of T cells, can be obtained from various lymphoid tissues. T cells can be obtained from a number of sources, including peripheral blood mononuclear cells (PBMCs), bone marrow, thymus, tissue biopsy, tumor, lymph node tissue, gut associated lymphoid tissue, mucosa associated lymphoid tissue, spleen tissue, lymphoid tissue, and tumors. The term “peripheral blood lymphocytes” (PBL) and its grammatical equivalents as used herein can refer to lymphocytes that circulate in the blood (e.g., peripheral blood). Peripheral blood lymphocytes can refer to lymphocytes that are not localized to organs. Peripheral blood lymphocytes can comprise T cells, NK cells, B cell, or any combinations thereof.

The method can comprise isolating T cells from a subject. The method can comprise obtaining T cells isolated from a subject. T cells can be obtained from T cell lines. T cells can be obtained from autologous sources. T cells can be obtained from allogeneic sources. T cells may also be obtained from a xenogeneic source, for example, from mouse, rat, non-human primate, and pig.

T cells can be an APC-depleted cell preparation. T cells can be substantially free of APCs. For example, T cells can comprise T cells separated from over 75% of APCs. In an exemplary embodiment, peripheral blood mononuclear cells (PBMCs) can be obtained from blood, e.g., in heparinized vials. PBMCs can be separated from red blood cells by centrifugation and PBMCs recovered from the interface. The recovered PBMCs optionally can be washed (e.g., with PBS).

T cell purification can be achieved, for example, by positive or negative selection including, but not limited to, the use of antibodies directed to CD2, CD3, CD4, CD5, CD8, CD14, CD19, and/or MHC class II molecules. A specific T cell subset, such as CD28⁺, CD4⁺, CD8⁺, CD45RA⁺, and/or CD45RO⁺ T cells, can be isolated by positive or negative selection techniques. For example, CD3⁺, CD28⁺ T cells can be positively selected using CD3/CD28 conjugated magnetic beads. In one aspect of the present invention, enrichment of a T cell population by negative selection can be accomplished with a combination of antibodies directed to surface markers unique to the negatively selected cells.

For example, a T cell sample can comprise cells from a subject's circulating blood and can be obtained by apheresis or leukapheresis. A T cell sample may contain lymphocytes, including T cells, monocytes, granulocytes, B cells, other nucleated white blood cells, red blood cells, and/or platelets. Undesirable components of the T cell sample can be removed and the remaining T cells can be suspended in culture media. For example, cells can be washed to remove the plasma fraction. For example, T cells can be isolated from peripheral blood lymphocytes by lysing the red blood cells and by centrifugation through a PERCOLL™ gradient.

In some embodiments, a T cell comprises at least one T cell receptor (TCR) comprising a TCR alpha chain construct and/or TCR beta chain construct capable of specifically binding to an epitope from RAS in complex with a human MHC. In some embodiments, a T cell comprises at least one T cell receptor (TCR) comprising a TCR alpha chain construct and/or TCR beta chain construct capable of specifically binding to an epitope from TMPRSS2:ERG in complex with a human MHC. In some embodiments, a T cell comprises at least one T cell receptor (TCR) comprising a TCR alpha chain construct and/or TCR beta chain construct capable of specifically binding to an epitope from GATA3 in complex with a human MHC. In some embodiments, a host cell comprises at least one TCR disclosed herein, wherein the host cell is a CD4+ T cell. In some embodiments, the host cell is a CD8+ T cell. In some embodiments, the host cell is an autologous cell. In some embodiments, the host cell is an allogeneic cell. In some embodiments, the host cell is a human cell. In some other cases, a host cell may be natural killer (NK) cell, a B cell, or an immortalized cell line.

In some embodiments, T cells can be obtained by positive selection and/or negative selection. In positive selection, an affinity agent (such as an antibody, an antibody fragment, and aptamer) can be used to bind a cell surface marker expressed on the population of cells, for example, CD3 for T cells. Using the affinity agent, the T cells can be labeled. The labeled T cells can then be enriched using various methods that are well-known in the art. Non-limiting examples of those methods include fluorescent-activated cell sorting (FACS) and (para)magnetic particle-based cell separation (e.g., MACS cell separation kits from Miltenyi Biotec).

In negative selection, affinity agents can be used to bind cell surface markers expressed on blood cells other than the desired population. For example, when attempting to isolate T cells, a cocktail of affinity agents can be used to label B cells, NK cells, monocytes, platelets, dendritic cells, granulocytes, and erythrocytes. The labeled cells can then be depleted, leaving the T cells enriched. The exemplary methods to deplete labeled cells include FACS and (para)magnetic particle-based cell separation.

In addition to labeling-based isolation, special growth condition may be used to promote the growth of one particular cell population. For example, the special growth condition can be obtained using special cytokines or growth factors. For another example, in culture media containing phytohemagglutinin (PHA), IL-2, and/or IL-15, T cells may preferentially proliferate.

In some situations, non-functional markers may be used to isolate T cells since binding of functional markers such as binding of CD3 by anti-CD3 antibody (alone or conjugated to magnetic particles) may trigger unwanted signaling events on T cells. Therefore, a cocktail of antibodies against CD14, CD15, CD16, CD19, CD34, CD36, CD56, CD123, and CD235a (glycophorin A) can be used to isolate T cells.

Detailed protocol can be found in published literatures (e.g., see Lefort et al., J Vis Exp. 2010; (40): 2017) which are incorporated by reference herein. T cell isolation kits can be obtained from, for example, STEMCELL Technologies, Thermofisher, and Miltenyi Biotec.

The T cell described herein may be an allogeneic T cell.

In some embodiments, the T cell may be a genetically-modified cell comprising in its genome a modified human T cell receptor (TCR) alpha chain gene and/or a modified human TCR beta chain gene, wherein the cell has reduced cell-surface expression of the endogenous TCR.

Gene-editing nucleases may be employed in order to disrupt components of the TCR. The TCR alpha chain (TCRα) is encoded by a single TRAC gene and pairs with the TCR beta chain (TCRβ) encoded by two TCRB genes. Since the TCR α/β dimer can produce a fully functioning TCR complex, disrupting TCRα and/or TCRβ function may reduce (even eliminate) endogenous TCR expression.

Various methods may be used to disrupt endogenous TCRα or TCRβ genes. For example, four classes of gene editing proteins exist that share a common mode of action in binding a user defined sequence of DNA and mediating a double stranded DNA break (DSB). Zinc finger nucleases (ZFN) are heterodimeric arrays that co-localize at a target DNA site. ZFNs include individual finger subunits that bind DNA and are tethered to the Fokl nuclease domain that cleaves DNA. Transcription activator-like effector nucleases (TALEN) include repeating units that bind DNA by virtue of a hypervariable two amino acid sequence (repeat variable diresidue; RVD) that governs DNA base recognition. Similar to ZFNS, TALENs function as dimeric proteins that are fused to the Fokl endonuclease domain for DSB generation. Meganucleases (MN) are monomeric proteins with innate nuclease activity that are derived from bacterial homing endonucleases and engineered for a unique target site. The clustered regularly interspaced short palindromic repeats (CRISPR) and associated Cas9 nuclease platform involves a small guide RNA (gRNA) transcript that contacts a target DNA sequence via Watson-Crick base pairing and the Cas9 nuclease that cleaves the DNA.

In some embodiments, introducing the genome-editing nuclease into the T cell includes introducing into the T cell a polynucleotide that encodes the genome-editing nuclease.

In some embodiments, introducing the genome-editing nuclease into the T cell includes introducing into the T cell a Cas9 polypeptide. In some embodiments, the genome-editing nuclease includes a TALEN nuclease, a CRISPR/Cas9 nuclease, or a megaTAL nuclease.

In some embodiments, the CRISPR/Cas9 nuclease is derived from either Streptococcus pyogenes or Staphylococcus aureus. In some of these embodiments, the CRISPR/Cas9 nuclease includes a nuclease-resistant gRNA such as, for example, at least one 2′-OMe-phosphorothioate modified base, at least one 2′-O-methyl modified base, or at least one 2′-O-methyl 3′ thioPACE modified base.

In some embodiments, the TALEN nuclease or the megaTAL nuclease is encoded by an RNA that has an exogenous polyadenylation signal.

In some embodiments, the method described herein may further include culturing the T cell under conditions effective for expanding the population of genome-modified T cells.

In some embodiments, disrupting expression of TCRα and/or TCRβ further disrupts assembly of TCRα and TCRβ. In some embodiments, disrupting expression of TCRα further disrupts formation of a complex between TCR and CD3. In some embodiments, disrupting expression of TCRα involves further disrupting assembly of TCRα and TCRβ.

In some embodiments, a genetically-modified T cell comprises a disrupted TCR alpha chain and/or beta chain and an inactivated gene encoding immune checkpoint protein such as PD1 and CTLA-4. This may be made possible by gene inactivation using specific TALE-nucleases directed against TCRalpha or TCRbeta coupled with inactivation of genes encoding immune checkpoint protein such as PD1 and CTLA-4.

In some embodiments, the genetic modification relies on the inactivation of one gene, or two genes selected from the group consisting of PD1, CTLA-4, LAG3, Tim3, BTLA, BY55, TIGIT, B7H5, LAIR1, SIGLEC10, 2B4, TCR alpha and TCR beta. In some embodiments, the genetic modification relies on the inactivation of two genes selected from the group consisting of PD1 and TCR alpha, PD1 and TCR beta, CTLA-4 and TCR alpha, CTLA-4 and TCR beta, LAG 3 and TCR alpha, LAG 3 and TCR beta, Tim3 and TCR alpha, Tim3 and TCR beta, BTLA and TCR alpha, BTLA and TCR beta, BY55 and TCR alpha, BY55 and TCR beta, TIGIT and TCR alpha, TIGIT and TCR beta, B7H5 and TCR alpha, B7H5 and TCR beta, LAIR1 and TCR alpha, LAIR1 and TCR beta, SIGLEC10 and TCR alpha, SIGLEC10 and TCR beta, 2B4 and TCR alpha, 2B4 and TCR beta. In some embodiments, the genetic modification relies on the inactivation of more than two genes. The genetic modification may be operated ex-vivo.

### V. TCRs Specific to RAS Peptide-MHC Complexes

Provided herein is a nucleic acid encoding at least one T cell receptor (TCR) comprising a TCR alpha chain construct and/or TCR beta chain construct capable of specifically binding to an epitope from RAS in complex with a human MHC, wherein the TCR alpha chain construct comprises a complementarity determining region 3 (CDR3) having at least 84% sequence identity to an amino acid sequence selected from SEQ ID NO: 3 and/or wherein the TCR beta chain construct comprises a complementarity determining region 3 (CDR3) having at least 84% sequence identity to an amino acid sequence selected from SEQ ID NO: 6. Provided herein is a nucleic acid encoding at least one TCR comprising a TCR alpha chain construct and/or TCR beta chain construct capable of specifically binding to an epitope from RAS in complex with a human MHC, wherein the TCR alpha chain construct comprises a CDR3 having at least 84% sequence identity to an amino acid sequence selected from SEQ ID NO: 17 and/or wherein the TCR beta chain construct comprises a CDR3 having at least 84% sequence identity to an amino acid sequence selected from SEQ ID NO: 20. Provided herein is a nucleic acid encoding at least one TCR comprising a TCR alpha chain construct and/or TCR beta chain construct capable of specifically binding to an epitope from RAS in complex with a human MHC, wherein the TCR alpha chain construct comprises a CDR3 having at least 84% sequence identity to an amino acid sequence selected from SEQ ID NO: 31 and/or wherein the TCR beta chain construct comprises a CDR3 having at least 84% sequence identity to an amino acid sequence selected from SEQ ID NO: 34.

Provided herein is a nucleic acid encoding at least one T cell receptor (TCR) comprising a TCR alpha chain construct and/or TCR beta chain construct capable of specifically binding to an epitope from RAS in complex with a human MHC, wherein the epitope from RAS comprises a region having at least 70% sequence identity to an amino acid sequence selected from SEQ ID NOs: 43-45. In some embodiments, the epitope from RAS comprises a region having at least 70% sequence identity to an amino acid sequence selected from SEQ ID NOs: 43-44. In some embodiments, the epitope from RAS comprises a region having at least 70% sequence identity to an amino acid sequence selected from SEQ ID NO: 43. In some embodiments, the epitope from RAS comprises a region having at least 70% sequence identity to an amino acid sequence selected from SEQ ID NO: 44. In some embodiments, the epitope from RAS comprises a region having at least 70% sequence identity to an amino acid sequence selected from SEQ ID NO: 45.

Provided herein is an isolated nucleic acid or a cell comprising a recombinant nucleic acid, wherein the nucleic acid encodes at least one T cell receptor (TCR) comprising a TCR alpha chain construct and/or a TCR beta chain construct; wherein the TCR specifically binds to an epitope from RAS in complex with a human MHC encoded by an HLA-A11:01 allele. In some embodiments, the TCR alpha chain construct comprises a variable region having at least 80% sequence identity to an amino acid sequence selected from SEQ ID NO: 9, wherein the TCR specifically binds to an epitope from RAS in complex with a human MHC encoded by an HLA-A11:01 allele. In some embodiments, the TCR beta chain construct comprises a variable region having at least 80% sequence identity to an amino acid sequence selected from SEQ ID NO: 12, wherein the TCR specifically binds to an epitope from RAS in complex with a human MHC encoded by an HLA-A11:01 allele. Provided herein is an isolated nucleic acid or a cell comprising a recombinant nucleic acid, wherein the nucleic acid encodes at least one TCR comprising a TCR alpha chain construct and/or a TCR beta chain construct; wherein the TCR specifically binds to an epitope from RAS in complex with a human MHC encoded by an HLA-C01:02 allele. In some embodiments, the TCR alpha chain construct comprises a variable region having at least 80% sequence identity to an amino acid sequence selected from SEQ ID NO: 23, wherein the TCR specifically binds to an epitope from RAS in complex with a human MHC encoded by an HLA-C01:02 allele. In some embodiments, the TCR beta chain construct comprises a variable region having at least 80% sequence identity to an amino acid sequence selected from SEQ ID NO: 26, wherein the TCR specifically binds to an epitope from RAS in complex with a human MHC encoded by an HLA-C01:02 allele. Provided herein is an isolated nucleic acid or a cell comprising a recombinant nucleic acid, wherein the nucleic acid encodes at least one TCR comprising a TCR alpha chain construct and/or a TCR beta chain construct; wherein the TCR specifically binds to an epitope from RAS in complex with a human MHC encoded by an HLA-C01:02 allele. In some embodiments, the TCR alpha chain construct comprises a variable region having at least 80% sequence identity to an amino acid sequence selected from SEQ ID NO: 37, wherein the TCR specifically binds to an epitope from RAS in complex with a human MHC encoded by an HLA-C01:02 allele. In some embodiments, the TCR beta chain construct comprises a variable region having at least 80% sequence identity to an amino acid sequence selected from SEQ ID NO: 40, wherein the TCR specifically binds to an epitope from RAS in complex with a human MHC encoded by an HLA-C01:02 allele.

In some embodiments, the TCR alpha chain construct as described above comprises a complementarity determining region 1 (CDR1) having at least 90% sequence identity to an amino acid sequence selected from SEQ ID NO: 1. In some embodiments, the TCR beta chain construct as described above comprises a complementarity determining region 1 (CDR1) having at least 90% sequence identity to an amino acid sequence selected from SEQ ID NO: 4. In some embodiments, the TCR alpha chain construct comprises a complementarity determining region 2 (CDR2) having at least 90% sequence identity to an amino acid sequence selected from SEQ ID NO: 2. In some embodiments, the TCR beta chain construct comprises a complementarity determining region 2 (CDR2) having at least 90% sequence identity to an amino acid sequence selected from SEQ ID NO: 5. In some embodiments, the TCR alpha chain construct comprises a complementarity determining region 3 (CDR3) having at least 90% sequence identity to an amino acid sequence selected from SEQ ID NO: 3. In some embodiments, the TCR beta chain construct comprises a complementarity determining region 3 (CDR3) having at least 90% sequence identity to an amino acid sequence selected from SEQ ID NO: 6. In some embodiments, the TCR alpha chain construct comprises a variable region having at least 80% sequence identity to SEQ ID NO: 9; and the TCR beta chain construct comprises a variable region having at least 80% sequence identity to SEQ ID NO: 12. In some embodiments, the TCR alpha chain construct comprises a CDR1 of SEQ ID NO: 1, a CDR2 of SEQ ID NO: 2, and a CDR3 of SEQ ID NO: 3; and the TCR beta chain construct comprises a CDR1 of SEQ ID NO: 4, a CDR2 of SEQ ID NO: 5, and a CDR3 of SEQ ID NO: 6. In some embodiments, the TCR alpha chain construct as described above comprises a CDR1 having at least 90% sequence identity to an amino acid sequence selected from SEQ ID NO: 15. In some embodiments, the TCR beta chain construct as described above comprises a CDR1 having at least 90% sequence identity to an amino acid sequence selected from SEQ ID NO: 18. In some embodiments, the TCR alpha chain construct comprises a CDR2 having at least 90% sequence identity to an amino acid sequence selected from SEQ ID NO: 16. In some embodiments, the TCR beta chain construct comprises a CDR2 having at least 90% sequence identity to an amino acid sequence selected from SEQ ID NO: 19. In some embodiments, the TCR alpha chain construct comprises a CDR3 having at least 90% sequence identity to an amino acid sequence selected from SEQ ID NO: 17. In some embodiments, the TCR beta chain construct comprises a CDR3 having at least 90% sequence identity to an amino acid sequence selected from SEQ ID NO: 20. In some embodiments, the TCR alpha chain construct comprises a variable region having at least 80% sequence identity to SEQ ID NO: 23; and the TCR beta chain construct comprises a variable region having at least 80% sequence identity to SEQ ID NO: 26. In some embodiments, the TCR alpha chain construct comprises a CDR1 of SEQ ID NO: 15, a CDR2 of SEQ ID NO: 16, and a CDR3 of SEQ ID NO: 17; and the TCR beta chain construct comprises a CDR1 of SEQ ID NO: 18, a CDR2 of SEQ ID NO: 19, and a CDR3 of SEQ ID NO: 20. In some embodiments, the TCR alpha chain construct as described above comprises a CDR1 having at least 90% sequence identity to an amino acid sequence selected from SEQ ID NO: 29. In some embodiments, the TCR beta chain construct as described above comprises a CDR1 having at least 90% sequence identity to an amino acid sequence selected from SEQ ID NO: 32. In some embodiments, the TCR alpha chain construct comprises a CDR2 having at least 90% sequence identity to an amino acid sequence selected from SEQ ID NO: 30. In some embodiments, the TCR beta chain construct comprises a CDR2 having at least 90% sequence identity to an amino acid sequence selected from SEQ ID NO: 33. In some embodiments, the TCR alpha chain construct comprises a CDR3 having at least 90% sequence identity to an amino acid sequence selected from SEQ ID NO: 31. In some embodiments, the TCR beta chain construct comprises a CDR3 having at least 90% sequence identity to an amino acid sequence selected from SEQ ID NO: 34. In some embodiments, the TCR alpha chain construct comprises a variable region having at least 80% sequence identity to SEQ ID NO: 37; and the TCR beta chain construct comprises a variable region having at least 80% sequence identity to SEQ ID NO: 40. In some embodiments, the TCR alpha chain construct comprises a CDR1 of SEQ ID NO: 29, a CDR2 of SEQ ID NO: 30, and a CDR3 of SEQ ID NO: 31; and the TCR beta chain construct comprises a CDR1 of SEQ ID NO: 32, a CDR2 of SEQ ID NO: 33, and a CDR3 of SEQ ID NO: 34.

Provided herein is a recombinant nucleic acid encoding a T cell receptor (TCR) comprising a TCR beta chain construct and a TCR alpha chain construct, wherein the TCR can specifically bind to a mutated epitope from human RAS in complex with a human MHC encoded by an HLA-A68:01 allele. The mutated epitope from human RAS can be characterized by a GT2V mutation. In some embodiments, the TCR beta chain construct comprises a complementarity determining region 3 (CDR3), wherein the CDR3 has an amino acid sequence of SEQ ID NO: 82. In some embodiments, the TCR beta chain construct comprises a complementarity determining region 1 (CDR1), wherein the CDR1 has an amino acid sequence of SEQ ID NO: 80. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 84. In some embodiments, the TCR beta chain construct comprises a variable region having at least 90% sequence identity to an amino acid sequence set forth in SEQ ID NO: 84. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 84. In some embodiments, the TCR beta chain construct comprises a variable region having an amino acid sequence set forth in SEQ ID NO: 84. In some embodiments, the TCR further comprises a TCR alpha chain construct having a CDR1, a CDR2, and a CDR3, wherein, the CDR1 has an amino acid sequence set forth in SEQ ID NO: 77; the CDR2 has an amino acid sequence set forth in SEQ ID NO: 78; and, the CDR3 has an amino acid sequence set forth in SEQ ID NO: 79. In some embodiments, the TCR alpha chain construct comprises a variable region having at least 80% sequence identity to an amino acid sequence set forth in SEQ ID NO: 83. In some embodiments, the TCR alpha chain construct comprises a variable region having at least 90% sequence identity to an amino acid sequence set forth in SEQ ID NO: 83. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 83. In some embodiments, the TCR alpha chain construct comprises a variable region having an amino acid sequence set forth in SEQ ID NO: 83. In some embodiments, the epitope from human RAS comprising the mutation G12V is at least 60%, 65%, 70%, 75%, 80%, 85%, 90%, 95%, 96%, 98%, 99% or 100% sequence identical to SEQ ID NO: 43 or 44.

Provided herein is a recombinant nucleic acid encoding a T cell receptor (TCR) comprising a TCR beta chain construct and a TCR alpha chain construct, wherein the TCR can specifically bind to a mutated epitope from human RAS in complex with a human MHC encoded by an HLA-C03:03 allele. The mutated epitope from human RAS can be characterized by a G12V mutation. Provided herein is a recombinant nucleic acid encoding a T cell receptor (TCR) comprising a TCR beta chain construct and a TCR alpha chain construct, wherein the TCR can specifically bind to a mutated epitope from human RAS in complex with a human MHC encoded by an HLA-C03:04 allele. The mutated epitope from human RAS can be characterized by a G12V mutation. Provided herein is a recombinant nucleic acid encoding a T cell receptor (TCR) comprising a TCR beta chain construct and a TCR alpha chain construct, wherein the TCR can specifically bind to a mutated epitope from human RAS in complex with a human MHC encoded by an HLA-C03:03 allele and a human MHC encoded by an HLA-C03:04 allele. The mutated epitope from human RAS can be characterized by a G12V mutation. In some embodiments, the TCR binds to (i) the mutated epitope from human RAS in complex with the human MHC encoded by the HLA-C03:03 allele with a K_{D} of at most 500 nM, at most 250 nM, at most 50 nM, at most 25 nM, at most 10 nM, or at most 5 nM; and (ii) the mutated epitope from human RAS in complex with the human MHC encoded by the HLA-C03:04 allele with a K_{D} of at most 500 nM, at most 250 nM, at most 50 nM, at most 25 nM, at most 10 nM, or at most 5 nM. In some embodiments, the TCR beta chain construct comprises a complementarity determining region 3 (CDR3), wherein the CDR3 has an amino acid sequence of SEQ ID NO: 109. In some embodiments, the TCR beta chain construct comprises a complementarity determining region 1 (CDR1), wherein the CDR1 has an amino acid sequence of SEQ ID NO: 107. In some embodiments, the TCR beta chain construct comprises a complementarity determining region 2 (CDR2), wherein the CDR2 has an amino acid sequence of SEQ ID NO: 108. In some embodiments, the TCR beta chain construct comprises a variable region having at least 80% sequence identity to an amino acid sequence set forth in SEQ ID NO: 111. In some embodiments, the TCR beta chain construct comprises a variable region having at least 90% sequence identity to an amino acid sequence set forth in SEQ ID NO: 111. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 111. In some embodiments, the TCR beta chain construct comprises a variable region having an amino acid sequence set forth in SEQ ID NO: 111. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 106. In some embodiments, the TCR alpha chain construct comprises a variable region having at least 80% sequence identity to an amino acid sequence set forth in SEQ ID NO: 110. In some embodiments, the TCR alpha chain construct comprises a variable region having at least 90% sequence identity to an amino acid sequence set forth in SEQ ID NO: 110. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 110. In some embodiments, the TCR alpha chain construct comprises a variable region having an amino acid sequence set forth in SEQ ID NO: 110.

In some embodiments, the TCR beta chain construct comprises a complementarity determining region 3 (CDR3), wherein the CDR3 has an amino acid sequence of SEQ ID NO: 113. In some embodiments, the TCR beta chain construct comprises a complementarity determining region 1 (CDR1), wherein the CDR1 has an amino acid sequence of SEQ ID NO: 88. In some embodiments, the TCR beta chain construct comprises a complementarity determining region 2 (CDR2), wherein the CDR2 has an amino acid sequence of SEQ ID NO: 89. In some embodiments, the TCR beta chain construct comprises a variable region having at least 80% sequence identity to an amino acid sequence set forth in SEQ ID NO: 115. In some embodiments, the TCR beta chain construct comprises a variable region having at least 90% sequence identity to an amino acid sequence set forth in SEQ ID NO: 115. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 115. In some embodiments, the TCR beta chain construct comprises a variable region having an amino acid sequence set forth in SEQ ID NO: 115. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 112. In some embodiments, the TCR alpha chain construct comprises a variable region having at least 80% sequence identity to an amino acid sequence set forth in SEQ ID NO: 114. In some embodiments, the TCR alpha chain construct comprises a variable region having at least 90% sequence identity to an amino acid sequence set forth in SEQ ID NO: 114. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 114. In some embodiments, the TCR alpha chain construct comprises a variable region having an amino acid sequence set forth in SEQ ID NO: 114.

In some embodiments, the TCR beta chain construct comprises a complementarity determining region 3 (CDR3), wherein the CDR3 has an amino acid sequence of SEQ ID NO: 117. In some embodiments, the TCR beta chain construct comprises a complementarity determining region 1 (CDR1), wherein the CDR1 has an amino acid sequence of SEQ ID NO: 107. In some embodiments, the TCR beta chain construct comprises a complementarity determining region 2 (CDR2), wherein the CDR2 has an amino acid sequence of SEQ ID NO: 108. In some embodiments, the TCR beta chain construct comprises a variable region having at least 80% sequence identity to an amino acid sequence set forth in SEQ ID NO: 119. In some embodiments, the TCR beta chain construct comprises a variable region having at least 90% sequence identity to an amino acid sequence set forth in SEQ ID NO: 119. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 119. In some embodiments, the TCR beta chain construct comprises a variable region having an amino acid sequence set forth in SEQ ID NO: 119. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 116. In some embodiments, the TCR alpha chain construct comprises a variable region having at least 80% sequence identity to an amino acid sequence set forth in SEQ ID NO: 118. In some embodiments, the TCR alpha chain construct comprises a variable region having at least 90% sequence identity to an amino acid sequence set forth in SEQ ID NO: 118. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 118. In some embodiments, the TCR alpha chain construct comprises a variable region having an amino acid sequence set forth in SEQ ID NO: 118.

In some embodiments, the TCR beta chain construct comprises a complementarity determining region 3 (CDR3), wherein the CDR3 has an amino acid sequence of SEQ ID NO: 123. In some embodiments, the TCR beta chain construct comprises a complementarity determining region 1 (CDR1), wherein the CDR1 has an amino acid sequence of SEQ ID NO: 121. In some embodiments, the TCR beta chain construct comprises a complementarity determining region 2 (CDR2), wherein the CDR2 has an amino acid sequence of SEQ ID NO: 122. In some embodiments, the TCR beta chain construct comprises a variable region having at least 80% sequence identity to an amino acid sequence set forth in SEQ ID NO: 125. In some embodiments, the TCR beta chain construct comprises a variable region having at least 90% sequence identity to an amino acid sequence set forth in SEQ ID NO: 125. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 125. In some embodiments, the TCR beta chain construct comprises a variable region having an amino acid sequence set forth in SEQ ID NO: 125. In some embodiments, the TCR further comprises a TCR alpha chain construct having a CDR1, a CDR2, and a CDR3, wherein, the CDR1 has an amino acid sequence set forth in SEQ ID NO: 104; the CDR2 has an amino acid sequence set forth in SEQ ID NO: 105; and, the CDR3 has an amino acid sequence set forth in SEQ ID NO: 120. In some embodiments, the TCR alpha chain construct comprises a variable region having at least 80% sequence identity to an amino acid sequence set forth in SEQ ID NO: 124. In some embodiments, the TCR alpha chain construct comprises a variable region having at least 90% sequence identity to an amino acid sequence set forth in SEQ ID NO: 124. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 124. In some embodiments, the TCR alpha chain construct comprises a variable region having an amino acid sequence set forth in SEQ ID NO: 124. In some embodiments, the epitope from human RAS comprising the mutation G12V is at least 60%, 65%, 70%, 75%, 80%, 85%, 90%, 95%, 96%, 98%, 99% or 100% sequence identical to SEQ ID NO: 133 or 134.

Provided herein is a recombinant nucleic acid encoding a T cell receptor (TCR) comprising a TCR beta chain construct comprising a complementarity determining region 3 (CDR3) having an amino acid sequence set forth in SEQ ID NO: 129. In some embodiments, the TCR beta chain construct comprises a complementarity determining region 1 (CDR1), wherein the CDR1 has an amino acid sequence of SEQ ID NO: 127. In some embodiments, the TCR beta chain construct comprises a complementarity determining region 2 (CDR2), wherein the CDR2 has an amino acid sequence of SEQ ID NO: 128. In some embodiments, the TCR beta chain construct comprises a variable region having at least 80% sequence identity to an amino acid sequence set forth in SEQ ID NO: 131. In some embodiments, the TCR beta chain construct comprises a variable region having at least 90% sequence identity to an amino acid sequence set forth in SEQ ID NO: 131. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 131. In some embodiments, the TCR beta chain construct comprises a variable region having an amino acid sequence set forth in SEQ ID NO: 131. In some embodiments, the TCR further comprises a TCR alpha chain construct having a CDR1, a CDR2, and a CDR3, wherein, the CDR1 has an amino acid sequence set forth in SEQ ID NO: 29; the CDR2 has an amino acid sequence set forth in SEQ ID NO: 30; and, the CDR3 has an amino acid sequence set forth in SEQ ID NO: 126. In some embodiments, the TCR alpha chain construct comprises a variable region having at least 80% sequence identity to an amino acid sequence set forth in SEQ ID NO: 130. In some embodiments, the TCR alpha chain construct comprises a variable region having at least 90% sequence identity to an amino acid sequence set forth in SEQ ID NO: 130. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 130. In some embodiments, the TCR alpha chain construct comprises a variable region having an amino acid sequence set forth in SEQ ID NO: 130. In some embodiments, the TCR binds to an epitope from human RAS comprising a mutation G12D. In some embodiments, the epitope from human RAS comprising the mutation G12D is at least 60%, 65%, 70%, 75%, 80%, 85%, 90%, 95%, 96%, 98%, 99% or 100% sequence identical to SEQ ID NO: 135. In some embodiments, the TCR binds to a complex comprising (i) the epitope from human RAS comprising the mutation G12D and (ii) an MHC protein encoded by an HLA-A11:01 allele.

Provided herein is a recombinant nucleic acid encoding a T cell receptor (TCR) comprising a TCR beta chain construct and a TCR alpha chain construct, wherein the TCR can specifically bind to a mutated epitope from human RAS in complex with a human MHC encoded by an HLA-C05:01 allele with a K_{D} of at most 0.001 nM, at most 0.01 nM, at most 0.1 nM, at most 1 nM, at most 10 nM, at most 100 nM, at most 1000 nM, at most 10000 nM, or at most 100000 nM. The mutated epitope from human RAS can be characterized by a G12D mutation. Provided herein is a recombinant nucleic acid encoding a T cell receptor (TCR) comprising a TCR beta chain construct and a TCR alpha chain construct, wherein the TCR specifically can bind to a mutated epitope from human RAS in complex with a human MHC encoded by an HLA-C05:01 allele with a K_{D} of 0.001 nM, 0.01 nM, 0.1 nM, 1 nM, 10 nM, 100 nM, 1000 nM, 10000 nM, 100000 nM or more. The mutated epitope from human RAS can be characterized by a G12D mutation. In some embodiments, the TCR beta chain construct comprises a complementarity determining region 3 (CDR3), wherein the CDR3 has an amino acid sequence of SEQ ID NO: 90. In some embodiments, the TCR beta chain construct comprises a complementarity determining region 1 (CDR1), wherein the CDR1 has an amino acid sequence of SEQ ID NO: 88. In some embodiments, the TCR beta chain construct comprises a complementarity determining region 2 (CDR2), wherein the CDR2 has an amino acid sequence of SEQ ID NO: 89. In some embodiments, the TCR beta chain construct comprises a variable region having at least 80% sequence identity to an amino acid sequence set forth in SEQ ID NO: 92. In some embodiments, the TCR beta chain construct comprises a variable region having at least 90% sequence identity to an amino acid sequence set forth in SEQ ID NO: 92. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 92. In some embodiments, the TCR beta chain construct comprises a variable region having an amino acid sequence set forth in SEQ ID NO: 92. In some embodiments, the TCR further comprises a TCR alpha chain construct having a CDR1, a CDR2, and a CDR3, wherein, the CDR1 has an amino acid sequence set forth in SEQ ID NO: 85; the CDR2 has an amino acid sequence set forth in SEQ ID NO: 86; and, the CDR3 has an amino acid sequence set forth in SEQ ID NO: 87. In some embodiments, the TCR alpha chain construct comprises a variable region having at least 80% sequence identity to an amino acid sequence set forth in SEQ ID NO: 91. In some embodiments, the TCR alpha chain construct comprises a variable region having at least 90% sequence identity to an amino acid sequence set forth in SEQ ID NO: 91. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 91. In some embodiments, the TCR alpha chain construct comprises a variable region having an amino acid sequence set forth in SEQ ID NO: 91.

In some embodiments, the TCR beta chain construct comprises a complementarity determining region 3 (CDR3), wherein the CDR3 has an amino acid sequence of SEQ ID NO: 95. In some embodiments, the TCR beta chain construct comprises a complementarity determining region 1 (CDR1), wherein the CDR1 has an amino acid sequence of SEQ ID NO: 94. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 97. In some embodiments, the TCR beta chain construct comprises a variable region having at least 90% sequence identity to an amino acid sequence set forth in SEQ ID NO: 97. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 97. In some embodiments, the TCR beta chain construct comprises a variable region having an amino acid sequence set forth in SEQ ID NO: 97. In some embodiments, the TCR further comprises a TCR alpha chain construct having a CDR1, a CDR2, and a CDR3, wherein, the CDR1 has an amino acid sequence set forth in SEQ ID NO: 15; the CDR2 has an amino acid sequence set forth in SEQ ID NO: 16; and, the CDR3 has an amino acid sequence set forth in SEQ ID NO: 93. In some embodiments, the TCR alpha chain construct comprises a variable region having at least 80% sequence identity to an amino acid sequence set forth in SEQ ID NO: 96. In some embodiments, the TCR alpha chain construct comprises a variable region having at least 90% sequence identity to an amino acid sequence set forth in SEQ ID NO: 96. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 96. In some embodiments, the TCR alpha chain construct comprises a variable region having an amino acid sequence set forth in SEQ ID NO: 96.

In some embodiments, the TCR beta chain construct comprises a complementarity determining region 3 (CDR3), wherein the CDR3 has an amino acid sequence of SEQ ID NO: 101. In some embodiments, the TCR beta chain construct comprises a complementarity determining region 1 (CDR1), wherein the CDR1 has an amino acid sequence of SEQ ID NO: 94. In some embodiments, the TCR beta chain construct comprises a complementarity determining region 2 (CDR2), wherein the CDR2 has an amino acid sequence of SEQ ID NO: 81. In some embodiments, the TCR beta chain construct comprises a variable region having at least 80% sequence identity to an amino acid sequence set forth in SEQ ID NO: 103. In some embodiments, the TCR beta chain construct comprises a variable region having at least 90% sequence identity to an amino acid sequence set forth in SEQ ID NO: 103. In some embodiments, the TCR beta chain construct comprises a variable region having at least 60%, 65%, 70%, 75%, 80%, 85%, 90%, 95%, 96%, 98%, or 99% sequence identity to an amino acid sequence set forth in SEQ ID NO: 103. In some embodiments, the TCR beta chain construct comprises a variable region having an amino acid sequence set forth in SEQ ID NO: 103. In some embodiments, the TCR further comprises a TCR alpha chain construct having a CDR1, a CDR2, and a CDR3, wherein, the CDR1 has an amino acid sequence set forth in SEQ ID NO: 98; the CDR2 has an amino acid sequence set forth in SEQ ID NO: 99; and, the CDR3 has an amino acid sequence set forth in SEQ ID NO: 100. In some embodiments, the TCR alpha chain construct comprises a variable region having at least 80% sequence identity to an amino acid sequence set forth in SEQ ID NO: 102. In some embodiments, the TCR alpha chain construct comprises a variable region having at least 90% sequence identity to an amino acid sequence set forth in SEQ ID NO: 102. In some embodiments, the TCR alpha chain construct comprises a variable region having at least 60%, 65%, 70%, 75%, 80%, 85%, 90%, 95%, 96%, 98%, or 99% sequence identity to an amino acid sequence set forth in SEQ ID NO: 102. In some embodiments, the TCR alpha chain construct comprises a variable region having an amino acid sequence set forth in SEQ ID NO: 102. In some embodiments, the epitope from human RAS comprising the mutation G12D is at least 60%, 65%, 70%, 75%, 80%, 85%, 90%, 95%, 96%, 98%, 99% or 100% sequence identical to SEQ ID NO: 132.

In some embodiments, the TCR specifically binds to the mutated epitope with a EC₅₀ of at least 1000 nM.

In various embodiments, the nucleic acid sequence encoding a TCR is codon optimized.

Mutations in any one of the three ras genes, H-ras, K-ras and N-ras are one of the most common events in human tumorigenesis. About 30% of all human tumors are found to carry at least one mutation in any of the canonical ras genes. Ras mutations are evident in, for example, adenocarcinoma of the biliary tract, transitional cell carcinoma of the bladder, breast carcinoma, cervical adenocarcinoma, colon adenocarcinoma, colon adenoma, neuroblastoma (autonomic ganglia), acute myeloid leukemia, chronic myeloid leukemia, chronic myelomonocytic leukemia, juvenile myelomonocytic leukemia, acute lymphoblastic leukemia, Burkitt's lymphoma, Hodgkin's lymphoma, plasma cell myeloma, hepatocellular carcinoma, large cell carcinoma, non-small cell carcinoma, ductal carcinoma, endocrine tumor, prostrate adenocarcinoma, basal cell carcinoma, squamous cell carcinoma, malignant melanoma, angiosarcoma, leiomyosarcoma, liposarcoma, rhabdomyosarcoma, myxoma, malignant fibrous histiocytoma, pleomorphic sarcoma, germinoma, seminoma, anaplastic carcinoma, follicular carcinoma, papillary carcinoma and Hurthle cell carcinoma. Ras mutations are found in cancers affecting many tissues and organs of the body, for example, lung, liver, breast, bladder, colon, cervix, pancreas, prostate gland, stomach, thyroid, testis, soft tissue, skin, and blood.

In various embodiments, the TCR binds to an MHC:RAS peptide complex, wherein the RAS peptide comprises a G12V mutation. In various embodiments, the TCR binds to an MHC:RAS peptide complex, wherein the RAS peptide comprises a G12C mutation. In various embodiments, the TCR binds to an MHC:RAS peptide complex, wherein the RAS peptide comprises a G12D mutation. In various embodiments, the TCR binds to an MHC:RAS peptide complex, wherein the RAS peptide comprises a mutation at Q61. In various embodiments, the TCR binds to an MHC:RAS peptide complex, wherein the RAS peptide comprises a sequence of VVGAVGVGK (SEQ ID NO: 43), VVVGAVGVGK (SEQ ID NO: 44), or AVGVGKSAL (SEQ ID NO: 45). In various embodiments, the TCR binds to an MHC:RAS peptide complex, wherein the RAS peptide comprises a sequence of VVGAVGVGK (SEQ ID NO: 43), or VVVGAVGVGK (SEQ ID NO: 44). In various embodiments, the TCR binds to an MHC:RAS peptide complex, wherein the RAS peptide comprises a sequence of AVGVGKSAL (SEQ ID NO: 45).

In various embodiments, the TCR binds to an MHC:RAS peptide complex, wherein the human MHC is encoded by an HLA-A02:01 allele. In various embodiments, the TCR binds to an MHC:RAS peptide complex, wherein the human MHC is encoded by an HLA-A03:01 allele. In various embodiments, the TCR binds to an MHC:RAS peptide complex, wherein the human MHC is encoded by an HLA-A11:01 allele. In various embodiments, the TCR binds to an MHC:RAS peptide complex, wherein the human MHC is encoded by an HLA-C01:02 allele.

In various embodiments, the TCR binds to an MHC:RAS peptide complex, wherein the human MHC is encoded by an HLA-A11:01 allele and wherein the RAS peptide comprises a sequence of VVGAVGVGK (SEQ ID NO: 43) or VVVGAVGVGK (SEQ ID NO: 44). In various embodiments, the TCR binds to an MHC:RAS peptide complex, wherein the human MHC is encoded by an HLA-C01:02 allele and wherein the RAS peptide comprises a sequence of AVGVGKSAL (SEQ ID NO: 45).

In some embodiments, one TCR as disclosed herein, exhibits a specific binding affinity to an epitope peptide containing a point mutation found in cancer when the epitope peptide is in complex with an MHC encoded by the specific allele; and additionally the TCR may exhibit a different binding affinity to another epitope peptide containing a different point mutation of the same cancer protein, when the epitope peptide is in complex with an MHC encoded by the specific allele, but do not exhibit binding affinity to the WT peptide which does not contain any mutation.

### VI. Delivery of Nucleic Acid or Vector

Nucleic acids encoding TCRs or vectors containing such nucleic acids can be delivered to host cells for expression and processing.

Terms such as “transferring”, “introducing” or “transfecting” are used interchangeably herein and relate to the introduction of nucleic acids, in particular exogenous or heterologous nucleic acids, into a cell.

Cells can be transfected with any carriers with which nucleic acid can be associated, e.g., by forming complexes with the nucleic acid or forming vesicles in which the nucleic acid is enclosed or encapsulated, resulting in increased stability of the nucleic acid compared to naked nucleic acid. Carriers useful according to the present disclosure include, for example, lipid-containing carriers such as cationic lipids, liposomes, in particular cationic liposomes, and micelles, and nanoparticles. Cationic lipids may form complexes with negatively charged nucleic acids. Any cationic lipid may be used according to the present disclosure.

In various embodiments, a nucleic acid encoding a TCR disclosed herein is operably linked to a promoter. Furthermore, the present disclosure provides a vector, e.g., a plasmid, shuttle vector, phagemide, cosmid, expression vector, retroviral vector, adenoviral vector, or particle and/or vector to be used in gene therapy, which comprises one or more of the nucleic acids as disclosed above. A “vector” is a nucleic acid molecule that is capable of transporting another nucleic acid. The vector comprises the nucleic acid insert, which encodes the polylpeptide or protein desired for expression in a cell such as a host cell. For the purposes of the disclosure, an insert may be a nucleic acid encoding a TCR; an alpha chain or a beta chain or both of a TCR. The term “incorporating” a nucleic acid sequence in a vector may mean preparing a suitable expression vector with an insert comprising said nucleic acid sequence. An “expression vector” is a vector that can direct the expression of a protein encoded by one or more genes carried by the vector when it is present in the appropriate environment. “Retroviruses” are viruses having an RNA genome. “Gammaretrovirus” refers to a genus of the retroviridae family. Exemplary gammaretroviruses include, but are not limited to, mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Several examples of lentivinuses include HIV (human immunodeficiency virus: including. HIV type 1, and HIV type 2); equine infectious anemia virus; feline immunodeficiency virus (FIV); bovine immune deficiency virus (BIV); and simian immunodeficiency virus (SIV). A vector that encodes a core virus is also known as a “viral vector.” There are a large number of available viral vectors that are suitable for use with the invention, including those identified for human gene therapy applications, such as those described by Pfeifer and Verma (Pfeifer, A, and I. M. Verma. 2001. Ann. Rev. Genomics Hum. Genet. 2:177-211). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and maedi/visna virus. Methods of using retroviral and lentiviral viral vectors and packaging cells for transducing mammalian target cells with viral particles containing TCRs transgenes are well known in the art and have been previous described, for example, in U.S. Pat. No. 8,119,772; Walchli et al., 2011, PLoS One 6327930; Zhao et al., J. Immunol., 2005, 174:44-15-4423; Engels et al, 2003, Hum. Gene Ther. 14:1155-68; Frecha et al., 2010, Mol. Ther. 18:1748-57; Verhoeyen et al., 2009, Methods Mol. Biol. 506:97-114. Retroviral and lentiviral vector constructs and expression systems are also commercially available. In some embodiments, a viral vector is used to introduce the non-endogenous nucleic acid sequence encoding TCRα chain specific for the peptide antigen into the hematopoietic progenitor cells. The viral vector may be a retroviral vector or a lentiviral vector. The viral vector may also include a nucleic acid sequence encoding a marker for transduction. Transduction markers for viral vectors are known in the art and include selection markers, which may confer drug resistance, or detectable markers, such as fluorescent markers or cell surface proteins that can be detected by methods such as flow cytometry. Where the viral vector genome comprises more than one nucleic acid sequence to be expressed in the host cell as separate transcripts, the viral vector may also comprise additional sequence between the two (or more) transcripts allowing bicistronic or multicistronic expression. Examples of such sequences used in viral vectors include internal ribosome entry sites (IRES), furin cleavage sites, viral 2A peptide. Other vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., 1998, Gene Ther. 5: 1517-30). Other vectors include those derived from baculoviruses and alpha-viruses. (Jolly D J. 1999. Emerging viral vectors. pp 209-40 in Friedmann T. ed. 1999. The development of human gene therapy. New York: Cold Spring Harbor Lab).

A vector may include nucleic acid sequences that permit the nucleic acid to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

In some embodiments, provided herein is a vector comprising a nucleic acid encoding a TCR disclosed herein. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccina virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

Presented herein are constructs, for example, nucleic acid constructs that encode an alpha chain and a beta chain of a TCR for expression in a cell. In some embodiments, the constructs comprise one or more polynucleotides encoding a TCR alpha chain and a TCR beta chain. In some embodiments, the polynucleotides are incorporated in a suitable vector. In some embodiments, the polynucleotides encoding the alpha chain and the beta chain are incorporated in the same vector. In some embodiments, the polynucleotides encoding the alpha chain and the beta chain are incorporated in different vectors, and both vectors are delivered for expression in the single cell.

In some embodiments, a cell may be transduced or transfected with a nucleic acid encoding a TCR, wherein the cell is capable of expressing the TCR and the cell is used as a therapeutic. In some embodiments the cell is derived from a subject or a host, wherein the subject or the host is a human. In some embodiments, the subject or the host comprises a cell having a mutation in an epitope, and the TCR expressed in the cell is capable of binding specifically to the epitope having the mutation. In some embodiments, the cell is a lymphocyte cell. In some embodiments, the T-lymphocyte. In some embodiments, the cell is a lymphocytic precursor cell. In some embodiments, the cell is a T lymphocyte precursor cell. In some embodiments, the cell is a T lymphocyte progenitor cell. In some embodiments the cell is a thymocyte.

In some embodiments, the T cells are immature T cells. In some embodiments, the T cells are antigen naive T cells. The host cell may be cultured ex vivo for 1, 2, 3, 4, 5 or more days for monitoring and recover after transfection or transduction with the polynucleotide(s) encoding the TCR.

### VII. Neoantigens

The TCRs disclosed herein are specific to immunogenic neoantigens. In some embodiments, the neoantigen peptide is from RAS. In some embodiments, the neoantigen peptide is from GATA3. In some embodiments, the neoantigen peptide is from BTK. In some embodiments, the neoantigen peptide is from TMPRSS2:ERG. In some embodiments, one or more neoantigen peptides are loaded on to APCs, wherein the peptide loaded APCs are then used to stimulate T cells to produce antigen specific T cells. In some embodiments, the APCs used for peptide loading are dendritic cells. Immunogenic neoantigen sequences can be identified by any suitable method known in the art.

In various embodiments, the neoantigen comprises an epitope. In both animals and humans, mutated epitopes can be potentially effective in inducing an immune response or activating T cells. In some embodiments, the epitope comprises a mutation. In some embodiments, the mutation is selected from the group consisting of a point mutation, a splice-site mutation, a frameshift mutation, a read-through mutation, a gene fusion mutation, and any combination thereof.

In some embodiments, the epitope has a length of at least 8 amino acids. In some embodiments, the epitope has a length of at least 16 amino acids. In some embodiments, the epitope has a length of from 8-25 amino acids. In some embodiments, the epitope has a length of from 8-12 amino acids. In some embodiments, the epitope has a length of from 16-25 amino acids. In some embodiments, the epitope has a length of 7, 8, 9, 10, 11, 12, 13, 14, 15, 16, 17, 18, 19, 20, 21, 22, 23, 24, or 25 amino acids.

In certain embodiments, a neoantigen or epitope thereof can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein. In specific embodiments, a neoantigen or epitope thereof is equal to or less than 100 amino acids.

In some embodiments, a neoantigen or epitope thereof for MHC Class I is 13 residues or less in length and usually consists of between about 8 and about 11 residues, particularly 9 or 10 residues. In some embodiments, a neoantigen or epitope thereof for MHC Class II is 9-24 residues in length.

In some embodiments, neoantigens bind an HLA protein (e.g., HLA class I or HLA class II). In specific embodiments neoantigens bind an HLA protein with greater affinity than a corresponding wild-type peptide. In specific embodiments, the neoantigenic peptide or polypeptide has an IC₅₀ of at least less than 5000 nM, at least less than 500 nM, at least less than 100 nM, at least less than 50 nM or less.

In some embodiments, the epitope binds to the human MHC with a greater affinity than a corresponding wild-type epitope. In some embodiments, the epitope binds to the human MHC with a K_{D} or an IC₅₀ less than 500 nM, 250 nM, 150 nM, 100 nM, 50 nM, 25 nM, 10 nM, or 5 nM. In some embodiments, the epitope binds to the human MHC with a K_{D} or an IC₅₀ of at most 500 nM, 250 nM, 150 nM, 100 nM, 50 nM, 25 nM, 10 nM, or 5 nM. In some embodiments, the epitope binds to the human MHC with a EC₅₀ or avidity less than 100000 nM, 10000 nM, 1000 nM, 500 nM, 250 nM, 150 nM, 100 nM, 50 nM, 25 nM, 10 nM, or 5 nM. In some embodiments, the epitope binds to the human MHC with a EC₅₀ or avidity of least about 100000 nM, 10000 nM, 1000 nM, 500 nM, 250 nM, 150 nM, 100 nM, 50 nM, 25 nM, 10 nM, or 5 nM. In some embodiments, the epitope comprises a mutation, wherein the mutation is not present in non-cancer cells of a subject. In some embodiments, the epitope is encoded by a gene or an expressed gene of a subject's cancer cells. In some embodiments, the TCR binds to an HLA-peptide complex with a K_{D} or an IC₅₀ of less than 500 nM, 250 nM, 150 nM, 100 nM, 50 nM, 25 nM, 10 nM, or 5 nM. In some embodiments, the TCR binds to an HLA-peptide complex with a K_{D} or an IC₅₀ of at most 500 nM, 250 nM, 150 nM, 100 nM, 50 nM, 25 nM, 10 nM, or 5 nM. In some embodiments, the TCR binds to an HLA-peptide complex with a EC₅₀ or avidity less than 100000 nM, 10000 nM, 1000 nM, 500 nM, 250 nM, 150 nM, 100 nM, 50 nM, 25 nM, 10 nM, or 5 nM. In some embodiments, the TCR binds to an HLA-peptide complex with a EC₅₀ or avidity of least about 100000 nM, 10000 nM, 1000 nM, 500 nM, 250 nM, 150 nM, 100 nM, 50 nM, 25 nM, 10 nM, or 5 nM.

In some embodiments, the neoantigenic peptide can be from about 8 and about 50 amino acid residues in length, or from about 8 and about 30, from about 8 and about 20, from about 8 and about 18, from about 8 and about 15, or from about 8 and about 12 amino acid residues in length. In some embodiments, the neoantigenic peptide can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In some embodiments, the neoantigenic peptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the neoantigenic peptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500 or more amino acid residues in length. In some embodiments, the neoantigenic peptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the neoantigenic peptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, the neoantigenic peptide has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids.

In some embodiments, the neoantigenic peptide has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids.

In some embodiments, the neoantigenic peptide can have a pI value of about 0.5 and about 12, about 2 and about 10, or about 4 and about 8. In some embodiments, the neoantigenic peptide can have a pI value of at least 4.5, 5, 5.5, 6, 6.5, 7, 7.5, or more. In some embodiments, the neoantigenic peptide can have a pI value of at most 4.5, 5, 5.5, 6, 6.5, 7, 7.5, or less.

In some embodiments, the neoantigenic peptide can have an HLA binding affinity of between about 1 pM and about 1 mM, about 100 pM and about 500 μM, about 500 pM and about 10 μM, about 1 nM and about 1 μM, or about 10 nM and about 1 μM. In some embodiments, the neoantigenic peptide can have an HLA binding affinity of at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900 μM, or more. In some embodiments, the neoantigenic peptide can have an HLA binding affinity of at most 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900 μM.

In some embodiments, a neoantigenic peptide described herein can comprise carriers such as those well known in the art, e.g., thyroglobulin, albumins such as human serum albumin, tetanus toxoid, polyamino acid residues such as poly L-lysine, poly L-glutamic acid, influenza virus proteins, hepatitis B virus core protein, and the like.

In some embodiments, a neoantigenic peptide described herein can be modified by terminal-NH2 acylation, e.g., by alkanoyl (C1-C20) or thioglycolyl acetylation, terminal-carboxyl amidation, e.g., ammonia, methylamine, etc. In some embodiments, these modifications can provide sites for linking to a support or other molecule.

In some embodiments, a neoantigenic peptide described herein can contain modifications such as but not limited to glycosylation, side chain oxidation, biotinylation, phosphorylation, addition of a surface active material, e.g., a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, a neoantigenic peptide described herein can contain substitutions to modify a physical property (e.g., stability or solubility) of the resulting peptide. For example, neoantigenic peptides can be modified by the substitution of a cysteine (C) with α-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and cross-binding capability in certain instances. Substitution of cysteine with α-amino butyric acid can occur at any residue of a neoantigenic peptide, e.g., at either anchor or non-anchor positions of an epitope or analog within a peptide, or at other positions of a peptide.

In some embodiments, a neoantigenic peptide described herein can comprise amino acid mimetics or unnatural amino acid residues, e.g. D- or L-naphylalanine; D- or L-phenylglycine; D- or L-2-thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-.rho.-fluorophenylalanine; D- or L-.rho.-biphenyl-phenylalanine; D- or L-.rho.-methoxybiphenylphenylalanine; D- or L-2-indole(allyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides that have various amino acid mimetics or unnatural amino acid residues are particularly useful, as they tend to manifest increased stability in vivo. Such peptides can also possess improved shelf-life or manufacturing properties.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides described herein is conveniently determined using a 25% human serum (v/v) assay. The protocol is as follows: pooled human serum (Type AB, non-heat inactivated) is dilapidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

In some embodiments, a neoantigenic peptide described herein can be prepared synthetically, by recombinant DNA technology or chemical synthesis, or can be isolated from natural sources such as native tumors or pathogenic organisms. Epitopes can be synthesized individually or joined directly or indirectly in a peptide. Although a neoantigenic peptide described herein will be substantially free of other naturally occurring host cell proteins and fragments thereof, in some embodiments, the peptide can be synthetically conjugated to be joined to native fragments or particles.

In some embodiments, a neoantigenic peptide described herein can be prepared in a wide variety of ways. In some embodiments, the peptides can be synthesized in solution or on a solid support according to conventional techniques. Various automatic synthesizers are commercially available and can be used according to known protocols. (See, for example, Stewart & Young, SOLID PHASE PEPTIDE SYNTHESIS, 2D. ED., Pierce Chemical Co., 1984). Further, individual peptides can be joined using chemical ligation to produce larger peptides that are still within the bounds of the invention.

Alternatively, recombinant DNA technology can be employed wherein a nucleotide sequence which encodes a peptide inserted into an expression vector, transformed, or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Thus, recombinant peptides, which comprise or consist of one or more epitopes described herein, can be used to present the appropriate T cell epitope.

### VII. Pharmaceutical Compositions

Pharmaceutical compositions can be formulated using one or more physiologically acceptable carriers including excipients and auxiliaries which facilitate processing of the active agents into preparations which can be used pharmaceutically. Proper formulation can be dependent upon the route of administration chosen. Any of the well-known techniques, carriers, and excipients can be used as suitable and as understood in the art.

In some cases, a pharmaceutical composition is formulated as cell based therapeutic, e.g., a T cell therapeutics. In some embodiments, a pharmaceutical composition comprises a peptide-based therapy, a nucleic acid-based therapy, an antibody-based therapy, and/or a cell-based therapy. In some embodiments, a pharmaceutical composition comprises a peptide-based therapeutic, or nucleic acid based therapeutic in which the nucleic acid encodes the polypeptides. A composition can comprise T cells specific for two or more immunogenic antigen or neoantigen peptides. In some embodiments, the T cell specific therapeutic may be supplemented by one or more additional therapies.

In some embodiments, a pharmaceutical composition comprising: a nucleic acid encoding a TCR targeting a neoantigen disclosed herein, a vector containing the nucleic acid, the protein encoded by the nucleic acid, or a host cell comprising the nucleic acid, the protein, or the vector; and a pharmaceutically acceptable excipient or diluent. In some embodiments, the pharmaceutical composition further comprises an immunomodulatory agent or an adjuvant. In some embodiments, the immunomodulatory agent is a cytokine. In some embodiments, the adjuvant is poly I:C.

Also provided herein is the use of the pharmaceutical compositions in treating an immune disease or cancer.

Pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration. Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington's Pharmaceutical Sciences (18th ed. A. Gennaro, Mack Publishing Co., Easton, PA 1990). One example of carrier is physiological saline. A pharmaceutically acceptable carrier is a pharmaceutically acceptable material, composition, or vehicle, such as a liquid or solid filler, diluent, excipient, solvent, or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the other ingredients.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (e.g., systemic or local). Thus, compositions include carriers, diluents, or excipients suitable for administration by various routes.

In some embodiments, a pharmaceutical composition can further comprise an acceptable additive in order to improve the stability of the composition. Acceptable additives may not alter the specific activity of the active agent, e.g., immune cells. Examples of acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

In some embodiments the pharmaceutical composition comprises a therapeutic which is a T cell expressing one or more polynucleotides, encoding a T cell receptor. In some embodiments, the pharmaceutical composition comprises physiologically acceptable carrier suitable for a cell suspension.

The pharmaceutical composition can be administered, for example, by injection. Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid, and thimerosal. Isotonic agents, for example, sugars, polyalcohols such as mannitol, sorbitol, and sodium chloride can be included in the composition. The resulting solutions can be packaged for use as is or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Pharmaceutical compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the pharmaceutical compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The pharmaceutical compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

In some embodiments, the present disclosure is directed to an immunogenic composition, e.g., a pharmaceutical composition capable of raising a neoantigen-specific response (e.g., a humoral or cell-mediated immune response). In some embodiments, the immunogenic composition comprises neoantigen therapeutics (e.g., peptides, polynucleotides, TCR, CAR, cells containing TCR or CAR, dendritic cell containing polypeptide, dendritic cell containing polynucleotide, antibody, etc.) described herein corresponding to a tumor specific antigen or neoantigen.

In some embodiments, a pharmaceutical composition described herein is capable of raising a specific cytotoxic T cells response, specific helper T cell response, or a B cell response.

In some embodiments, antigen polypeptides or polynucleotides can be provided in antigen presenting cells (e.g., dendritic cells) containing such polypeptides or polynucleotides. In other embodiments, such antigen presenting cells are used to stimulate T cells for use in patients.

In some embodiments, the antigen presenting cells are dendritic cells. In related embodiments, the dendritic cells are autologous dendritic cells that are pulsed with the neoantigen peptide or nucleic acid. The neoantigen peptide can be any suitable peptide that gives rise to an appropriate T cell response. In some embodiments, the T cell is a CTL. In some embodiments, the T cell is an HTL. Thus, one embodiment of the present disclosure is an immunogenic composition containing at least one antigen presenting cell (e.g., a dendritic cell) that is pulsed or loaded with one or more neoantigen polypeptides or polynucleotides described herein. In some embodiments, such APCs are autologous (e.g., autologous dendritic cells). Alternatively, peripheral blood mononuclear cells (PBMCs) isolated from a patient can be loaded with neoantigen peptides or polynucleotides ex vivo. In related embodiments, such APCs or PBMCs are injected back into the patient. The polynucleotide can be any suitable polynucleotide that is capable of transducing the dendritic cell, thus resulting in the presentation of a neoantigen peptide and induction of immunity. In some embodiments, such antigen presenting cells (APCs) (e.g., dendritic cells) or peripheral blood mononuclear cells (PBMCs) are used to stimulate a T cell (e.g., an autologous T cell, or an allogeneic T cell). In related embodiments, the T cell is a CTL. In other related embodiments, the T cell is an HTL. In some embodiments, the T cells are CD8⁺ T cells. In some embodiments, the T cells are CD4⁺ T cells. Such T cells are then injected into the patient. In some embodiments, CTL is injected into the patient. In some embodiments, HTL is injected into the patient. In some embodiments, both CTL and HTL are injected into the patient. Administration of either therapeutic can be performed simultaneously or sequentially and in any order.

In some embodiments, pharmaceutical compositions (e.g., immunogenic compositions) described herein for therapeutic treatment can be formulated for parenteral, topical, nasal, oral or local administration. In some embodiments, the pharmaceutical compositions described herein are administered parenterally, e.g., intravenously, subcutaneously, intradermally, or intramuscularly. In some embodiments, the composition can be administered intratumorally. The compositions can be administered at the site of surgical excision to induce a local immune response to the tumor. In some embodiments, described herein are compositions for parenteral administration which comprise a solution of the neoantigen peptides and immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid, and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The ability of an adjuvant to increase the immune response to an antigen is typically manifested by a significant increase in immune-mediated reaction, or reduction in disease symptoms. For example, an increase in humoral immunity can be manifested by a significant increase in the titer of antibodies raised to the antigen, and an increase in T cell activity can be manifested in increased cell proliferation, or cellular cytotoxicity, or cytokine secretion. An adjuvant can also alter an immune response, for example, by changing a primarily humoral or T helper 2 response into a primarily cellular, or T helper 1 response.

Suitable adjuvants are known in the art (see, WO 2015/095811) and include, but are not limited to poly(I:C), poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel® vector system, PLG microparticles, resiquimod, SRL172, virosomes and other virus-like particles, YF-17D, VEGF trap, R848, β-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described (Dupuis M, et al., Cell Immunol. 1998; 186(1):18-27; Allison A C; Dev Biol Stand. 1998; 92:3-11) (Mosca et al. Frontiers in Bioscience, 2007; 12:4050-4060) (Gamvrellis et al. Immunol & Cell Biol. 2004; 82: 506-516). Also, cytokines can be used. Several cytokines have been directly linked to influencing dendritic cell migration to lymphoid tissues (e.g., TNF-α), accelerating the maturation of dendritic cells into efficient antigen-presenting cells for T-lymphocytes (e.g., GM-CSF, PGE1, PGE2, IL-1, IL-1β, IL-4, IL-6 and CD40L) (U.S. Pat. No. 5,849,589 incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a therapeutic setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell immunogenic pharmaceutical compositions, autologous cellular immunogenic pharmaceutical compositions, and polysaccharide conjugates in both prophylactic and therapeutic immunogenic pharmaceutical compositions. Importantly, it enhances dendritic cell maturation and differentiation, resulting in enhanced activation of TH1 cells and strong cytotoxic T-lymphocyte (CTL) generation, even in the absence of CD4+ T cell help. The TH1 bias induced by TLR9 stimulation is maintained even in the presence of adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially useful for inducing a strong response when the antigen is relatively weak. They can also accelerate the immune response and enabled the antigen doses to be reduced with comparable antibody responses to the full-dose immunogenic pharmaceutical composition without CpG in some experiments (Arthur M. Krieg, Nature Reviews, Drug Discovery, 5, June 2006, 471-484). U.S. Pat. No. 6,406,705 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, DE), which is a component of the pharmaceutical composition described herein. Other TLR binding molecules such as RNA binding TLR7, TLR8 and/or TLR9 can also be used.

Other examples of useful adjuvants include, but are not limited to, chemically modified CpGs (e.g. CpR, Idera), Poly(I:C)(e.g., polyL:CI2U), polyIC:LC, non-CpG bacterial DNA or RNA, ssRNA40 for TLR8, as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which can act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

In some embodiments, an immunogenic composition according to the present disclosure can comprise more than one different adjuvant. Furthermore, the invention encompasses a pharmaceutical composition comprising any adjuvant substance including any of the above or combinations thereof. In some embodiments, the immunogenic composition comprises neoantigen therapeutics (e.g., peptides, polynucleotides, TCR, CAR, cells containing TCR or CAR, dendritic cell containing polypeptide, dendritic cell containing polynucleotide, antibody, etc.) and the adjuvant can be administered separately in any appropriate sequence.

Lipidation can be classified into several different types, such as N-myristoylation, palmitoylation, GPI-anchor addition, prenylation, and several additional types of modifications. N-myristoylation is the covalent attachment of myristate, a C14 saturated acid, to a glycine residue. Palmitoylation is thioester linkage of long-chain fatty acids (C16) to cysteine residues. GPI-anchor addition is glycosyl-phosphatidylinositol (GPI) linkage via amide bond. Prenylation is the thioether linkage of an isoprenoid lipid (e.g., farnesyl (C-15), geranylgeranyl (C-20)) to cysteine residues. Additional types of modifications can include attachment of S-diacylglycerol by a sulfur atom of cysteines, 0-octanoyl conjugation via serine or threonine residues, S-archaeol conjugation to cysteine residues, and cholesterol attachment.

Fatty acids for generating a lipidated peptides can include C2 to C30 saturated, monounsaturated, or polyunsaturated fatty acyl groups. Exemplary fatty acids can include palmitoyl, myristoyl, stearoyl and decanoyl groups. In some instances, a lipid moiety that has adjuvant property is attached to a polypeptide of interest to elicit or enhance immunogenicity in the absence of an extrinsic adjuvant. A lipidated peptide or lipopeptide can be referred to as a self-adjuvant lipopeptide. Any of the fatty acids described above and elsewhere herein can elicit or enhance immunogenicity of a polypeptide of interest. A fatty acid that can elicit or enhance immunogenicity can include palmitoyl, myristoyl, stearoyl, lauroyl, octanoyl, and decanoyl groups.

Polypeptides such as naked peptides or lipidated peptides can be incorporated into a liposome. Sometimes, lipidated peptides can be incorporated into a liposome. For example, the lipid portion of the lipidated peptide can spontaneously integrate into the lipid bilayer of a liposome. Thus, a lipopeptide can be presented on the “surface” of a liposome.

Liposome can also be used to deliver nucleic acids into a cell. The nucleic acid of interest comprises one or more sequences encoding a T cell receptor. Liposomes may be used to deliver a DNA or an RNA. Liposomes may be used to deliver a nucleic acid incorporated in a vector. The nucleic acid may be 50-200,000 nucleotides long, or may be 100-500,000 nucleotides long, or may be 20-500,000 nucleotides long. Exemplary liposomes suitable for incorporation in the formulations include, and are not limited to, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

Depending on the method of preparation, liposomes can be unilamellar or multilamellar, and can vary in size with diameters ranging from about 0.02 μm to greater than about 10 μm. Liposomes can adsorb many types of cells and then release an incorporated agent (e.g., a peptide described herein). In some cases, the liposomes fuse with the target cell, whereby the contents of the liposome then empty into the target cell. A liposome can be endocytosed by cells that are phagocytic. Endocytosis can be followed by intralysosomal degradation of liposomal lipids and release of the encapsulated agents.

The liposomes provided herein can also comprise carrier lipids. In some embodiments, the carrier lipids are phospholipids. Carrier lipids capable of forming liposomes include, but are not limited to dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or combinations thereof. In some embodiments, the liposomes further comprise a sterol (e.g., cholesterol) which modulates liposome formation. The carrier lipids can be any known non-phosphate polar lipids.

A pharmaceutical composition can be encapsulated within liposomes using well-known technology. Biodegradable microspheres can also be employed as carriers for the pharmaceutical compositions of this invention.

The pharmaceutical composition can be administered in liposomes or microspheres (or microparticles). Methods for preparing liposomes and microspheres for administration to a patient are well known to those of skill in the art. Essentially, material is dissolved in an aqueous solution, the appropriate phospholipids and lipids added, along with surfactants if required, and the material dialyzed or sonicated, as necessary.

Microspheres formed of polymers or proteins are well known to those skilled in the art and can be tailored for passage through the gastrointestinal tract directly into the blood stream. Alternatively, the compound can be incorporated and the microspheres, or composite of microspheres, implanted for slow release over a period of time ranging from days to months.

Cell-based immunogenic pharmaceutical compositions can also be administered to a subject. For example, an antigen presenting cell (APC) based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein.

The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

An adjuvant can be used to enhance the immune response (humoral and/or cellular) elicited in a patient receiving the immunogenic pharmaceutical composition. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5, and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Adjuvant can also comprise stimulatory molecules such as cytokines. Non-limiting examples of cytokines include: CCL20, α-interferon (IFNα), 0-interferon (IFNβ), γ-interferon (IFNγ), platelet derived growth factor (PDGF), TNFα, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-I, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-1, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR9 agonists and are not limited to small molecule modulators of toll-like receptors such as Imiquimod. Sometimes, an adjuvant is selected from bacteria toxoids, poly oxypropylene-poly oxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolizable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

In some instances, an immunogenic pharmaceutical composition can include carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In another instances, the pharmaceutical preparation is substantially free of preservatives. In other instances, the pharmaceutical preparation can contain at least one preservative. It will be recognized that, while any suitable carrier known to those of ordinary skill in the art can be employed to administer the pharmaceutical compositions described herein, the type of carrier will vary depending on the mode of administration.

An immunogenic pharmaceutical composition can include preservatives such as thiomersal or 2-phenoxyethanol. In some instances, the immunogenic pharmaceutical composition is substantially free from (e.g., <10 μg/mL) mercurial material e.g., thiomersal-free. α-Tocopherol succinate may be used as an alternative to mercurial compounds.

For controlling the tonicity, a physiological salt such as sodium salt can be included in the immunogenic pharmaceutical composition. Other salts can include potassium chloride, potassium dihydrogen phosphate, disodium phosphate, and/or magnesium chloride, or the like.

An immunogenic pharmaceutical composition can have an osmolality of between 200 mOsm/kg and 400 mOsm/kg, between 240-360 mOsm/kg, or within the range of 290-310 mOsm/kg.

An immunogenic pharmaceutical composition can comprise one or more buffers, such as a Tris buffer; a borate buffer; a succinate buffer; a histidine buffer (particularly with an aluminum hydroxide adjuvant); or a citrate buffer. Buffers, in some cases, are included in the 5-20 or 10-50 mM range.

The pH of the immunogenic pharmaceutical composition can be between about 5.0 and about 8.5, between about 6.0 and about 8.0, between about 6.5 and about 7.5, or between about 7.0 and about 7.8.

An immunogenic pharmaceutical composition can be sterile. The immunogenic pharmaceutical composition can be non-pyrogenic e.g., containing <1 EU (endotoxin unit, a standard measure) per dose, and can be <0.1 EU per dose. The composition can be gluten free.

An immunogenic pharmaceutical composition can include detergent e.g., a polyoxyethylene sorbitan ester surfactant (known as ‘Tweens’), or an octoxynol (such as octoxynol-9 (Triton X-100) or t-octylphenoxypolyethoxyethanol). The detergent can be present only at trace amounts. The immunogenic pharmaceutical composition can include less than 1 mg/mL of each of octoxynol-10 and polysorbate 80. Other residual components in trace amounts can be antibiotics (e.g., neomycin, kanamycin, polymyxin B).

An immunogenic pharmaceutical composition can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration.

Pharmaceutical compositions comprising, for example, an active agent such as immune cells disclosed herein, in combination with one or more adjuvants can be formulated to comprise certain molar ratios. For example, molar ratios of about 99:1 to about 1:99 of an active agent such as an immune cell described herein, in combination with one or more adjuvants can be used. In some instances, the range of molar ratios of an active agent such as an immune cell described herein, in combination with one or more adjuvants can be selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of an active agent such as an immune cell described herein, in combination with one or more adjuvants can be about 1:9, and in some cases can be about 1:1. The active agent such as an immune cell described herein, in combination with one or more adjuvants can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

In some instances, an immunogenic pharmaceutical composition can be administered with an additional agent. The choice of the additional agent can depend, at least in part, on the condition being treated. The additional agent can include, for example, a checkpoint inhibitor agent such as an anti-PD1, anti-CTLA4, anti-PD-L1, anti CD40, or anti-TIM3 agent (e.g., an anti-PD1, anti-CTLA4, anti-PD-L1, anti CD40, or anti-TIM3 antibody); or any agents having a therapeutic effect for a pathogen infection (e.g. viral infection), including, e.g., drugs used to treat inflammatory conditions such as an NSAID, e.g., ibuprofen, naproxen, acetaminophen, ketoprofen, or aspirin. For example, the checkpoint inhibitor can be a PD-1/PD-L1 antagonist selected from the group consisting of nivolumab (ONO-4538/BMS-936558, MDX1 106, OPDIVO), pembrolizumab (MK-3475, KEYTRUDA), pidilizumab (CT-011), and MPDL3280A (ROCHE). As another example, formulations can additionally contain one or more supplements, such as vitamin C, E, or other anti-oxidants.

A pharmaceutical composition comprising an active agent such as an immune cell described herein, in combination with one or more adjuvants can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The agent(s) described herein can be delivered to a patient using a number of routes or modes of administration, including oral, buccal, topical, rectal, transdermal, transmucosal, subcutaneous, intravenous, and intramuscular applications, as well as by inhalation.

The active agents can be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly(lactic-co-glycolic)acid. These materials can be made into micro or nanospheres, loaded with drug and further coated or derivatized to provide superior sustained release performance. Vehicles suitable for periocular or intraocular injection include, for example, suspensions of therapeutic agent in injection grade water, liposomes, and vehicles suitable for lipophilic substances. Other vehicles for periocular or intraocular injection are well known in the art.

In some instances, pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

When administration is by injection, the active agent can be formulated in aqueous solutions, specifically in physiologically compatible buffers such as Hanks solution, Ringer's solution, or physiological saline buffer. The solution can contain formulatory agents such as suspending, stabilizing and/or dispersing agents. In another embodiment, the pharmaceutical composition does not comprise an adjuvant or any other substance added to enhance the immune response.

In addition to the formulations described previously, the active agents can also be formulated as a depot preparation. Such long acting formulations can be administered by implantation or transcutaneous delivery (for example subcutaneously or intramuscularly), intramuscular injection or use of a transdermal patch. Thus, for example, the agents can be formulated with suitable polymeric or hydrophobic materials (for example as an emulsion in an acceptable oil) or ion exchange resins, or as sparingly soluble derivatives, for example, as a sparingly soluble salt.

In some cases, pharmaceutical compositions comprising one or more agents exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In another embodiment, local/topical formulations comprising a transporter, carrier, or ion channel inhibitor are used to treat epidermal or mucosal viral infections.

Pharmaceutical compositions can contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. The amounts of these various adjuvants are those conventionally used in the fields considered and, for example, are from about 0.01% to about 20% of the total weight of the composition. Depending on their nature, these adjuvants can be introduced into the fatty phase, into the aqueous phase and/or into the lipid vesicles.

### IX. Methods of Treatment

Provided herein are method of using any of the nucleic acid disclosed above, the vector containing any of the nucleic acid sequence disclosed above, the protein encoded by any of the nucleic acid disclosed above, or the host cell disclosed above, for manufacture of a medicament for treating an immune disease or cancer.

Also provided herein are methods of treating a subject with a disease, disorder, or condition. A method of treatment can comprise administering a pharmaceutical composition disclosed herein to a subject with a disease, disorder, or condition. The present disclosure provides methods of treatment comprising an immunogenic therapy. Methods of treatment for a disease (such as cancer or a viral infection) are provided. A method can comprise administering to a subject an effective amount of a pharmaceutical composition comprising an immunogenic antigen specific T cells. In some embodiments, the antigen comprises a tumor antigen.

In some embodiments, the method of treating a subject with a disease or condition comprises administering to the subject the pharmaceutical composition disclosed herein. In some embodiments, the method is a method of preventing resistance to a cancer therapy, wherein the method comprises administering to a subject in need thereof the pharmaceutical composition disclosed herein. In some embodiments, the method is a method of inducing an immune response, wherein the method comprises administering to a subject in need thereof the pharmaceutical composition disclosed herein. In some embodiments, the immune response is a humoral response. In some embodiments, the immune response is a cytotoxic T cell response.

In some embodiments, the subject has cancer, wherein the cancer is selected from the group consisting of melanoma, ovarian cancer, lung cancer, prostate cancer, breast cancer, colorectal cancer, endometrial cancer, and chronic lymphocytic leukemia (CLL).

In some embodiments, the subject has a breast cancer that is resistant to anti-estrogen therapy. In some embodiments, the breast cancer expresses an estrogen receptor with a mutation. In some embodiments, the subject has a CLL that is resistant to ibrutinib therapy. In some embodiments, the CLL expresses a Bruton tyrosine kinase with a mutation, such as a C481S mutation. In some embodiments, the subject has a lung cancer that is resistant to a tyrosine kinase inhibitor. In some embodiments, the lung cancer expresses an epidermal growth factor receptor (EGFR) with a mutation, such as a T790M, L792F, or C797S mutation.

In some embodiments, the method further comprises administering at least one additional therapeutic agent or modality. In some embodiments, the at least one additional therapeutic agent or modality is surgery, a checkpoint inhibitor, an antibody, or fragment thereof, a chemotherapeutic agent, radiation, a vaccine, a small molecule, a T cell, a vector, and APC, a polynucleotide, an oncolytic virus, or any combination thereof. In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering the pharmaceutical composition disclosed herein.

In some other aspects, provided here is use of a pharmaceutical composition for the manufacture of a medicament for use in therapy. In some embodiments, a method of treatment comprises administering to a subject an effective amount of T cells specifically recognizing an immunogenic neoantigen peptide. In some embodiments, a method of treatment comprises administering to a subject an effective amount of a TCR that specifically recognizes an immunogenic neoantigen peptide, such as a TCR expressed in a T cell.

In some embodiments, the cancer is selected from the group consisting of carcinoma, lymphoma, blastoma, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), abnormal vascular proliferation associated with phakomatoses, edema, Meigs' syndrome, and combinations thereof.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterus); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the subject has a breast cancer that is resistant to anti-estrogen therapy, is an MSI breast cancer, is a metastatic breast cancer, is a Her2 negative breast cancer, is a Her2 positive breast cancer, is an ER negative breast cancer, is an ER positive breast cancer or any combination thereof.

In some embodiments, the breast cancer expresses an estrogen receptor with a mutation.

In some embodiments, the cancer is recurrent or metastatic breast cancer. In some embodiments, a cancer to be treated by the methods of treatment of the present disclosure is triple negative breast cancer (TNBC).

The pharmaceutical compositions provided herein may be used alone or in combination with conventional therapeutic regimens such as surgery, irradiation, chemotherapy and/or bone marrow transplantation (autologous, syngeneic, allogeneic, or unrelated).

In some embodiments, at least one or more chemotherapeutic agents may be administered in addition to the pharmaceutical composition comprising an immunogenic therapy.

In some embodiments, the one or more chemotherapeutic agents may belong to different classes of chemotherapeutic agents.

In practicing the methods of treatment or use provided herein, therapeutically-effective amounts of the pharmaceutical compositions can be administered to a subject having a disease or condition. A therapeutically-effective amount can vary widely depending on the severity of the disease, the age and relative health of the subject, the potency of the compounds used, and other factors.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (e.g., a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In some embodiments, the disease or condition that can be treated with the methods disclosed herein is abnormal growth of cells. In some embodiments, the disease or condition that can be treated with the methods disclosed herein is cancer. In some embodiments, the cancer is a malignant cancer. In some embodiments, the cancer is a benign cancer. In some embodiments, the cancer is an invasive cancer. In some embodiments, the cancer is a solid tumor. In some embodiments, the cancer is a liquid cancer.

The methods of the disclosure can be used to treat any type of cancer known in the art. Non-limiting examples of cancers to be treated by the methods of the present disclosure include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, RAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as RAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypemephroma, fibrosarcoma, transitional cell cancer (renal pelvis and/or ureter); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas.

In some embodiments, a subject with a mutation in a RAS gene is treated for cancer by administering a pharmaceutical composition comprising a TCR that recognizes a RAS mutant epitope in complex with an MHC protein encoded by the HLA allele of the subject, the TCR having a TCR alpha chain variable region and a TCR beta chain variable region having an amino acid sequence disclosed herein. In some embodiments, the pharmaceutical composition comprises a nucleic acid sequence encoding the TCR. In some embodiments, the nucleic acid is a DNA or an RNA. In some embodiments, the nucleic acid is a messenger RNA encoding the TCR having a TCR alpha chain variable region and a TCR beta chain variable region having an amino acid sequence disclosed herein. In some embodiments, the pharmaceutical composition comprises a vector that comprises a nucleic acid sequence encoding the TCR and is capable of driving the expression of the TCR, wherein the TCR recognizes a RAS mutant epitope in complex with an MHC protein encoded by the HLA allele of the subject, and wherein the TCR having a TCR alpha chain variable region and a TCR beta chain variable region having an amino acid sequence disclosed herein. In some embodiments, the pharmaceutical composition comprises a cell that comprises a nucleic acid sequence encoding the TCR that recognizes a RAS mutant epitope in complex with an MHC protein encoded by the HLA allele of the subject, the TCR having a TCR alpha chain variable region and a TCR beta chain variable region having an amino acid sequence disclosed herein. In some embodiments, the subject having a cancer is administered a pharmaceutical composition comprising a TCR that recognizes a RAS mutant epitope in complex with an MHC protein encoded by the HLA allele of the subject, the TCR having a TCR alpha chain variable region and a TCR beta chain variable region having an amino acid sequence disclosed herein, wherein the cancer is selected from adenocarcinoma of the biliary tract, transitional cell carcinoma of the bladder, breast carcinoma, cervical adenocarcinoma, colon adenocarcinoma, colon adenoma, neuroblastoma (autonomic ganglia), acute myeloid leukemia, chronic myeloid leukemia, chronic myelomonocytic leukemia, juvenile myelomonocytic leukemia, acute lymphoblastic leukemia, Burkitt's lymphoma, Hodgkin's lymphoma, plasma cell myeloma, hepatocellular carcinoma, large cell carcinoma, non-small cell carcinoma, ductal carcinoma, endocrine tumor, prostrate adenocarcinoma, basal cell carcinoma, squamous cell carcinoma, malignant melanoma, angiosarcoma, leiomyosarcoma, liposarcoma, rhabdomyosarcoma, myxoma, malignant fibrous histiocytoma, pleomorphic sarcoma, germinoma, seminoma, anaplastic carcinoma, follicular carcinoma, papillary carcinoma and Hurthle cell carcinoma.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Workflow for Antigen-Specific T Cell Identification and Validation

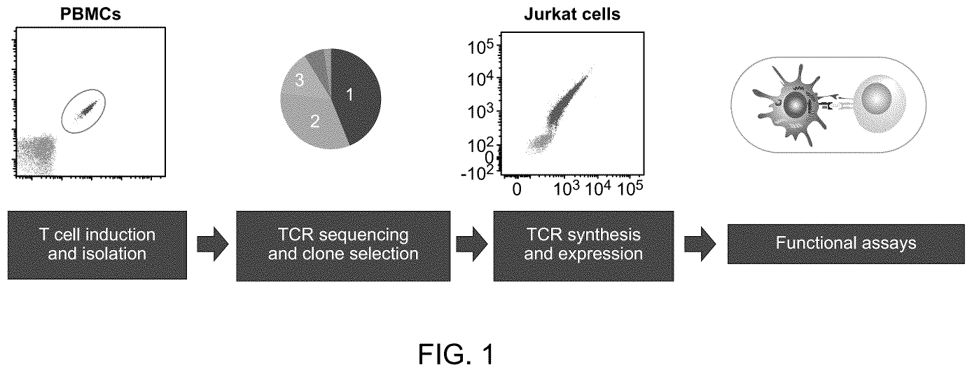

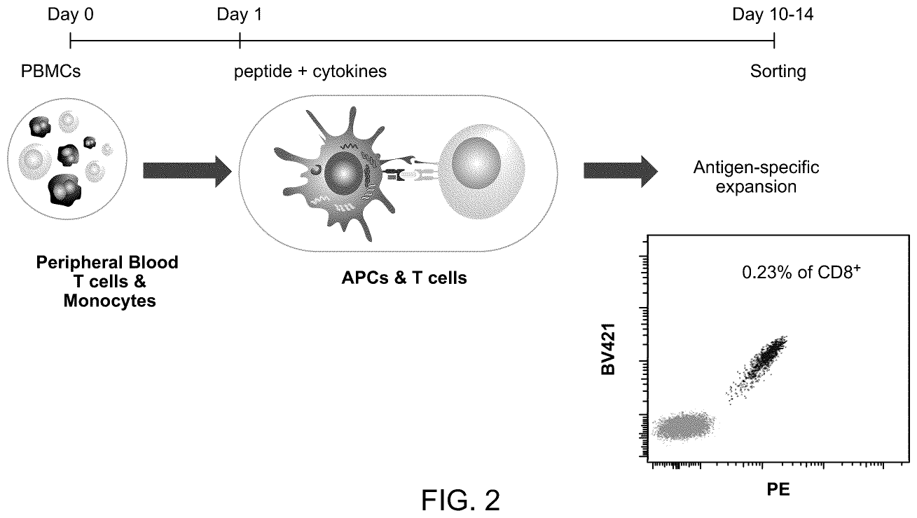

In this example, an exemplary workflow to generate and use antigen peptide specific TCRs is described. FIG. 1 demonstrates a graphical representation of the workflow. FIG. 2 details an exemplary timeline for the workflow process, starting with obtaining and culturing PBMCs from a subject, the PBMCs are then stabilized in overnight culture, and incubated in the presence of peptides and cytokines; the monocyte- and DC-derived antigen presenting cells (APCs) help induce T cells in response to the peptide antigens presented by the APCs; between 10 days to 2 weeks, antigen specific T cells expand and are sorted for antigen specificity and activation (for example, CD8+ marker expression indicating generation of cytotoxic T lymphocytes). The activated antigen responsive T cells contain antigen specific TCRs.

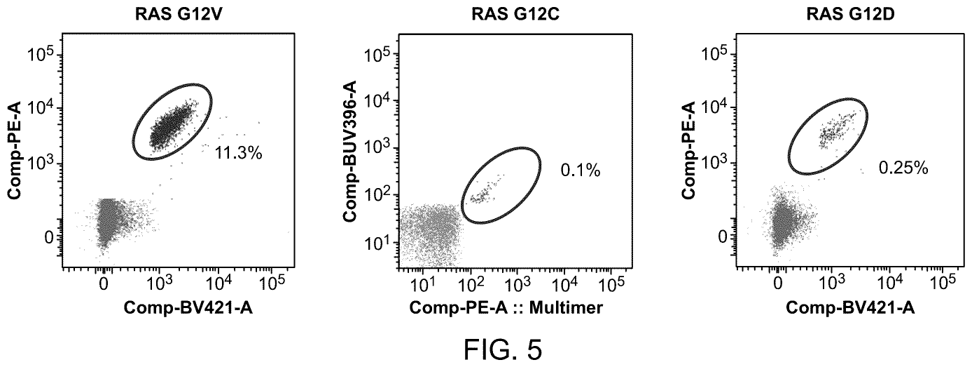

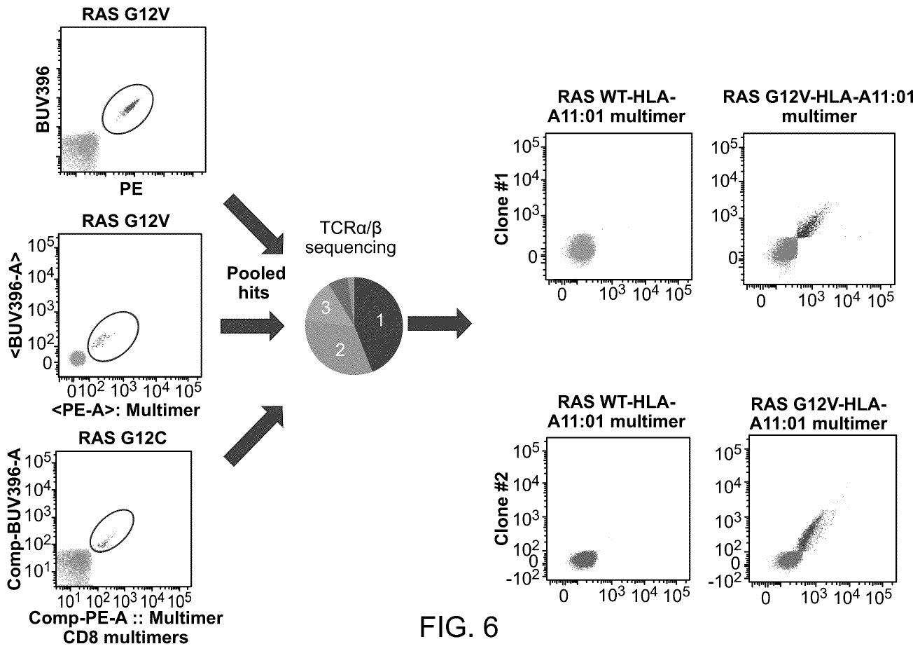

Briefly, Mutated peptides containing mutant target epitopes (e.g., mutant RAS peptides) were synthesized using a peptide synthesizer. The synthesized peptides were used to load APCs to stimulate T cells from a sample of healthy donor peripheral blood mononuclear cells (PBMCs). PBMCs from patients harboring neoantigens of interest can also be used to obtain neoantigen-specific T cells. After incubation with the peptide loaded APCs, T cell populations were analyzed by flow cytometry. Antigen-specific T cells were isolated using flow cytometry (FIGS. 1, 2, 4A, 5, and 6). Single cell TCR sequencing was performed for the isolated antigen-specific T cells using 10× Genomics Single Cell V(D)J system to profile the sequences of isolated antigen-specific TCRs. Sequencing reads were analyzed using the Cell Ranger™ analysis pipeline, and candidate sequences of TCRs were selected for further analysis and functional assays (FIGS. 1 and 6). Candidate sequences of TCRs can also be removed if the sequence of the beta chain CDR3 sequence comprises any one of SEQ ID NOs: 46-68 before the candidate is subjected to any further analysis and functional assays as described herein. For example, if the candidate TCR comprises a beta chain CDR3 sequence of any one of SEQ ID NOs: 46-68, the candidate TCR can be removed without being subjected to any further analysis or functional assays such that the eventually identified TCR would not comprise a beta chain CDR3 sequence of any one of SEQ ID NOs: 46-68.

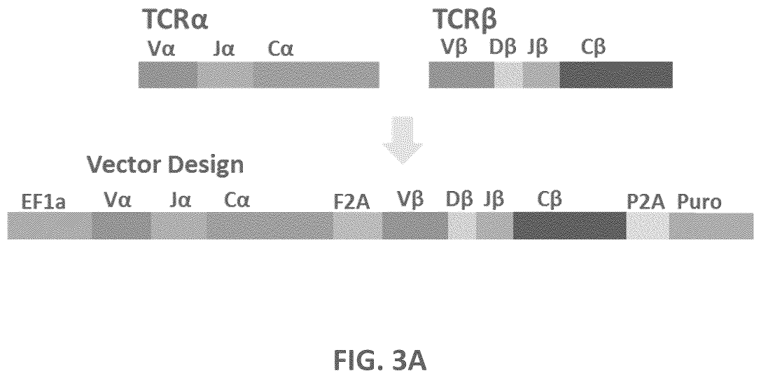

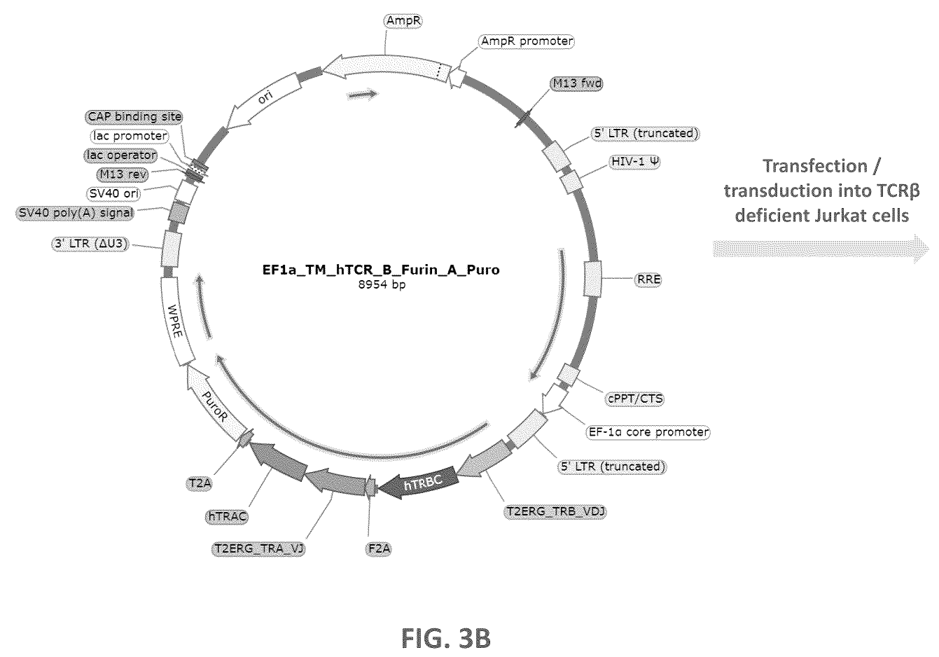

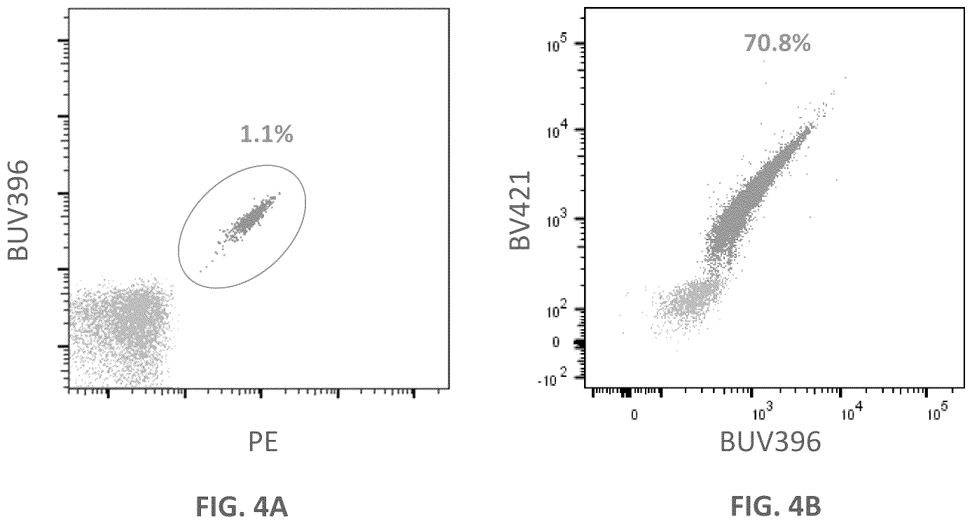

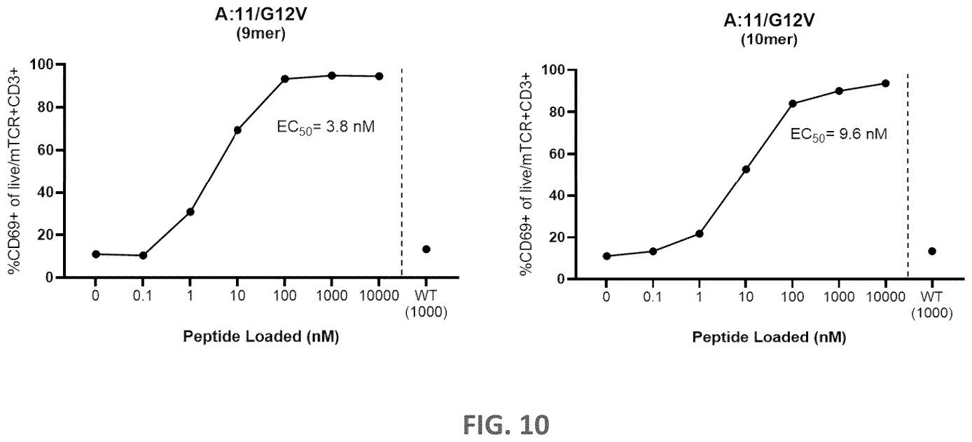

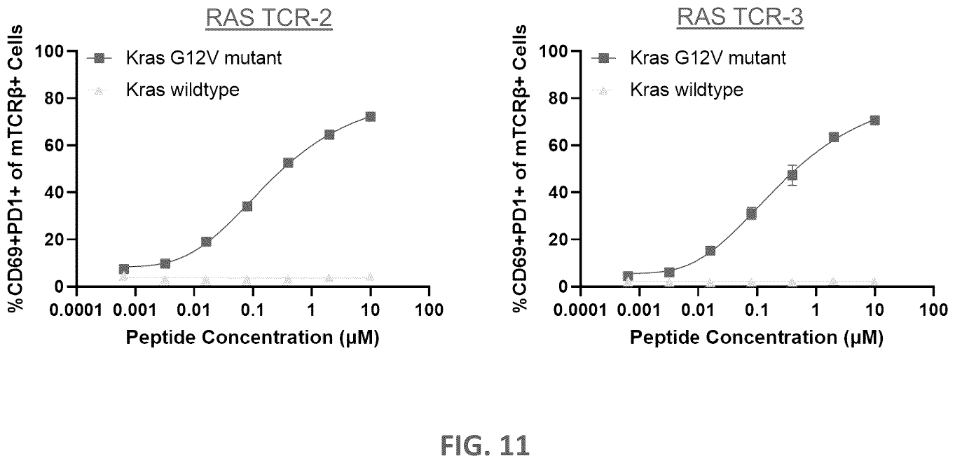

For analysis of TCR functionality, vectors or mRNA encoding RAS peptides containing target mutant epitopes were transduced into HEK293T or A375 cells to produce antigen expressing cell lines. Jurkat cells, TCRβ deficient Jurkat cells or PBMCs from healthy donors were transduced with nucleic acids (FIG. 3A) encoding candidate TCRs in a lentiviral vector. An exemplary vector is shown in FIG. 3B. FIGS. 4B, and 6 show functional assays for TCR-expressing vector transduced cells. RAS antigen expressing cell lines are cocultured with TCR transduced Jurkat cells, TCRβ deficient Jurkat cells or PBMCs from healthy donors for antigen recognition assays to analyze the functionality of candidate TCRs (FIG. 10 or 11).

### Example 2: Obtaining Antigen Specific T Cells

In vitro T cell inductions were used to expand antigen specific T cells. The healthy human donor PBMCs were seeded in each well of 24 well plate in AIM V media (Invitrogen). Mutant RAS peptides, TNF-α, IL-1β, PGE1, and IL-7 were added into wells after 24 hours incubation. The culture media was exchanged every 2 days with fresh media. The antigen specific T cells were evaluated and isolated at day 10 to 20 (FIG. 2).

### Example 3: HLA-Multimer Staining and Sorting

Neoantigen specific T cells were detected by combining HLA-multimer staining with 2 different fluorochrome conjugated recombinant HLA (e.g., HLA-A11.01 or HLA-C01.02) multimer with neoantigen peptides. Anti-CD8, anti-CD4, anti-CD19, anti-CD16, anti-CD14, anti-CD56 and antibodies and Live/Dead IR dye (Invitrogen) were used for cell surface staining. CD8⁺ T cells were identified as CD8⁺CD4⁻CD19⁻CD16⁻CD14⁻CD56⁻IR⁻ (FIGS. 1, 2, 4A, 5, and 6). For sorting, up to 5×10⁶ cells were incubated with 1-20 μg multimer in 100 μL PBS+0.5% human serum. Antibodies and Live/Dead IR dye were used to stain the cells for an additional 30 min. After staining, cells were washed twice and diluted in PBS+0.5% human serum. Live/Dead IR dye-negative cells were gated and CD8⁺/multimer⁺ T cells were sorted on a FACSAria cell sorter (BD Biosciences).

### Example 4: Deep Sequencing of Antigen-Specific TCRs

Antigen-specific T cells were sorted into a mixture of PBS and 2% FBS (Hyclone Defined), and 10× Genomics V(D)J kit was used to genetically barcode the RNAs from single T cells. RNA libraries were prepared according to the manufacturer's protocols. The resulting libraries were sequenced using ILLUMINA's MiSeq platform. 10× Genomics analysis software was then used to analyze the sequencing data to obtain paired TCR alpha and beta sequences. Dominant clones were identified based on the frequency of cells with a common TCR alpha and/or TCR beta (FIGS. 1, and 6).

### Example 5: Antigen-Specific TCR Gene Synthesis and Cloning

FIG. 3A represents a graphical diagram of an exemplary vector design, incorporating a TCR alpha chain and a TCR beta chain construct; wherein the TCR alpha- and beta-chain constructs comprise nucleic acid sequences encoding a variable (V), a diversity ((D), only in beta chain construct) a joining (J) and a constant (C) region for each of a TCR alpha (α) chain and a TCR beta (β) chain as shown in the diagram, in a vector with an upstream regulatory element comprising elements necessary for expression of the incorporated nucleic acid sequence, including, for example, a promoter (example EF1a region as shown in the diagram), further incorporating F2A and P2A proteolytic cleavage sites and a sequence encoding for Puromycin resistance (Puro) for Puromycin mediated selection. The lentiviral vectors were constructed from pCDH-EF1-T2A-Puro (SBI system bioscience). Antigen-specific TCR lentiviral vectors were generated by inserting a TCR beta variable region, followed by a TCR beta mouse constant region, furin cleavage site, SGSG linker (SEQ ID NO: 136), F2A site, a TCR alpha variable region, a TCR alpha constant region, a T2A site, and a puromycin resistance gene (FIGS. 3A and 3B).

### Example 6: Transfection and Transduction

Lentivirus encoding antigen-specific TCRs was prepared by transient transfection of 293T cells. The cells were seeded on a 10 cm plate at 7×10⁶ cells 16 hours before transfection with 7 μg of lentiviral vector, 7 μg of packaging plasmid mix (pPAX and pMD2.G), 28 μL of Fugene (Promega) and 1 mL of Opti-MEM (Gibco). The culture media was replaced a day after transfection. 72 hours after transfection, the supernatant was harvested concentrated 10-fold.

Jurkat cells or PBMCs were transduced with concentrated lentivirus encoding TCR sequences. Jurkat cells were washed and resuspended in RPMI-1640 containing polybrene and 10% FBS. 5×10⁵ CD8-Jurkat cells in 100 μL of media were plated per well in 96-well plate and L of concentrated lentivirus was added. The cells were centrifuged at 2400 rotation per minute (rpm) for 1 hour and incubated in a CO₂ incubator. The cells were transduced again with fresh media with polybrene and FBS and 25 μL of concentrated lentivirus. The media was replaced with RPMI-1640 containing 10% FBS and Pen/Strep 24 hours after the second transduction. Puromycin treatment (1 μg/ml) started at day 4 after transduction.

### Example 7: TCR Transduced Jurkat Binding to HLA-Peptide

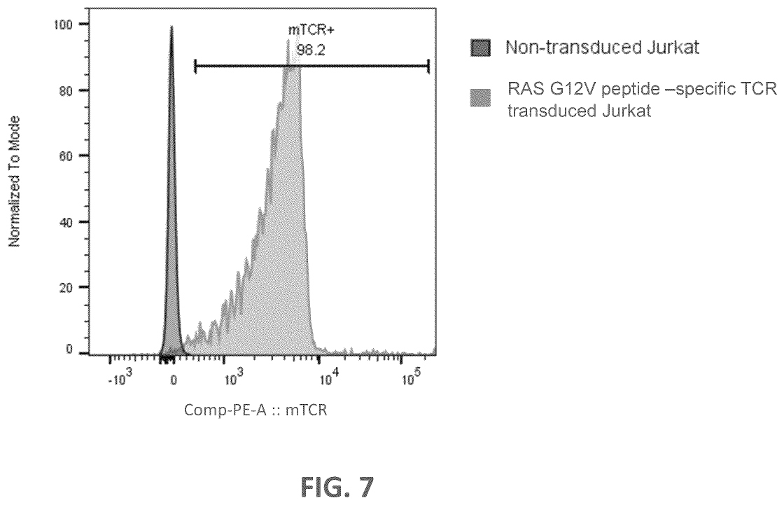

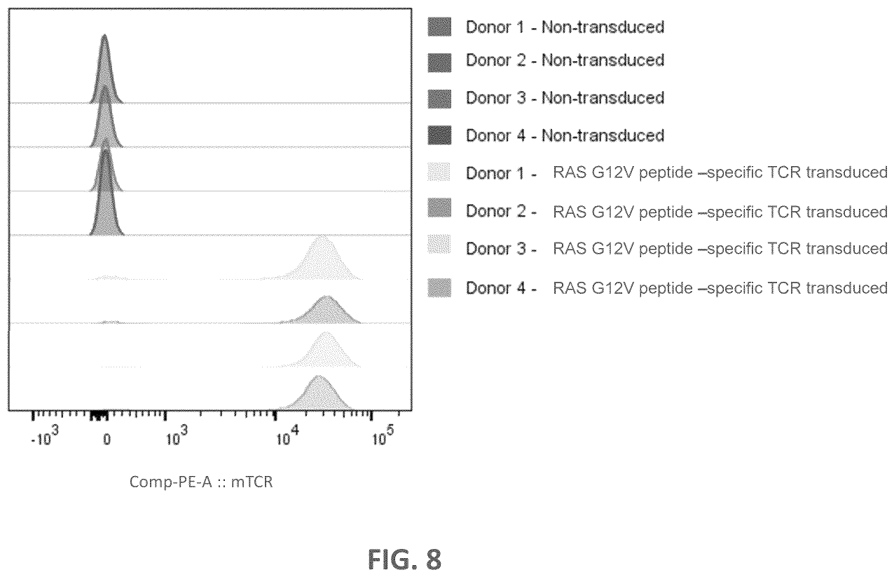

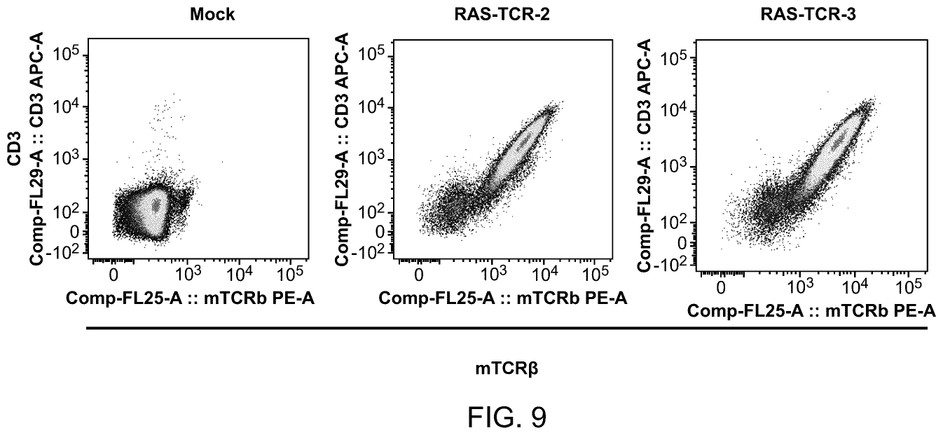

To evaluate TCR transduction, transduced Jurkat cells or PBMCs were stained with fluorochrome conjugated multimer (HLA-neoantigen), anti-CD8 antibody, anti-mTCR constant region antibody, and Live/Dead IR dye. mTCR and multimer positive cells were measured by flow cytometry (FIGS. 4B, and 6-8). FIG. 7 shows that Jurkat cells transduced with a RAS G12V peptide-specific TCR specifically expressed the TCR (i.e., RAS-TCR-1), relative to the non-transduced Jurkat cells. FIG. 8 shows that four different PBMC lines, each transduced with the RAS G12V peptide-specific TCR, specifically expressed the TCR, relative to the non-transduced PBMC lines. FIG. 9 shows that Jurkat cells electroporated with another two RAS G12V peptide-specific TCRs (i.e., RAS-TCR-2 and RAS-TCR-3) specifically expressed the TCRs, relative to the non-electroporated Jurkat cells.

### Example 8: Peptide Loading of Target Cells and Functional Avidity of RAS Mutant Peptide Specific TCRs

For exogenous peptide pulsing, 1×10⁶ T2, 293T or A375 cells were incubated at 37° C. and 5% CO₂ for 2 h with 10 pg/mL human β₂-microglobulin (Calbiochem) and titrating amounts, ranging from 1×10⁻⁵ M to 1×10⁻¹² M of the RAS peptides, T2 cells pulsed with 10⁻⁵ M influenza peptide GIL (influenza matrix protein₅₈₋₆₆ GILGFVTL (SEQ ID NO: 137), Metabion) served as negative control. After washing, peptide-loaded T2 cells were used in the TCR activation assay.

As shown in FIG. 10, for investigation of avidity and specificity of the RAS mutant peptide specific TCRs, Jurkat cells, transduced with and expressing a TCR specific to the mutant RAS peptide, were co-cultured with HLA-A11:01+ target cells pulsed with either the RAS G12V 9mer (VVGAVGVGK (SEQ ID NO: 43); left panel), RAS G12V 10mer (VVVGAVGVGK (SEQ ID NO: 44); right panel), or RAS WT peptide (data points on the far right side of both panels). CD69 was measured as a marker of activation (shown as a percentage of CD69+ cells relative to live TCR+ and CD3+ cells in the Y-axis of FIG. 10). Only the mutant G12V but not the WT RAS peptides could induce any activation of the transduced Jurkat cells, showing the specificity of the TCR to the mutant RAS peptide. Further, the avidity of the 9mer and 10mer were found to be 3.8 nM and 9.6 nM, respectively.

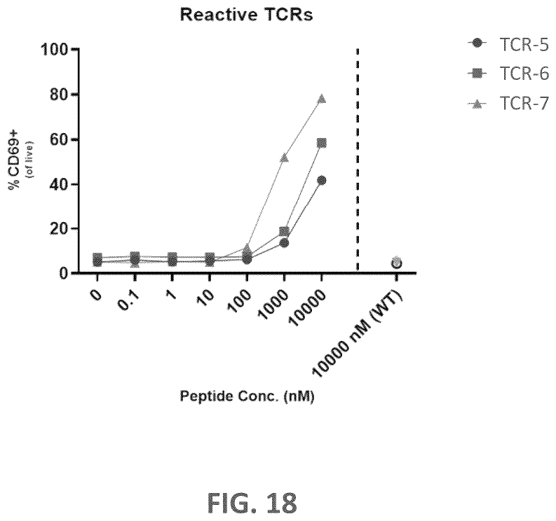

Likewise, as shown in FIG. 18, Jurkat cells, transduced with and expressing a TCR specific to the mutant RAS peptide (TCR-5, TCR-6, TCR-7), where co-cultured with HLA-C05:01+ target cells pulsed with Ras G12D 10mer (GADGVGKSAL (SEQ ID NO: 132)) or WT peptide. CD69 was measured as a marker of activation (shown as a percentage of CD69+ cells relative to total live cells in the Y-axis of FIG. 18). Results showed that the TCRs are reactive.

Furthermore, as shown in FIG. 11, for investigation of avidity and specificity of the other RAS mutant peptide specific TCRs, Jurkat cells, each electroporated with and expressing a TCR specific to the mutant RAS peptide (RAS-TCR-2 and RAS-TCR-3 on left and right panel, respectively), were co-cultured with HLA-C01:02+ target cells pulsed with either the RAS G12V mutant peptide (AVGVGKSAL (SEQ ID NO: 45)) or RAS WT peptide (data points on the far right side of both panels). CD69 and PD1 was measured as a marker of activation (shown as a percentage of CD69+ and PD1+ cells relative to live TCR+ and CD3+ cells in the Y-axis of FIG. 11). Only the mutant G12V but not the WT RAS peptides could induce any activation of the transduced Jurkat cells, showing the specificity of the TCRs to the mutant RAS peptide.

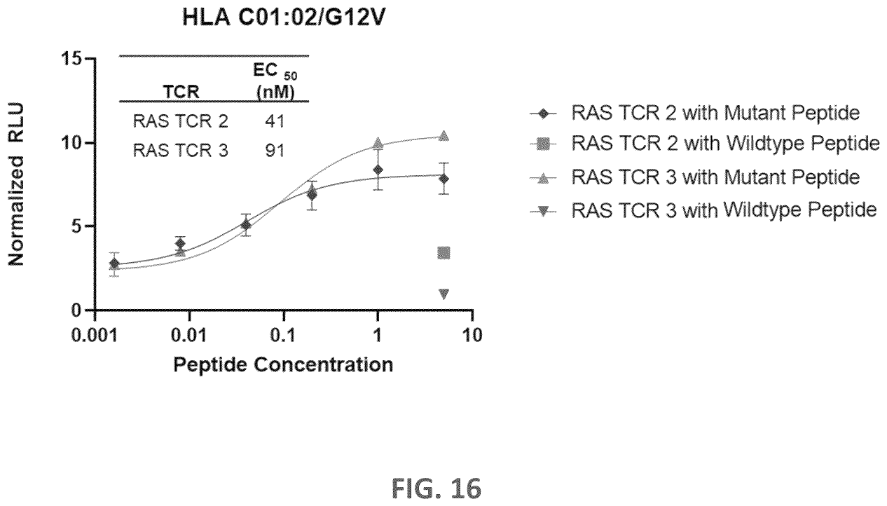

FIG. 16 depicts exemplary data showing functional avidity of RAS TCR-2 and RAS TCR-3. Transduced Jurkat NFAT luciferase reporter cells expressing either RAS TCR-2 or RAS TCR-3 were co-cultured with HLA-C*01:02+ target cells pulsed with either KRAS G12V 9mer or KRAS WT peptide. Relative Luciferase Unit was measured as a marker of T cell activation. Both RAS TCR-2 and RAS TCR-3 were specific and showed dose dependent activation.

### Example 9: Neoantigen Expressing Target Cells

For endogenous antigen expressing target cells, 1×10⁶ HEK293 or A375 cells were transduced with lentivirus vectors encoding RAS mutated peptides. The transduced target cells were selected by puromycin (1 μg/ml) in culture media (DMEM with 10% FBS).

### Example 10: IL-2 Release Assay

For investigation of specificity, T cell clones (2.5×10⁵ cells in 50 μL) can be incubated with HEK293T, HLA-A03.01 transduced A375 or HLA-A11.01 transduced A375 cell lines (5×10⁴ cells in 50 μL) and RAS mutated peptide neoantigens. Culture supernatants are harvested after 24 h co-culture and assessed for IL-2 concentration by a standard MSD using V-PLEX Human IL-2 assays (Meso Scale Discovery).

### Example 11: IFN-γ Release Assay

For investigation of specificity, T cell clones (2×10³ cells in 100 μL) are incubated with cell lines expressing RAS mutated peptide neoantigens. Culture supernatants are harvested after 24 h co-culture and assessed for the secretion of IFN-γ by a standard ELISA using the OptEIA™ Human IFN-γ Set (BD Biosciences Pharmingen).

### Example 12: Cytotoxicity Assay

Cytotoxic activity of T cell clones can be analyzed in a standard 4 h 51-chromium release assay. Briefly, 1×10⁶ target cells can be labeled with 100 μCi Na₂⁵¹CrO₄(ICN Biochemicals) for 1-1.5 h. ⁵¹Cr-labeled target cells can be cultured with T cells in RPMI 1640 with 12% FCS. For determination of functional avidity 1×10⁴ T cells can be added to 1×10³ peptide-pulsed T2 cells loaded with titrated amounts of RAS mutated peptide neoantigens, giving a constant E:T of 10:1.

After 4 h co-culture at 370 C, 50 μL of supernatant can be collected and radioactivity can be measured in a gamma counter. The percentage of specific lysis can be calculated as: 100×(experimental release-spontaneous release)/(maximum release−spontaneous release). Spontaneous release can be assessed by incubating target cells in the absence of effector cells. For the calculation of percent relative lysis, the maximum percent specific lysis can be set to the reference value of 100% and corresponding values can be calculated corresponding to this reference. To determine half-maximum lysis, percent relative lysis can be plotted against peptide concentration. The peptide concentration at which the curve crossed 50% relative lysis can be taken as the value of half-maximum lysis.

### Example 13: Cell Killing Assay

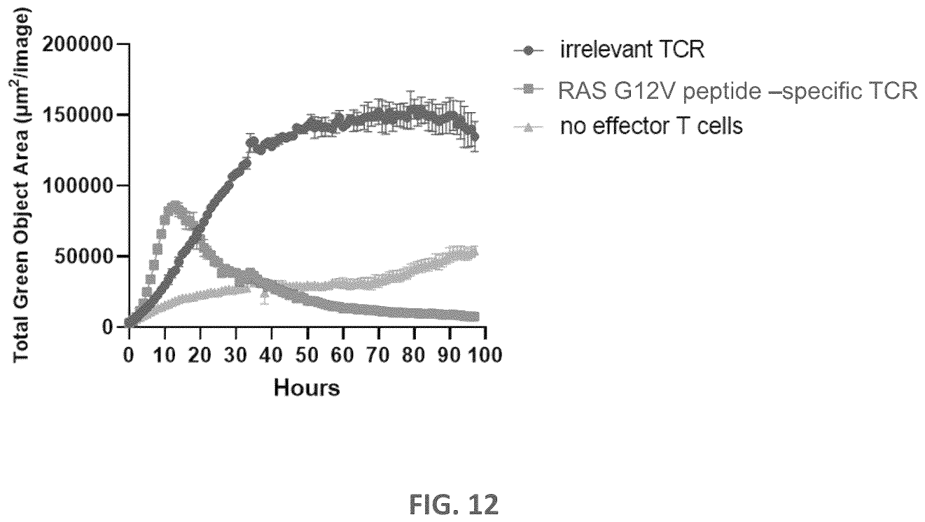

A RAS-neoantigen specific recombinant TCR was transduced into PBMCs and their ability to kill a cancer cell line was analyzed (FIG. 12). The recombinant TCR (rTCR) expressing cells showed significantly higher killing of cancer cell lines relative to controls. The SW620 cell line was used as a target cell which naturally expresses the KRAS G12V mutation. HLA-A11:01 was introduced into the SW620 cell line by lentiviral transductions. The rTCR transduced PBMCs were co-cultured with two different SW620 cell lines, with or without expression of HLA-A11:01, for 100 hours. Supernatant and cells were then harvested at various time points to analyze cytokine secretion (data not shown) and the growth of the SW620 cells.

Cytotoxicity activity was assessed by co-culturing the cells expressing a TCR specific to the mutant RAS peptide on a specific HLA, with the mutant RAS peptide-transduced target cancer cells expressing the corresponding HLA, and by determining the relative growth of the target cells, along with measuring the apoptotic marker Annexin V in the target cancer cells specifically (FIG. 12). The target cancer SW620 cells were engineered to express the mutant peptide along with the proper MHC-I allele. SW620 target tumor cells cultured alone or co-cultured with PBMCs transduced with an irrelevant TCR were used as the negative controls. Mock-transduced target cells (e.g., not expressing the mutant peptide) can also be used as a negative control. The cells were also transduced to stably express GFP allowing the tracking of target cell growth. PBMCs from healthy donors, used as effector cells, were transduced to express a TCR specific to a mutant RAS peptide. The target cells were cocultured with the effector cells in a 10:1 ratio for 100 hours in media containing Annexin V-detection reagent. The GFP signal and Annexin-V signal were measured over time with an IncuCyte S3 apparatus. Annexin V signal originating from effector cells was filtered out by size exclusion. Target cell growth and death was expressed as GFP and Annexin-V area (mm²) over time, respectively (Y-axis of FIG. 12).

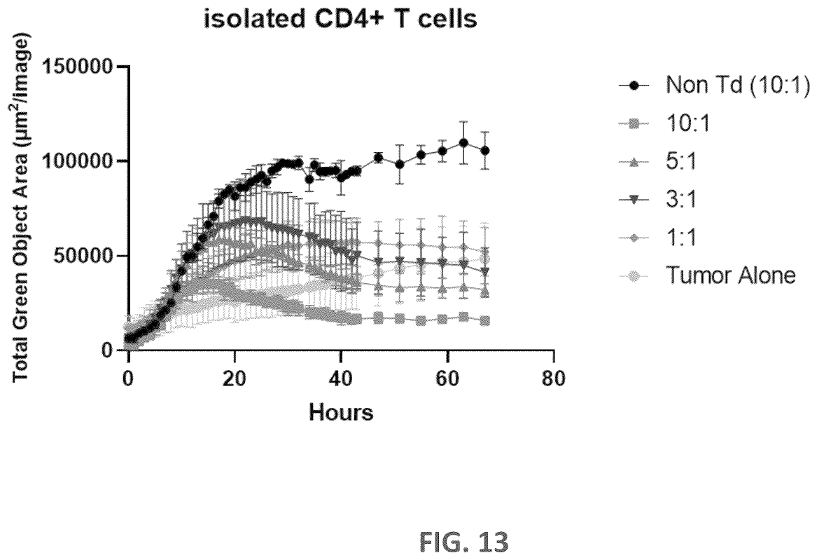

Specific killing of the target cell was also observed when T cells were used in the killing assays: Cytotoxicity activity was assessed by co-culturing CD4+ T cells, isolated from PBMCs expressing the TCR specific to the mutant RAS peptide on a specific HLA, with mutant RAS peptide-transduced target cancer cells expressing the corresponding HLA, and by determining the relative growth of the target cells, along with measuring the apoptotic marker Annexin V in the target cancer cells specifically (FIG. 13). Target cancer SW620 cells were engineered to express the mutant peptide along with the proper MHC-I allele. SW620 target tumor cells cultured alone or co-cultured with CD4+ T cells isolated from PBMCs not transduced with RAS mutant peptide-specific TCR were used as the negative controls. Mock-transduced target cells (e.g., not expressing the mutant peptide) can also be used as a negative control. The target cells were cocultured with the effector cells in 10:1, 5:1, 3:1 and 1:1 ratio for 100 hours in media containing Annexin V-detection reagent. As shown in FIG. 13, higher amounts of CD4+ T cells relative to the target cells induced increasingly amount of cell killings in the target cells expressing the mutant RAS peptide. Because the killing of the target cell was induced by isolated CD4+ T cells co-cultured with the target cancer cells, the data also suggests that the killing is independent from CD8 proteins.

In some examples, after co-culturing, significantly higher levels of IFNγ, IL-2, and TNFα can be detected for the groups of HLA-A11:01 or HLA-C01:02 transduced SW620 cell line (Ras^{mut} cell line+HLA-A11:01 or HLA-C01:02) compared to non-transduced SW620 (Ras^{mut} cell line). Also, a significantly higher percent of Caspase-3 (apoptosis marker) positive cells can be observed in HLA-A11:01 or HLA-C01:02 transduced SW620 cell lines compared to control. Thus, the rTCR transduced PBMCs can not only secrete cytokine (IFNγ, IL-2, and TNFα) but also functionally kill target cells specifically.

In some examples, cytotoxic activity is assessed before TCR cloning. T cells induced against a mutant RAS peptide on a specific HLA are co-cultured with mutant target cancer cells expressing the corresponding HLA loaded with a range of concentrations of the RAS mutant peptides. The relative growth and the apoptotic marker Annexin V in the target cancer cells specifically are both measured. The target cells are also transduced to stably express GFP allowing the tracking of target cell growth. The GFP signal and Annexin-V signal are measured over time with an IncuCyte S3 apparatus. Annexin V signal originating from effector cells is filtered out by size exclusion. Target cell growth and death is expressed as GFP and Annexin-V area (mm²) over time, respectively. For T cells demonstrating cytotoxicity at low peptide concentrations compared to no peptide and/or wild-type peptide, TCR sequencing can be performed as above.

### Example 14A: SNG-M Cell Killing Assay

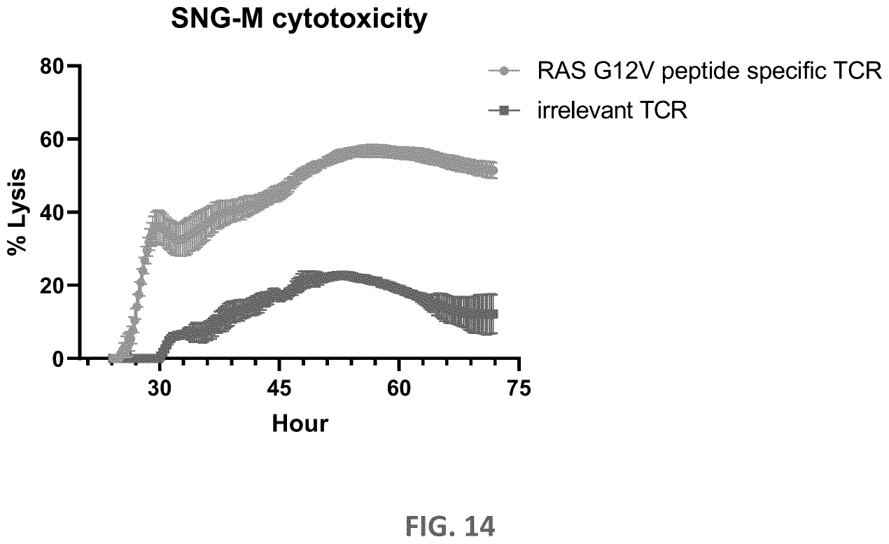

The tumor cell line, SNG-M, endogenously expresses HLA-A11:01 and a low level of KRAS G12V. Healthy donor peripheral blood mononuclear cells were transduced to express RAS TCR-1. Irrelevant TCR transduced T cells were used as a negative transduced T cell control. TCR transduced T cells were co-cultured with SNG-M tumor cells at an effector to target cell ratio of 3:1. Killing was measured via impedance on the xCELLigence RTCA MP. % lysis was calculated using SNG-M tumor cells cultured in the absence of any effector T cells. RAS TCR-1 transduced T cells resulted in ˜3× killing of SNG-M over time compared to irrelevant TCR transduced T cells (FIG. 14).

### Example 14B: Engineered SW-60 Cell Killing Assay

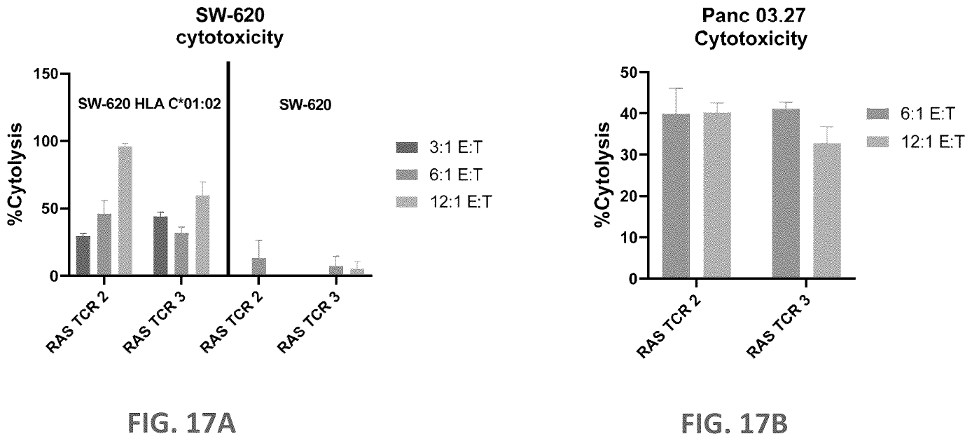

The colorectal cancer cell line, SW-620, endogenously harbors KRAS G12V mutation. SW-620 cells were engineered to express HLA C*01:02, the cognate HLA of RAS TCR-2 and RAS TCR-3. Healthy donor peripheral blood mononuclear cells were transduced to express either RAS TCR-2 or RAS TCR-3. Non transduced T cells were used as a negative control. TCR transduced T cells were co-cultured with either SW-620 or SW-620 HLA C*01:02 cells at various effector to target cell ratios (3:1, 6:1 and 12:1). Killing was measured via impedance on the xCELLigence RTCA MP. % Cytolysis was calculated based on tumor cells cocultured with non-transduced T cells. Both RAS TCR-2 and RAS TCR-3 transduced T cells can effectively recognize and kill SW-620 HLA C*01:02 based on endogenous antigen expression (FIG. 17A).

### Example 14C: Panc 03.27 Cell Killing Assay

The pancreatic tumor cell line, Panc 03.27, endogenously expresses HLA-C01:02 and KRAS G12V mutation. Healthy donor peripheral blood mononuclear cells were transduced to express either RAS TCR-2 or RAS TCR-3. Non transduced T cells were used as a negative control. TCR transduced T cells were co-cultured with Panc 03.27 tumor cells at two different effector to target cell ratios (6:1 and 12:1). Killing was measured via impedance on the xCELLigence platform. % Cytolysis was calculated based on tumor cells cocultured with non-transduced T cells. Both RAS TCR-2 and RAS TCR-3 transduced T cells can effectively recognize and kill Panc 03.27 tumor cells based on endogenous HLA and antigen expression (FIG. 17B).

### Example 15: Tumor Regression Assay

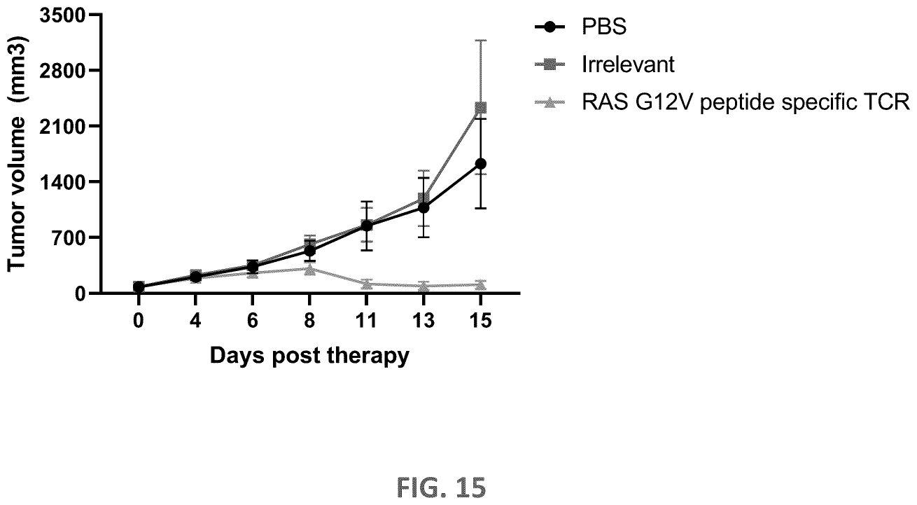

Immunodeficient NCG (PrkdcKO, IL2RgKO, and SirpaKO NOG) mice were subcutaneously inoculated with the tumor line A375-HLA-A11:01-KRAS G12V, which is an A375 tumor line engineered to express HLA-A11:01 and KRAS G12V. Healthy donor peripheral blood mononuclear cells were transduced to express RAS TCR-1. Healthy donor peripheral blood mononuclear cells were also transduced with an irrelevant chimeric antigen receptor as a negative transduced T cell control. When tumors reached an average of 80 mm\hat{ }3, PBS or effector T cells were injected into the mice intravenously. Mice were treated with 6e6 antigen specific TCR transduced T cells, for the irrelevant transduced T cell group, a total cell dose was matched. Mice treated with RAS TCR-1 transduced T cells exhibited tumor regression compared to the PBS and irrelevant transduced T cell controls (FIG. 15).

### Example 16: Summary of Recombinant TCRs Developed

This example provides the details of the TCRs developed. Table 1 provides a synopsis of the TCRs described herein and Table 2 provides the specific amino acid and coding sequences.

TABLE 1

| | | | | | | | Method of | |
|---|---|---|---|---|---|---|---|---|
| | | | | | | | determining | |
| | | | | | | | TCR binding | |
| | | | | | | | to peptide- | |
| | | | | | | TCR | MHC | |
| | | | | | | binding | | IL-2 by |
| | | | | SEQ | | to | | Jurkat |
| TCR | | | | ID | TCRbeta | peptide- | Multimer | and/or |
| name | Gene | Mutation | Allele | NO: | CDR3 | MHC | Hit | PBMC |
| RAS | RAS | G12V | A11: | 6 | CASSVD | — | — | — |
| TCR-1 | | | 01 | | SSHNE | | | |
| | | | | | QFF | | | |
| RAS | RAS | G12V | C01: | 20 | CASSRS | — | — | — |
| TCR-2 | | | 02 | | WPPGY | | | |
| | | | | | TF | | | |
| RAS | RAS | G12V | C01: | 34 | CASRD | — | — | — |
| TCR-3 | | | 02 | | MTGKA | | | |
| | | | | | FF | | | |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 2

| | SEQ ID | |
|---|---|---|
| Name | NO | Sequence |
| RAS TCR- | 1 | DRVSQS |
| 1 alpha | | |
| chain | | |
| CDR1 | | |
| RAS TCR- | 2 | IYSNGD |
| 1 alpha | | |
| chain | | |
| CDR2 | | |
| RAS TCR- | 3 | CAVRDSGAGSYQLTF |
| 1 alpha | | |
| chain | | |
| CDR3 | | |
| RAS TCR- | 4 | SGDLS |
| 1 beta | | |
| chain | | |
| CDR1 | | |
| RAS TCR- | 5 | YYNGEE |
| 1 beta | | |
| chain | | |
| CDR2 | | |
| RAS TCR- | 6 | CASSVDSSHNEQFF |
| 1 beta | | |
| chain | | |
| CDR3 | | |
| RAS TCR- | 7 | atgaaatccttgagagttttactagtgatcctgtggcttcagttg |
| 1 alpha | | agctgggtttggagccaacagaaggaggtggagcagaattctgga |
| chain | | cccctcagtgttccagagggagccattgcctctctcaactgcact |
| variable | | tacagtgaccgagtttcccagtccttcttctggtacagacaatat |
| domain | | tctgggaaaagccctgagttgataatgtccatatactccaatggt |
| | | gacaaagaagatggaaggtttacagcacagctcaataaagccagc |
| | | cagtatgtttctctgctcatcagagactcccagcccagtgattca |
| | | gccacctacctctgtgccgtgagggactctggggctgggagttac |
| | | caactcactttcgggaaggggaccaaactctcggtcatacca |
| | 8 | atgaagtctctgagggtgctgctggtcatcctgtggctgcagctg |
| | | tcttgggtgtggagccagcagaaggaggtggagcagaactccgga |
| | | ccactgtctgtgcctgagggagccatcgccagcctgaattgcaca |
| | | tactccgacagagtgagccagtccttcttttggtacaggcagtat |
| | | tccggcaagtctcccgagctgatcatgagcatctactccaacggc |
| | | gacaaggaggatggcagattcaccgcccagctgaataaggccagc |
| | | cagtacgtgagcctgctgatccgcgactcccagccctctgatagc |
| | | gccacatacctgtgcgccgtgcgggattctggagcaggcagctat |
| | | cagctgacctttggcaagggcacaaagctgagcgtgatccct |
| | 9 | MKSLRVLLVILWLQLSWVWSQQKEVEQNSGPLSVPEGAIASLNCT |
| | | YSDRVSQSFFWYRQYSGKSPELIMSIYSNGDKEDGRFTAQLNKAS |
| | | QYVSLLIRDSQPSDSATYLCAVRDSGAGSYQLTFGKGTKLSVIP |
| RAS TCR- | 10 | atgggcttcaggctcctctgctgtgtggccttttgtctcctggga |
| 1 beta | | gcaggcccagtggattctggagtcacacaaaccccaaagcacctg |
| chain | | atcacagcaactggacagcgagtgacgctgagatgctcccctagg |
| variable | | tctggagacctctctgtgtactggtaccaacagagcctggaccag |
| domain | | ggcctccagttcctcattcagtattataatggagaagagagagca |
| | | aaaggaaacattcttgaacgattctccgcacaacagttccctgac |
| | | ttgcactctgaactaaacctgagctctctggagctgggggactca |
| | | gctttgtatttctgtgccagcagcgtagattcctcccacaatgag |
| | | cagttcttcgggccagggacacggctcaccgtgcta |
| | 11 | atgggcttccggctgctgtgctgcgtggcattttgcctgctggga |
| | | gcaggaccagtggactccggcgtgacccagacacccaagcacctg |
| | | atcaccgcaacaggacagagggtgaccctgagatgttcccctagg |
| | | tctggcgacctgagcgtgtactggtatcagcagagcctggatcag |
| | | ggcctgcagttcctgatccagtactataacggcgaggagagagcc |
| | | aagggcaatatcctggagaggttcagcgcccagcagtttccagac |
| | | ctgcactccgagctgaatctgagctccctggagctgggcgacagc |
| | | gccctgtacttctgcgcctctagcgtggattcctctcacaacgag |
| | | cagttctttggaccaggaaccaggctgacagtgctg |
| | 12 | MGFRLLCCVAFCLLGAGPVDSGVTQTPKHLITATGQRVTLRCSPR |
| | | SGDLSVYWYQQSLDQGLQFLIQYYNGEERAKGNILERFSAQQFPD |
| | | LHSELNLSSLELGDSALYFCASSVDSSHNEQFFGPGTRLTVL |
| RAS TCR- | 13 | MKSLRVLLVILWLQLSWVWSQQKEVEQNSGPLSVPEGAIASLNCT |
| 1 alpha | | YSDRVSQSFFWYRQYSGKSPELIMSIYSNGDKEDGRFTAQLNKAS |
| chain | | QYVSLLIRDSQPSDSATYLCAVRDSGAGSYQLTFGKGTKLSVIPI |
| (TRAV12- | | QNPDPAVYQLRDSKSSDKSVCLFTDFDSQTNVSQSKDSDVYITDK |
| 2 TRAJ28 | | TVLDMRSMDFKSNSAVAWSNKSDFACANAFNNSIIPEDTFFPSPE |
| human | | SSCDVKLVEKSFETDTNLNFQNLSVIGFRILLLKVAGFNLLMTLR |
| TRAC) | | LWSS |
| RAS TCR- | 69 | MKSLRVLLVILWLQLSWVWSQQKEVEQNSGPLSVPEGAIASLNCT |
| 1 alpha | | YSDRVSQSFFWYRQYSGKSPELIMSIYSNGDKEDGRFTAQLNKAS |
| chain | | QYVSLLIRDSQPSDSATYLCAVRDSGAGSYQLTFGKGTKLSVIPD |
| (TRAV12- | | IQNPEPAVYQLKDPRSQDSTLCLFTDFDSQINVPKTMESGTFITD |
| 2 TRAJ28 | | KTVLDMKAMDSKSNGAIAWSNQTSFTCQDIFKETNATYPSSDVPC |
| mouse | | DATLTEKSFETDMNLNFQNLSVMGLRILLLKVAGFNLLMTLRLWS |
| TRAC) | | S |
| RAS TCR- | 75 | Atgaagtctctgagggtgctgctggtcatcctgtggctgcagctg |
| 1 alpha | | tcttgggtgtggagccagcagaaggaggtggagcagaactccgga |
| chain | | ccactgtctgtgcctgagggagccatcgccagcctgaattgcaca |
| (TRAV12- | | tactccgacagagtgagccagtccttcttttggtacaggcagtat |
| 2 TRAJ28 | | tccggcaagtctcccgagctgatcatgagcatctactccaacggc |
| mouse | | gacaaggaggatggcagattcaccgcccagctgaataaggccagc |
| TRAC) | | cagtacgtgagcctgctgatccgcgactcccagccctctgatagc |
| | | gccacatacctgtgcgccgtgcgggattctggagcaggcagctat |
| | | cagctgacctttggcaagggcacaaagctgagcgtgatccctgat |
| | | atccaaaaccctgagcccgccgtgtaccagctgaaagatcctaga |
| | | tcccaggactccacactgtgcctctttaccgatttcgacagccag |
| | | atcaatgtgcccaagaccatggagagcggcaccttcatcaccgat |
| | | aagacagtgctcgatatgaaagccatggacagcaagtccaatggc |
| | | gccattgcttggagcaaccagacctccttcacctgccaggacatt |
| | | tttaaagagaccaacgccacctaccccagcagcgacgtgccttgt |
| | | gatgccaccctgaccgagaagagcttcgaaaccgacatgaacctg |
| | | aactttcagaacctgagcgtcatgggactcagaatcctcctgctc |
| | | aaagtcgccggcttcaacctcctgatgaccctgagactgtggagc |
| | | tcc |
| RAS TCR- | 14 | MGFRLLCCVAFCLLGAGPVDSGVTQTPKHLITATGQRVTLRCSPR |
| 1 beta | | SGDLSVYWYQQSLDQGLQFLIQYYNGEERAKGNILERFSAQQFPD |
| chain | | LHSELNLSSLELGDSALYFCASSVDSSHNEQFFGPGTRLTVLEDL |
| (TRBV9 | | KNVFPPEVAVFEPSEAEISHTQKATLVCLATGFYPDHVELSWWVN |
| TRBJ2-1 | | GKEVHSGVSTDPQPLKEQPALNDSRYCLSSRLRVSATFWQNPRNH |
| human | | FRCQVQFYGLSENDEWTQDRAKPVTQIVSAEAWGRADCGFTSESY |
| TRBC2) | | QQGVLSATILYEILLGKATLYAVLVSALVLMAMVKRKDSRG |
| RAS TCR- | 70 | MGFRLLCCVAFCLLGAGPVDSGVTQTPKHLITATGQRVTLRCSPR |
| 1 beta | | SGDLSVYWYQQSLDQGLQFLIQYYNGEERAKGNILERFSAQQFPD |
| chain | | LHSELNLSSLELGDSALYFCASSVDSSHNEQFFGPGTRLTVLKDL |
| (TRBV9 | | RNVTPPKVSLFEPSKAEIANKQKATLVCLARGFFPDHVELSWWVN |
| TRBJ2-1 | | GKEVHSGVSTDPQAYKESNYSYCLSSRLRVSATFWHNPRNHFRCQ |
| mouse | | VQFHGLSEEDKWPEGSPKPVTQNISAEAWGRADCGITSASYHQGV |
| TRBC2) | | LSATILYEILLGKATLYAVLVSGLVLMAMVKKKNS |
| RAS TCR- | 76 | atgggcttccggctgctgtgctgcgtggcattttgcctgctggga |
| 1 beta | | gcaggaccagtggactccggcgtgacccagacacccaagcacctg |
| chain | | atcaccgcaacaggacagagggtgaccctgagatgttcccctagg |
| (TRBV9 | | tctggcgacctgagcgtgtactggtatcagcagagcctggatcag |
| TRBJ2-1 | | ggcctgcagttcctgatccagtactataacggcgaggagagagcc |
| mouse | | aagggcaatatcctggagaggttcagcgcccagcagtttccagac |
| TRBC2) | | ctgcactccgagctgaatctgagctccctggagctgggcgacagc |
| | | gccctgtacttctgcgcctctagcgtggattcctctcacaacgag |
| | | cagttctttggaccaggaaccaggctgacagtgctgaaggatctg |
| | | cggaatgtgaccccccctaaggtgtccctgtttgagccctctaag |
| | | gccgagatcgccaacaagcagaaggccaccctggtgtgcctggca |
| | | aggggcttctttcctgaccacgtggagctgtcctggtgggtgaat |
| | | ggcaaggaggtgcacagcggcgtgtccaccgatccacaggcctac |
| | | aaggagtctaactacagctattgcctgagctccaggctgcgcgtg |
| | | agcgccacattctggcacaaccctcgcaatcacttccggtgtcag |
| | | gtgcagtttcacggcctgtctgaggaggacaagtggccagagggc |
| | | agcccaaagccagtgacccagaatatctccgccgaggcatgggga |
| | | agggcagattgcggcatcacatctgccagctatcaccagggcgtg |
| | | ctgagcgccaccatcctgtacgagatcctgctgggcaaggccaca |
| | | ctgtatgccgtgctggtgtctggcctggtgctgatggccatggtg |
| | | aagaagaagaacagc |
| RAS TCR- | 15 | SIFNT |
| 2 alpha | | |
| chain | | |
| CDR1 | | |
| RAS TCR- | 16 | LYKAGEL |
| 2 alpha | | |
| chain | | |
| CDR2 | | |
| RAS TCR- | 17 | CAGQLEGDDKIIF |
| 2 alpha | | |
| chain | | |
| CDR3 | | |
| RAS TCR- | 18 | KGHSH |
| 2 beta | | |
| chain | | |
| CDR1 | | |
| RAS TCR- | 19 | LQKENI |
| 2 beta | | |
| chain | | |
| CDR2 | | |
| RAS TCR- | 20 | CASSRSWPPGYTF |
| 2 beta | | |
| chain | | |
| CDR3 | | |
| RAS TCR- | 21 | atgctccttgaacatttattaataatcttgtggatgcagctgaca |
| 2 alpha | | tgggtcagtggtcaacagctgaatcagagtcctcaatctatgttt |
| chain | | atccaggaaggagaagatgtctccatgaactgcacttcttcaagc |
| variable | | atatttaacacctggctatggtacaagcaggaccctggggaaggt |
| domain | | cctgtcctcttgatagccttatataaggctggtgaattgacctca |
| | | aatggaagactgactgctcagtttggtataaccagaaaggacagc |
| | | ttcctgaatatctcagcatccatacctagtgatgtaggcatctac |
| | | ttctgtgctgggcagctagagggggatgacaagatcatctttgga |
| | | aaagggacacgacttcatattctcccc |
| | 22 | atgctccttgaacatttattaataatcttgtggatgcagctgaca |
| | | tgggtcagtggtcaacagctgaatcagagtcctcaatctatgttt |
| | | atacaagagggagaagatgtctccatgaactgcacttcttcaagc |
| | | atatttaacacctggctatggtacaagcaggaccctggggaaggt |
| | | cctgtcctcttgattgccttatataaggctggtgaattgacctca |
| | | aatggaagactgactgctcagtttggtataaccagaaaggacagc |
| | | ttcctgaatatctcagcatccatacctagtgatgtaggcatctac |
| | | ttctgtgctggtcaactagagggggatgacaagatcatctttgga |
| | | aaagggacacgacttcatattctcccc |
| | 23 | MLLEHLLIILWMQLTWVSGQQLNQSPQSMFIQEGEDVSMNCTSSS |
| | | IFNTWLWYKQDPGEGPVLLIALYKAGELTSNGRLTAQFGITRKDS |
| | | FLNISASIPSDVGIYFCAGQLEGDDKIIFGKGTRLHILP |
| RAS TCR- | 24 | atggacaccagactactctgctgtgcggtcatctgtcttctgggg |
| 2 beta | | gcaggtctctcaaatgccggcgtcatgcagaacccaagacacctg |
| chain | | gtcaggaggaggggacaggaggcaagactgagatgcagcccaatg |
| variable | | aaaggacacagtcatgtttactggtatcggcagctcccagaggaa |
| domain | | ggtctgaaattcatggtttatctccagaaagaaaatatcatagat |
| | | gagtcaggaatgccaaaggaacgattttctgctgaatttcccaaa |
| | | gagggccccagcatcctgaggatccagcaggtagtgcgaggagat |
| | | tcggcagcttatttctgtgccagctcacgctcttggccccctggc |
| | | tacaccttcggttcggggaccaggttaaccgttgta |
| | 25 | atggacaccagactactctgctgtgcggtcatctgtcttctgggg |
| | | gcaggtctctcaaatgccggcgtcatgcagaacccaagacacctg |
| | | gtcaggaggaggggacaggaggcaagactgagatgcagcccaatg |
| | | aaaggacacagtcatgtttactggtataggcaattaccagaggaa |
| | | ggtctgaaattcatggtttatttacaaaaggaaaatatcatagat |
| | | gagtcaggaatgccaaaggaacgattttctgctgaatttcccaaa |
| | | gagggccccagcatcctgaggatccagcaagtcgtgcgaggagat |
| | | tcggcggcttatttctgtgcgtcttctcgctcttggccccctggc |
| | | tacaccttcggttcggggaccaggttaaccgttgta |
| | 26 | MDTRLLCCAVICLLGAGLSNAGVMQNPRHLVRRRGQEARLRCSPM |
| | | KGHSHVYWYRQLPEEGLKFMVYLQKENIIDESGMPKERFSAEFPK |
| | | EGPSILRIQQVVRGDSAAYFCASSRSWPPGYTFGSGTRLTVV |
| RAS TCR- | 27 | MLLEHLLIILWMQLTWVSGQQLNQSPQSMFIQEGEDVSMNCTSSS |
| 2 alpha | | IFNTWLWYKQDPGEGPVLLIALYKAGELTSNGRLTAQFGITRKDS |
| chain | | FLNISASIPSDVGIYFCAGQLEGDDKIIFGKGTRLHILPIQNPDP |
| (TRAV35 | | AVYQLRDSKSSDKSVCLFTDFDSQTNVSQSKDSDVYITDKTVLDM |
| TRAJ30 | | RSMDFKSNSAVAWSNKSDFACANAFNNSIIPEDTFFPSPESSCDV |
| human | | KLVEKSFETDTNLNFQNLSVIGFRILLLKVAGFNLLMTLRLWSS |
| TRAC) | | |
| RAS TCR- | 71 | METLLGVSLVILWLQLARVNGQQLNQSPQSMFIQEGEDVSMNCTS |
| 2 alpha | | SSIFNTWLWYKQDPGEGPVLLIALYKAGELTSNGRLTAQFGITRK |
| chain | | DSFLNISASIPSDVGIYFCAGQLEGDDKIIFGKGTRLHILPIQNP |
| (TRAV35 | | EPAVYQLKDPRSQDSTLCLFTDFDSQINVPKTMESGTFITDKCVL |
| TRAJ30 | | DMKAMDSKSNGAIAWSNQTSFTCQDIFKETNATYPSSDVPCDATL |
| mouse | | TEKSFETDMNLNFQNLLVIVLRILLLKVAGFNLLMTLRLWSS |
| TRAC) | | |
| RAS TCR- | 28 | MDTRLLCCAVICLLGAGLSNAGVMQNPRHLVRRRGQEARLRCSPM |
| 2 beta | | KGHSHVYWYRQLPEEGLKFMVYLQKENIIDESGMPKERFSAEFPK |
| chain | | EGPSILRIQQVVRGDSAAYFCASSRSWPPGYTFGSGTRLTVVEDL |
| (TRBV18 | | NKVFPPEVAVFEPSEAEISHTQKATLVCLATGFFPDHVELSWWVN |
| TRBJ1-2 | | GKEVHSGVSTDPQPLKEQPALNDSRYCLSSRLRVSATFWQNPRNH |
| human | | FRCQVQFYGLSENDEWTQDRAKPVTQIVSAEAWGRADCGFTSVSY |
| TRBC1) | | QQGVLSATILYEILLGKATLYAVLVSALVLMAMVKRKDF |
| RAS TCR- | 72 | MGTRLLCWAALCLLGAELTNAGVMQNPRHLVRRRGQEARLRCSPM |
| 2 beta | | KGHSHVYWYRQLPEEGLKFMVYLQKENIIDESGMPKERFSAEFPK |
| chain | | EGPSILRIQQVVRGDSAAYFCASSRSWPPGYTFGSGTRLTVVEDL |
| (TRBV18 | | RNVTPPKVSLFEPSKAEIANKQKATLVCLARGFFPDHVELSWWVN |
| TRBJ1-2 | | GKEVHSGVCTDPQAYKESNYSYCLSSRLRVSATFWHNPRNHFRCQ |
| mouse | | VQFHGLSEEDKWPEGSPKPVTQNISAEAWGRADCGITSASYQQGV |
| TRBC1) | | LSATILYEILLGKATLYAVLVSTLVVMAMVKRKNS |
| RAS TCR- | 29 | TSESDYY |
| 3 alpha | | |
| chain | | |
| CDR1 | | |
| RAS TCR- | 30 | QEAYKQQN |
| 3 alpha | | |
| chain | | |
| CDR2 | | |
| RAS TCR- | 31 | CAYWGWETSGSRLTF |
| 3 alpha | | |
| chain | | |
| CDR3 | | |
| RAS TCR- | 32 | MNHEY |
| 3 beta | | |
| chain | | |
| CDR1 | | |
| RAS TCR- | 33 | SVGAGI |
| 3 beta | | |
| chain | | |
| CDR2 | | |
| RAS TCR- | 34 | CASRDMTGKAFF |
| 3 beta | | |
| chain | | |
| CDR3 | | |
| RAS TCR- | 35 | atggcatgccctggcttcctgtgggcacttgtgatctccacctgt |
| 3 alpha | | cttgaatttagcatggctcagacagtcactcagtctcaaccagag |
| chain | | atgtctgtgcaggaggcagagaccgtgaccctgagctgcacatat |
| variable | | gacaccagtgagagtgattattatttattctggtacaagcagcct |
| domain | | cccagcaggcagatgattctcgttattcgccaagaagcttataag |
| | | caacagaatgcaacagagaatcgtttctctgtgaacttccagaaa |
| | | gcagccaaatccttcagtctcaagatctcagactcacagctgggg |
| | | gatgccgcgatgtatttctgtgcttattggggttgggaaaccagt |
| | | ggctctaggttgacctttggggaaggaacacagctcacagtgaat |
| | | cct |
| | 36 | atggcatgccctggcttcctgtgggcacttgtgatctccacctgt |
| | | cttgaatttagcatggctcagacagtcactcagtctcaaccagag |
| | | atgtctgtgcaggaggcagagaccgtgaccctgagctgcacatat |
| | | gacaccagtgagagtgattattatttattctggtacaagcagcct |
| | | cccagcaggcagatgattctcgttattcgccaagaagcttataag |
| | | caacagaatgcaacagagaatcgtttctctgtgaacttccaaaag |
| | | gcagccaaatccttctcgctcaagatctcagactcacagctgggg |
| | | gatgccgcgatgtatttctgtgcttattggggttgggaaaccagt |
| | | ggctctaggttgacctttggggaaggaacacagctcacagtgaat |
| | | cct |
| | 37 | MACPGFLWALVISTCLEFSMAQTVTQSQPEMSVQEAETVTLSCTY |
| | | DTSESDYYLFWYKQPPSRQMILVIRQEAYKQQNATENRFSVNFQK |
| | | AAKSFSLKISDSQLGDAAMYFCAYWGWETSGSRLTFGEGTQLTVN |
| | | P |
| RAS TCR- | 38 | atgagcatcggcctcctgtgctgtgcagccttgtctctcctgtgg |
| 3 beta | | gcaggtccagtgaatgctggtgtcactcagaccccaaaattccag |
| chain | | gtcctgaagacaggacagagcatgacactgcagtgtgcccaggat |
| variable | | atgaaccatgaatacatgtcctggtatcgacaagacccaggcatg |
| domain | | gggctgaggctgattcattactcagttggtgctggtatcactgac |
| | | caaggagaagtccccaatggctacaatgtctccagatcaaccaca |
| | | gaggatttcccgctcaggctgctgtcggctgctccctcccagaca |
| | | tctgtgtacttctgtgccagcagggatatgacagggaaggctttc |
| | | tttggacaaggcaccagactcacagttgta |
| | 39 | atgagcatcggcctcctgtgctgtgcagccttgtctctcctgtgg |
| | | gcaggtccagtgaatgctggtgtcactcagaccccaaaattccag |
| | | gtcctgaagacaggacagagcatgacactgcagtgtgcccaggat |
| | | atgaaccatgaatacatgtcctggtatcgacaagacccaggcatg |
| | | gggctgaggctgattcattactcagttggtgccggtatcactgac |
| | | caaggagaagtccccaatggctacaatgtctcgcgttctacgact |
| | | gaagatttcccgctcaggctgctgtcggctgctcccagtcaaacg |
| | | tctgtgtacttctgtgcgtctagagatatgacagggaaggctttc |
| | | tttggacaaggcaccagactcacagttgta |
| | 40 | MSIGLLCCAALSLLWAGPVNAGVTQTPKFQVLKTGQSMTLQCAQD |
| | | MNHEYMSWYRQDPGMGLRLIHYSVGAGITDQGEVPNGYNVSRSTT |
| | | EDFPLRLLSAAPSQTSVYFCASRDMTGKAFFGQGTRLTVV |
| RAS TCR- | 41 | MACPGFLWALVISTCLEFSMAQTVTQSQPEMSVQEAETVTLSCTY |
| 3 alpha | | DTSESDYYLFWYKQPPSRQMILVIRQEAYKQQNATENRFSVNFQK |
| chain | | AAKSFSLKISDSQLGDAAMYFCAYWGWETSGSRLTFGEGTQLTVN |
| (TRAV38- | | PIQNPDPAVYQLRDSKSSDKSVCLFTDFDSQTNVSQSKDSDVYIT |
| 2/DV8 | | DKTVLDMRSMDFKSNSAVAWSNKSDFACANAFNNSIIPEDTFFPS |
| TRAJ58 | | PESSCDVKLVEKSFETDTNLNFQNLSVIGFRILLLKVAGFNLLMT |
| human | | LRLWSS |
| TRAC) | | |
| RAS TCR- | 73 | METLLGVSLVILWLQLARVNAQTVTQSQPEMSVQEAETVTLSCTY |
| 3 alpha | | DTSESDYYLFWYKQPPSRQMILVIRQEAYKQQNATENRFSVNFQK |
| chain | | AAKSFSLKISDSQLGDAAMYFCAYWGWETSGSRLTFGEGTQLTVN |
| (TRAV38- | | PIQNPEPAVYQLKDPRSQDSTLCLFTDFDSQINVPKTMESGTFIT |
| 2/DV8 | | DKCVLDMKAMDSKSNGAIAWSNQTSFTCQDIFKETNATYPSSDVP |
| TRAJ58 | | CDATLTEKSFETDMNLNFQNLLVIVLRILLLKVAGFNLLMTLRLW |
| mouse | | SS |
| TRAC) | | |
| RAS TCR- | 42 | MSIGLLCCAALSLLWAGPVNAGVTQTPKFQVLKTGQSMTLQCAQD |
| 3 beta | | MNHEYMSWYRQDPGMGLRLIHYSVGAGITDQGEVPNGYNVSRSTT |
| chain | | EDFPLRLLSAAPSQTSVYFCASRDMTGKAFFGQGTRLTVVEDLNK |
| (TRBV6-5 | | VFPPEVAVFEPSEAEISHTQKATLVCLATGFFPDHVELSWWVNGK |
| TRBJ-1 | | EVHSGVSTDPQPLKEQPALNDSRYCLSSRLRVSATFWQNPRNHFR |
| human | | CQVQFYGLSENDEWTQDRAKPVTQIVSAEAWGRADCGFTSVSYQQ |
| TRBC1) | | GVLSATILYEILLGKATLYAVLVSALVLMAMVKRKDF |
| RAS TCR- | 74 | MGTRLLCWAALCLLGAELTNAGVTQTPKFQVLKTGQSMTLQCAQD |
| 3 beta | | MNHEYMSWYRQDPGMGLRLIHYSVGAGITDQGEVPNGYNVSRSTT |
| chain | | EDFPLRLLSAAPSQTSVYFCASRDMTGKAFFGQGTRLTVVEDLRN |
| (TRBV6-5 | | VTPPKVSLFEPSKAEIANKQKATLVCLARGFFPDHVELSWWVNGK |
| TRBJ-1 | | EVHSGVCTDPQAYKESNYSYCLSSRLRVSATFWHNPRNHFRCQVQ |
| mouse | | FHGLSEEDKWPEGSPKPVTQNISAEAWGRADCGITSASYQQGVLS |
| TRBC1) | | ATILYEILLGKATLYAVLVSTLVVMAMVKRKNS |
| RAS TCR- | 77 | NYSPAY |
| 4 alpha | | |
| chain | | |
| CDR1 | | |
| RAS TCR- | 78 | IRENEKE |
| 4 alpha | | |
| chain | | |
| CDR2 | | |
| RAS TCR- | 79 | CALVSYGSGGSQGNLIF |
| 4 alpha | | |
| chain | | |
| CDR3 | | |
| RAS TCR- | 80 | SGHNS |
| 4 beta | | |
| chain | | |
| CDR1 | | |
| RAS TCR- | 81 | FNNNVP |
| 4 beta | | |
| chain | | |
| CDR2 | | |
| RAS TCR- | 82 | CASSLSLNTEAFF |
| 4 beta | | |
| chain | | |
| CDR3 | | |
| RAS TCR- | 83 | MAFWLRRLGLHFRPHLGRRMESFLGGVLLILWLQVDWVKSQKIEQ |
| 4 alpha | | NSEALNIQEGKTATLTCNYTNYSPAYLQWYRQDPGRGPVFLLLIR |
| chain | | ENEKEKRKERLKVTFDTTLKQSLFHITASQPADSATYLCALVSYG |
| variable | | SGGSQGNLIFGKGTKLSVKP |
| domain | | |
| RAS TCR- | 84 | MDSWTFCCVSLCILVAKHTDAGVIQSPRHEVTEMGQEVTLRCKPI |
| 4 beta | | SGHNSLFWYRQTMMRGLELLIYFNNNVPIDDSGMPEDRFSAKMPN |
| chain | | ASFSTLKIQPSEPRDSAVYFCASSLSLNTEAFFGQGTRLTVV |
| variable | | |
| domain | | |
| RAS TCR- | 85 | NSASQS |
| 5 alpha | | |
| chain | | |
| CDR1 | | |
| RAS TCR- | 86 | VYSSGN |
| 5 alpha | | |
| chain | | |
| CDR2 | | |
| RAS TCR- | 87 | CVVIPMDSSYKLIF |
| 5 alpha | | |
| chain | | |
| CDR3 | | |
| RAS TCR- | 88 | MDHEN |
| 5 beta | | |
| chain | | |
| CDR1 | | |
| RAS TCR- | 89 | SYDVKM |
| 5 beta | | |
| chain | | |
| CDR2 | | |
| RAS TCR- | 90 | CASTWGEGEAFF |
| 5 beta | | |
| chain | | |
| CDR3 | | |
| RAS TCR- | 91 | MISLRVLLVILWLQLSWVWSQRKEVEQDPGPFNVPEGATVAFNCT |
| 5 alpha | | YSNSASQSFFWYRQDCRKEPKLLMSVYSSGNEDGRFTAQLNRASQ |
| chain | | YISLLIRDSKLSDSATYLCVVIPMDSSYKLIFGSGTRLLVRP |
| variable | | |
| domain | | |
| RAS TCR- | 92 | MGIRLLCRVAFCFLAVGLVDVKVTQSSRYLVKRTGEKVFLECVQD |
| 5 beta | | MDHENMFWYRQDPGLGLRLIYFSYDVKMKEKGDIPEGYSVSREKK |
| chain | | ERFSLILESASTNQTSMYLCASTWGEGEAFFGQGTRLTVV |
| variable | | |
| domain | | |
| RAS TCR- | 15 | SIFNT |
| 6 alpha | | |
| chain | | |
| CDR1 | | |
| RAS TCR- | 16 | LYKAGEL |
| 6 alpha | | |
| chain | | |
| CDR2 | | |
| RAS TCR- | 93 | CAGQELIDGNQFYF |
| 6 alpha | | |
| chain | | |
| CDR3 | | |
| RAS TCR- | 94 | SGHDY |
| 6 beta | | |
| chain | | |
| CDR1 | | |
| RAS TCR- | 81 | FNNNVP |
| 6 beta | | |
| chain | | |
| CDR2 | | |
| RAS TCR- | 95 | CASSPGSSYEQYF |
| 6 beta | | |
| chain | | |
| CDR3 | | |
| RAS TCR- | 96 | MLLEHLLIILWMQLTWVSGQQLNQSPQSMFIQEGEDVSMNCTSSS |
| 6 alpha | | IFNTWLWYKQEPGEGPVLLIALYKAGELTSNGRLTAQFGITRKDS |
| chain | | FLNISASIPSDVGIYFCAGQELIDGNQFYFGTGTSLTVIP |
| variable | | |
| domain | | |
| RAS TCR- | 97 | MDSWTLCCVSLCILVAKHTDAGVIQSPRHEVTEMGQEVTLRCKPI |
| 6 beta | | SGHDYLFWYRQTMMRGLELLIYFNNNVPIDDSGMPEDRFSAKMPN |
| chain | | ASFSTLKIQPSEPRDSAVYFCASSPGSSYEQYFGPGTRLTVT |
| variable | | |
| domain | | |
| RAS TCR- | 98 | KALYS |
| 7 alpha | | |
| chain | | |
| CDR1 | | |
| RAS TCR- | 99 | LLKGGEQ |
| 7 alpha | | |
| chain | | |
| CDR2 | | |
| RAS TCR- | 100 | CGTEIRAGGGADGLTF |
| 7 alpha | | |
| chain | | |
| CDR3 | | |
| RAS TCR- | 94 | SGHDY |
| 7 beta | | |
| chain | | |
| CDR1 | | |
| RAS TCR- | 81 | FNNNVP |
| 7 beta | | |
| chain | | |
| CDR2 | | |
| RAS TCR- | 101 | CASSFFLGETQYF |
| 7 beta | | |
| chain | | |
| CDR3 | | |
| RAS TCR- | 102 | METLLKVLSGTLLWQLTWVRSQQPVQSPQAVILREGEDAVINCSS |
| 7 alpha | | SKALYSVHWYRQKHGEAPVFLMILLKGGEQKGHDKISASFNEKKQ |
| chain | | QSSLYLTASQLSYSGTYFCGTEIRAGGGADGLTFGKGTHLIIQP |
| variable | | |
| domain | | |
| RAS TCR- | 103 | MDSWTLCCVSLCILVAKHTDAGVIQSPRHEVTEMGQEVTLRCKPI |
| 7 beta | | SGHDYLFWYRQTMMRGLELLIYFNNNVPIDDSGMPEDRFSAKMPN |
| chain | | ASFSTLKIQPSEPRDSAVYFCASSFFLGETQYFGPGTRLLVL |
| variable | | |
| domain | | |
| RAS TCR- | 104 | NIATNDY |
| 8 alpha | | |
| chain | | |
| CDR1 | | |
| RAS TCR- | 105 | GYKTK |
| 8 alpha | | |
| chain | | |
| CDR2 | | |
| RAS TCR- | 106 | CLVGDGDNARLMF |
| 8 alpha | | |
| chain | | |
| CDR3 | | |
| RAS TCR- | 107 | SGHDT |
| 8 beta | | |
| chain | | |
| CDR1 | | |
| RAS TCR- | 108 | YYEEEE |
| 8 beta | | |
| chain | | |
| CDR2 | | |
| RAS TCR- | 109 | CASSLPRMGSDTEAFF |
| 8 beta | | |
| chain | | |
| CDR3 | | |
| RAS TCR- | 110 | MRQVARVIVFLTLSTLSLAKTTQPISMDSYEGQEVNITCSHNNIA |
| 8 alpha | | TNDYITWYQQFPSQGPRFIIQGYKTKVTNEVASLFIPADRKSSTL |
| chain | | SLPRVSLSDTAVYYCLVGDGDNARLMFGDGTQLVVKP |
| variable | | |
| domain | | |
| RAS TCR- | 111 | MGPGLLCWALLCLLGAGLVDAGVTQSPTHLIKTRGQQVTLRCSPK |
| 8 beta | | SGHDTVSWYQQALGQGPQFIFQYYEEEERQRGNFPDRFSGHQYPN |
| chain | | YSSELNVNALLLGDSALYLCASSLPRMGSDTEAFFGQGTRLTVV |
| variable | | |
| domain | | |
| RAS TCR- | 104 | NIATNDY |
| 9 alpha | | |
| chain | | |
| CDR1 | | |
| RAS TCR- | 105 | GYKTK |
| 9 alpha | | |
| chain | | |
| CDR2 | | |
| RAS TCR- | 112 | CLVGDMDGGYQKVTF |
| 9 alpha | | |
| chain | | |
| CDR3 | | |
| RAS TCR- | 88 | MDHEN |
| 9 beta | | |
| chain | | |
| CDR1 | | |
| RAS TCR- | 89 | SYDVKM |
| 9 beta | | |
| chain | | |
| CDR2 | | |
| RAS TCR- | 113 | CASSPTRLGYNEQFF |
| 9 beta | | |
| chain | | |
| CDR3 | | |
| RAS TCR- | 114 | MRQVARVIVFLTLSTLSLAKTTQPISMDSYEGQEVNITCSHNNIA |
| 9 alpha | | TNDYITWYQQFPSQGPRFIIQGYKTKVTNEVASLFIPADRKSSTL |
| chain | | SLPRVSLSDTAVYYCLVGDMDGGYQKVTFGTGTKLQVIP |
| variable | | |
| domain | | |
| RAS TCR- | 115 | MGIRLLCRVAFCFLAVGLVDVKVTQSSRYLVKRTGEKVFLECVQD |
| 9 beta | | MDHENMFWYRQDPGLGLRLIYFSYDVKMKEKGDIPEGYSVSREKK |
| chain | | ERFSLILESASTNQTSMYLCASSPTRLGYNEQFFGPGTRLTVL |
| variable | | |
| domain | | |
| RAS TCR- | 104 | NIATNDY |
| 10 alpha | | |
| chain | | |
| CDR1 | | |
| RAS TCR- | 105 | GYKTK |
| 10 alpha | | |
| chain | | |
| CDR2 | | |
| RAS TCR- | 116 | CLVGDLDGQKLLF |
| 10 alpha | | |
| chain | | |
| CDR3 | | |
| RAS TCR- | 107 | SGHDT |
| 10 beta | | |
| chain | | |
| CDR1 | | |
| RAS TCR- | 108 | YYEEEE |
| 10 beta | | |
| chain | | |
| CDR2 | | |
| RAS TCR- | 117 | CASSLRTGEAFF |
| 10 beta | | |
| chain | | |
| CDR3 | | |
| RAS TCR- | 118 | MRQVARVIVFLTLSTLSLAKTTQPISMDSYEGQEVNITCSHNNIA |
| 10 alpha | | TNDYITWYQQFPSQGPRFIIQGYKTKVTNEVASLFIPADRKSSTL |
| chain | | SLPRVSLSDTAVYYCLVGDLDGQKLLFARGTMLKVDL |
| variable | | |
| domain | | |
| RAS TCR- | 119 | MGPGLLCWALLCLLGAGLVDAGVTQSPTHLIKTRGQQVTLRCSPK |
| 10 beta | | SGHDTVSWYQQALGQGPQFIFQYYEEEERQRGNFPDRFSGHQFPN |
| chain | | YSSELNVNALLLGDSALYLCASSLRTGEAFFGQGTRLTVV |
| variable | | |
| domain | | |
| RAS TCR- | 104 | NIATNDY |
| 11 alpha | | |
| chain | | |
| CDR1 | | |
| RAS TCR- | 105 | GYKTK |
| 11 alpha | | |
| chain | | |
| CDR2 | | |
| RAS TCR- | 120 | CLVGDMDSGNTPLVF |
| 11 alpha | | |
| chain | | |
| CDR3 | | |
| RAS TCR- | 121 | LNHDA |
| 11 beta | | |
| chain | | |
| CDR1 | | |
| RAS TCR- | 122 | SQIVND |
| 11 beta | | |
| chain | | |
| CDR2 | | |
| RAS TCR- | 123 | CASSIDYRGIGETQYF |
| 11 beta | | |
| chain | | |
| CDR3 | | |
| RAS TCR- | 124 | MRQVARVIVFLTLSTLSLAKTTQPISMDSYEGQEVNITCSHNNIA |
| 11 alpha | | TNDYITWYQQFPSQGPRFIIQGYKTKVTNEVASLFIPADRKSSTL |
| chain | | SLPRVSLSDTAVYYCLVGDMDSGNTPLVFGKGTRLSVIA |
| variable | | |
| domain | | |
| RAS TCR- | 125 | MSNQVLCCVVLCLLGANTVDGGITQSPKYLFRKEGQNVTLSCEQN |
| 11 beta | | LNHDAMYWYRQDPGQGLRLIYYSQIVNDFQKGDIAEGYSVSREKK |
| chain | | ESFPLTVTSAQKNPTAFYLCASSIDYRGIGETQYFGPGTRLLVL |
| variable | | |
| domain | | |
| RAS TCR- | 29 | TSESDYY |
| 12 alpha | | |
| chain | | |
| CDR1 | | |
| RAS TCR- | 30 | QEAYKQQN |
| 12 alpha | | |
| chain | | |
| CDR2 | | |
| RAS TCR- | 126 | CAWEGGSEKLVF |
| 12 alpha | | |
| chain | | |
| CDR3 | | |
| RAS TCR- | 127 | LGHNA |
| 12 beta | | |
| chain | | |
| CDR1 | | |
| RAS TCR- | 128 | YNFKEQ |
| 12 beta | | |
| chain | | |
| CDR2 | | |
| RAS TCR- | 129 | CASSSSGGRIYGYTF |
| 12 beta | | |
| chain | | |
| CDR3 | | |
| RAS TCR- | 130 | METLLGVSLVILWLQLARVNAQTVTQSQPEMSVQEAETVTLSCTY |
| 12 alpha | | DTSESDYYLFWYKQPPSRQMILVIRQEAYKQQNATENRFSVNFQK |
| chain | | AAKSFSLKISDSQLGDAAMYFCAWEGGSEKLVFGKGTKLTVNP |
| variable | | |
| domain | | |
| RAS TCR- | 131 | MGTRLLCWAALCLLGAELTETGVTQTPRHLVMGMTNKKSLKCEQH |
| 12 beta | | LGHNAMYWYKQSAKKPLELMFVYNFKEQTENNSVPSRFSPECPNS |
| chain | | SHLFLHLHTLQPEDSALYLCASSSSGGRIYGYTFGSGTRLTVV |
| variable | | |
| domain | | |
| RAS G12V | 43 | VVGAVGVGK |
| 9mer | | |
| RAS G12V | 44 | VVVGAVGVGK |
| 10mer | | |
| RAS G12V | 45 | AVGVGKSAL |
| 9mer #2 | | |
| RAS G12D | 132 | GADGVGKSAL |
| RAS G12V | 133 | GAVGVGKSAL |
| 10mer #2 | | |
| RAS G12V | 134 | GAVGVGKSA |
| 9mer #3 | | |
| RAS G12D | 135 | VVVGADGVGK |
| #2 | | |
| other | 46 | SARDRGLVSLPSVEAFF |
| CDR3-1 | | |
| other | 47 | ASYLSGSIYNEQFF |
| CDR3-2 | | |
| other | 48 | ASSYSTERGTIY |
| CDR3-3 | | |
| other | 49 | ASSLADIYEQY |
| CDR3-4 | | |
| other | 50 | CASSARNDEAFF |
| CDR3-5 | | |
| other | 51 | CASSLGDSEQYF |
| CDR3-6 | | |
| other | 52 | CASSQRSNTGELFF |
| CDR3-7 | | |
| other | 53 | CASGGRDSTDTQYF |
| CDR3-8 | | |
| other | 54 | ASSTSFWEVNTEAF |
| CDR3-9 | | |
| other | 55 | ASSKRGWPYEQY |
| CDR3-10 | | |
| other | 56 | ASSLADIYEQY |
| CDR3-11 | | |
| other | 57 | ASSSTDRIEAF |
| CDR3-12 | | |
| other | 58 | ASTTFKTGRAIEKLF |
| CDR3-13 | | |
| other | 59 | ASSSRGHSGTEAF |
| CDR3-14 | | |
| other | 60 | ASSSRGHSGTEAF |
| CDR3-15 | | |
| other | 61 | ATYKVGDEQF |
| CDR3-16 | | |
| other | 62 | ASSDWLAGAKDEQY |
| CDR3-17 | | |
| other | 63 | ASSLVASNEQF |
| CDR3-18 | | |
| other | 64 | ASSLGLLLYNEQF |
| CDR3-19 | | |
| other | 65 | ASSLGDSYEQYF |
| CDR3-20 | | |
| other | 66 | ASSEWGSTGELF |
| CDR3-21 | | |
| other | 67 | ASSEWGSTGELF |
| CDR3-22 | | |
| other | 68 | ASSEYTMGTQY |
| CDR3-23 | | |

1.-201. (canceled)

202. A recombinant nucleic acid encoding a T cell receptor (TCR) comprising a TCR beta chain construct comprising a complementarity determining region 3 (CDR3) having an amino acid sequence set forth in SEQ ID NO: 6.

203. The recombinant nucleic acid of claim 202, wherein the TCR beta chain construct further comprises a complementarity determining region 1 (CDR1), wherein the CDR1 has an amino acid sequence set forth in SEQ ID NO: 4.

204. The recombinant nucleic acid of claim 202, wherein the TCR beta chain construct further comprises a complementarity determining region 2 (CDR2), wherein the CDR2 has an amino acid sequence set forth in SEQ ID NO: 5.

205. The recombinant nucleic acid claim 202, wherein the TCR beta chain construct comprises a variable region having at least 80%, at least 90%, at least 95%, at least 96%, at least 97%, at least 98%, at least 99%, or 100% sequence identity to an amino acid sequence set forth in SEQ ID NO: 12.

206. The recombinant nucleic acid of claim 202, further comprising a TCR alpha chain construct having a CDR1, a CDR2, and a CDR3,
wherein, the CDR1 has an amino acid sequence set forth in SEQ ID NO: 1;
the CDR2 has an amino acid sequence set forth in SEQ ID NO: 2; and,
the CDR3 has an amino acid sequence set forth in SEQ ID NO: 3.

207. The recombinant nucleic acid of claim 202, comprising:
(a) a sequence having at least 80% sequence identity with SEQ ID NOs: 10 or 11; and
(b) a sequence having at least 80% sequence identity with SEQ ID NOs: 7 or 8.

208. The recombinant nucleic acid of claim 206, wherein the TCR alpha chain construct comprises a variable region having at least 80%, at least 90%, at least 95%, at least 96%, at least 97%, at least 98% at least 99%, or 100% sequence identity to an amino acid sequence set forth in SEQ ID NO: 9.

209. The recombinant nucleic acid of claim 202, wherein the TCR comprises:
(a) a beta chain having an amino acid sequence set forth in SEQ ID NOs: 14 or 70, or an amino acid sequence that is at least 80% identical to SEQ ID NOs: 14 or 70,
(b) an alpha chain having an amino acid sequence set forth in SEQ ID NOs: 13 or 69, or an amino acid sequence that is at least 80% identical to SEQ ID NOs: 13 or 69.

210. The recombinant nucleic acid of claim 202, wherein the TCR binds to an epitope from human RAS comprising a mutation G12V.

211. The recombinant nucleic acid of claim 210, wherein the epitope from human RAS comprising the mutation G12V is SEQ ID NOs: 43 or 44.

212. The recombinant nucleic acid of claim 210, wherein the TCR binds to a complex comprising the epitope from human RAS comprising the mutation G12V.

213. The recombinant nucleic acid of claim 202, wherein the TCR binds to a complex comprising an MHC protein encoded by an HLA-A11:01 allele.

214. A recombinant nucleic acid encoding a T cell receptor (TCR) comprising a TCR beta chain construct and a TCR alpha chain construct, wherein the TCR specifically binds to a mutated epitope from human RAS in complex with a human MHC encoded by an HLA-A68:01 allele, wherein the mutated epitope from human RAS is characterized by a G12V mutation.

215. The recombinant nucleic acid of claim 214, wherein the TCR beta chain construct comprises a complementarity determining region 3 (CDR3), wherein the CDR3 has an amino acid sequence of SEQ ID NO: 82.

216. The recombinant nucleic acid of claim 214, wherein the TCR beta chain construct comprises a complementarity determining region 1 (CDR1), wherein the CDR1 has an amino acid sequence of SEQ ID NO: 80.

217. The recombinant nucleic acid of claim 214, wherein the TCR beta chain construct comprises a complementarity determining region 2 (CDR2), wherein the CDR2 has an amino acid sequence of SEQ ID NO: 81.

218. The recombinant nucleic acid of claim 214, wherein the TCR beta chain construct comprises a variable region having at least 80%, at least 90%, at least 95%, at least 96%, at least 98%, at least 99%, or 100% sequence identity to an amino acid sequence set forth in SEQ ID NO: 84.

219. The recombinant nucleic acid of claim 214, further comprising a TCR alpha chain construct having a CDR1, a CDR2, and a CDR3,
wherein, the CDR1 has an amino acid sequence set forth in SEQ ID NO: 77;
the CDR2 has an amino acid sequence set forth in SEQ ID NO: 78; and,
the CDR3 has an amino acid sequence set forth in SEQ ID NO: 79.

220. The recombinant nucleic acid of claim 219, wherein the TCR alpha chain construct comprises a variable region having at least 80%, at least 90%, at least 95%, at least 96%, at least 97%, at least 98%, at least 99% or at least 100% sequence identity to an amino acid sequence set forth in SEQ ID NO: 83.

221. The recombinant nucleic acid of claim 214, wherein the mutated epitope from human RAS comprising the mutation G12V is SEQ ID NOs: 43 or 44.
